(19) United States
(12) Patent Application Publication
(10) Pub. No.: US 2022/0089598 A1
(43) Pub. Date: Mar. 24, 2022

(54) INHIBITORS OF HUMAN IMMUNODEFICIENCY VIRUS REPLICATION

(71) Applicant: VIIV HEALTHCARE UK (No. 5) LIMITED, Brentford, Middlesex (GB)

(72) Inventors: Kyle E. PARCELLA, Branford, CT (US); Christiana IWUAGWU, Branford, CT (US); Kevin M. PEESE, Branford, CT (US)

(21) Appl. No.: 17/272,377

(22) PCT Filed: Sep. 12, 2019
(86) PCT No.: PCT/IB2019/057710

§ 371 Date: Mar. 1, 2021

Related U.S. Application Data
(60) Provisional application No. 62/733,259, filed on Sep. 19, 2018.
(60) Provisional application No. 62/731,196, filed on Sep. 14, 2018.

(51) Int. Cl. C07D 487/04

(52) U.S. Cl. CPC C07D 487/04

(57) ABSTRACT

Compounds of Formula I, including pharmaceutically acceptable salts thereof, and compositions and methods for treating human immunodeficiency virus (HIV) infection are set forth:

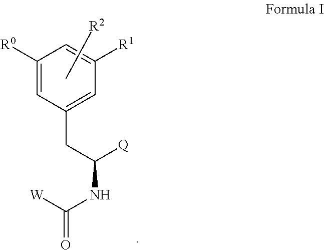

### FIELD OF THE INVENTION

The invention relates to compounds, compositions, and methods for the treatment of human immunodeficiency virus (HIV) infection. More particularly, the invention provides novel inhibitors of HIV, pharmaceutical compositions containing such compounds, and methods for using these compounds in the treatment of HIV infection. The invention also relates to methods for making the compounds hereinafter described.

### BACKGROUND OF THE INVENTION

Acquired immunodeficiency syndrome (AIDS) is the result of infection by HIV.

HIV continues to be a major global public health issue. In 2015, an estimated 36.7 million people were living with HIV (including 1.8 million children)—a global HIV prevalence of 0.8%. The vast majority of this number live in low- and middle-income countries. In the same year, 1.1 million people died of AIDS-related illnesses.

Current therapy for HIV-infected individuals consists of a combination of approved anti-retroviral agents. Close to four dozen drugs are currently approved for HIV infection, either as single agents, fixed dose combinations or single tablet regimens; the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (PIs), integrase strand transfer inhibitors (INSTIs), or entry inhibitors (one, maraviroc, targets the host CCR5 protein, while the other, enfuvirtide, is a peptide that targets the gp41 region of the viral gp160 protein). In addition, a pharmacokinetic enhancer (cobicistat or ritonavir) can be used in combinations with antiretroviral agents (ARVs) that require boosting.

Despite the armamentarium of agents and drug combinations, there remains a medical need for new anti-retroviral agents. High viral heterogeneity, drug-associated toxicity, tolerability problems, and poor adherence can all lead to treatment failure and may result in the selection of viruses with mutations that confer resistance to one or more antiretroviral agents or even multiple drugs from an entire class (Beyrer, C., Pozniak A. HIV drug resistance—an emerging threat to epidemic control. N. Engl. J. Med. 2017, 377, 1605-1607; Gupta, R. K., Gregson J., et al. HIV-1 drug resistance before initiation or re-initiation of first-line antiretroviral therapy in low-income and middle-income countries: a systematic review and meta-regression analysis. Lancet Infect. Dis. 2017, 18, 346-355; Zazzi, M., Hu, H., Prosperi, M. The global burden of HIV-1 drug resistance in the past 20 years. PeerJ. 2018, DOI 10.7717/peerj.4848). As a result, new drugs are needed that are easier to take, have high genetic barriers to the development of resistance and have improved safety over current agents. In this panoply of choices, novel mechanisms of action (MOAs) that can be used as part of the preferred antiretroviral therapy (ART) can still have a major role to play since they should be effective against viruses resistant to current agents.

Certain potentially therapeutic compounds have now been described in the art and set forth in Blair, Wade S. et. al. Antimicrobial Agents and Chemotherapy (2009), 53(12), 5080-5087, Blair, Wade S. et al. PLoS Pathogens (2010), 6(12), e1001220, Thenin-Houssier, Suzie; Valente, Susana T. Current HIV Research, 2016, 14, 270-282, and PCT Patent applications with the following numbers: WO 2012065062, WO 2013006738, WO 2013006792, WO 2014110296, WO 2014110297, WO 2014110298, WO 2014134566, WO 2015130964, WO2015130966, WO 2016033243, WO2018035359, WO2018203235, WO 2019161017, and WO 2019161280.

What is now needed in the art are additional compounds which are novel and useful in the treatment of HIV. Additionally, these compounds should provide advantages for pharmaceutical uses, for example, with regard to one or more of their mechanisms of action, binding, inhibition efficacy, target selectivity, solubility, safety profiles, bioavailability or reduced frequency of dosing. Also needed are new formulations and methods of treatment which utilize these compounds.

### SUMMARY OF THE INVENTION

In one aspect, the present invention discloses compound of Formula I, or a pharmaceutically acceptable salt thereof:

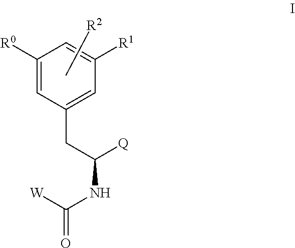

wherein:
R⁰, R¹, and R² are each independently selected from hydrogen, Cl, F, —OMe, —CN, or —CH₃ with the proviso that substituents Cl, —OMe, and —CH₃ may not be used more than twice and substituent —CN may not be used more than once;
Q is selected from:

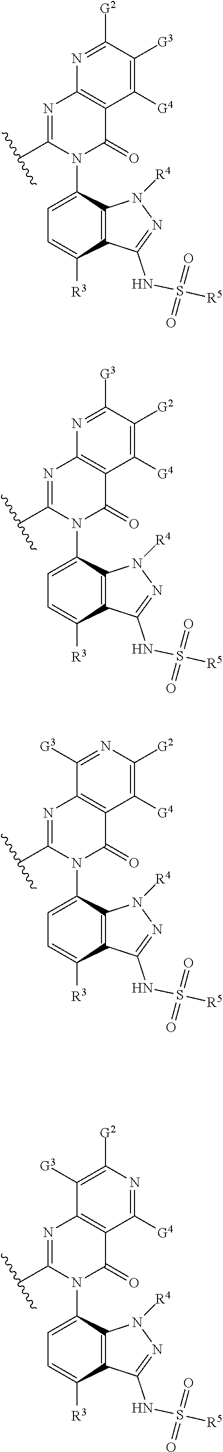

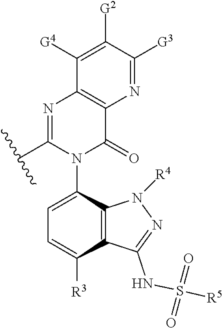

G² is one of the following:

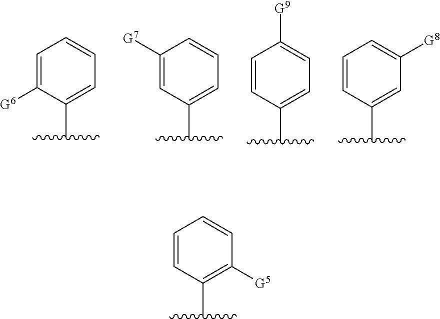

wherein the benzene ring may be further substituted up to two times with fluorine and up to two times with chlorine with the proviso that no more than three substituents directly connected to the benzene ring are a halogen and no more than two substituents directly connected to the benzene ring are a chloride;
G³ and G⁴ are independently selected from hydrogen, methyl, fluoro, chloro, or OC₁-C₂ alkyl with the proviso that at least one of G³ and G⁴ must be hydrogen;
G⁵ is hydrogen, methyl, fluoro, chloro, OC₁-C₃ alkyl, cyano, —CH₂OH, or —SO₂(C₁-C₃ alkyl);
G⁶ is hydrogen, methyl, fluoro, chloro, or OC₁-C₃ alkyl;
G⁷ is hydrogen, methyl, fluoro, chloro, OC₁-C₃ alkyl, or COOH;
G⁸ is hydrogen, C₁-C₄ alkyl, fluoro, chloro, OC₁-C₃ alkyl, COOH, —CN, —CH₂OH, or —SO₂(C₁-C₃ alkyl)
G⁹ is hydrogen, C₁-C₄ alkyl, fluoro, chloro, OC₁-C₃ alkyl, COOH, CO-morpholine, C(CH₃)₂CH₂OH, or —SO₂-morpholine wherein C₁-C₄ alkyl is optionally substituted with 1-3 fluorines;
R³ is hydrogen, Cl, or F;
R⁴ is hydrogen, C₁-C₃ alkyl, or cyclopropyl wherein C₁-C₃ alkyl is optionally substituted with 1-3 fluorines and cyclopropyl is optionally substituted with 1-2 fluorines.
R⁵ is C₁-C₃ alkyl or C₃-C₄ cycloalkyl;
W is selected from:

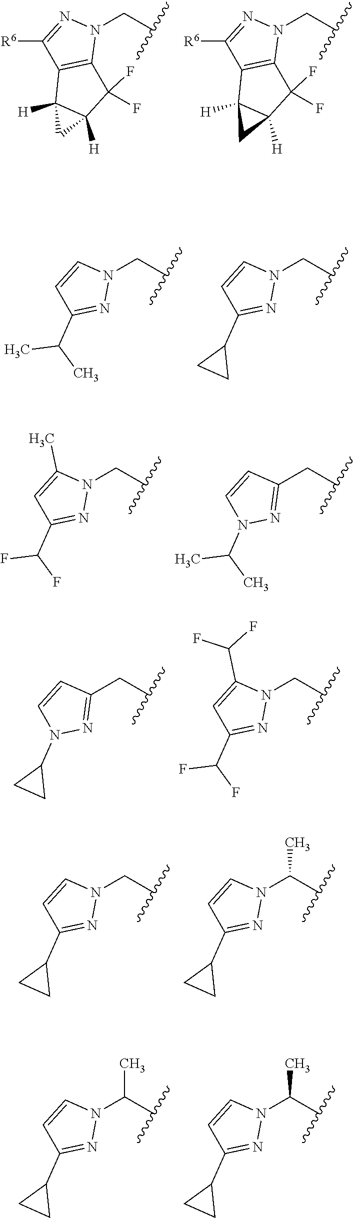

wherein R⁶ and R⁷ are independently selected from methyl optionally substituted with 1 to 3 fluorines.

In another aspect, the present invention discloses a pharmaceutical composition comprising a compound of Formula I or a pharmaceutically acceptable salt thereof.

In another aspect, the present invention discloses a method of treating HIV infection comprising administering a composition comprising a compound of Formula I or a pharmaceutically acceptable salt thereof to a patient.

In another aspect, the present invention discloses a compound of Formula I or pharmaceutically acceptable salt thereof for use in therapy.

In another aspect, the present invention discloses a compound of Formula I or pharmaceutically acceptable salt thereof for use in treating HIV infection.

In another aspect, the present invention discloses the use of a compound of Formula I or pharmaceutically acceptable salt thereof in the manufacture of a medicament for the treatment of HIV infection.

### DETAILED DESCRIPTION OF THE EMBODIMENTS

In one embodiment, the present invention discloses a compound or salt wherein G² contains a fluorine. In another embodiment, the present invention discloses a compound or salt of Formula I wherein the substituents of G² are restricted to hydrogen and fluorine.

In another embodiment, the present invention discloses a compound or salt wherein G² is one of the following:

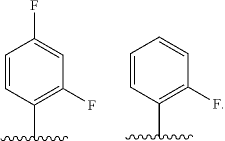

In another embodiment, the present invention discloses a compound or salt wherein G² is one of the following:

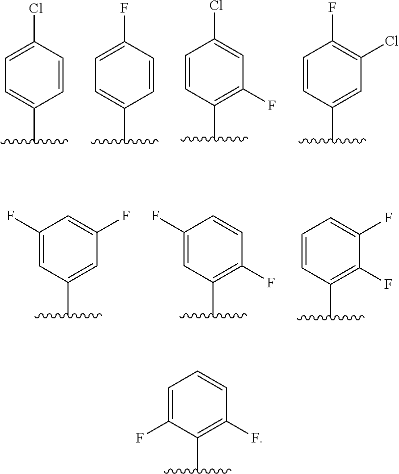

In another embodiment, the present invention discloses a compound or salt wherein G² is one of the following:

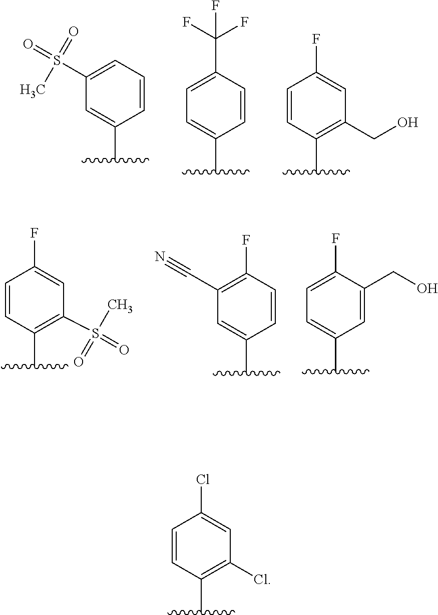

In another embodiment, the present invention discloses a compound or salt wherein G² is one of the following:

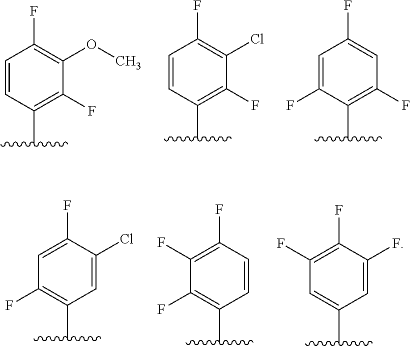

In one embodiment, the present invention discloses a compound or salt wherein W is

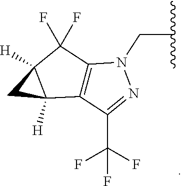 [SMILES: *Cn1nc(C(F)(F)F)c2c1C(F)(F)[C@@H]1C[C@H]21]

In another embodiment, the present invention discloses a compound or salt wherein W is

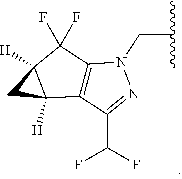 [SMILES: *Cn1nc(C(F)F)c2c1C(F)(F)[C@@H]1C[C@H]21]

In another embodiment, the present invention discloses a compound or salt wherein W is one of the following:

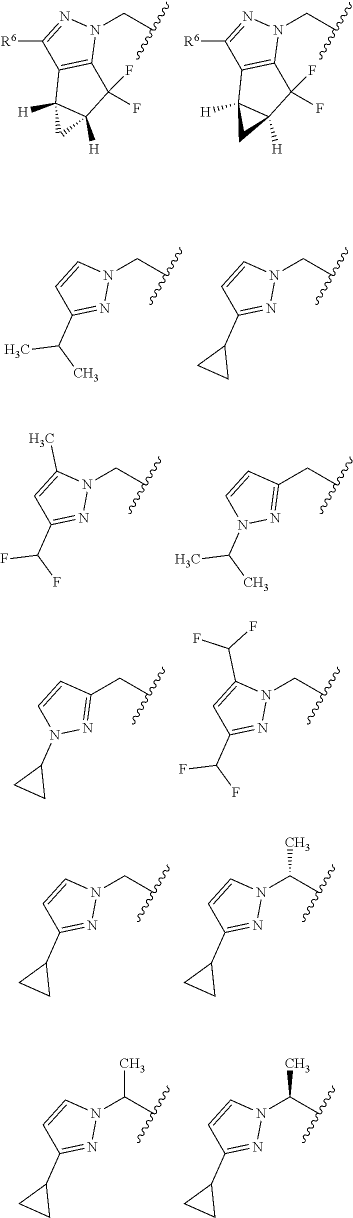

wherein R⁶ is methyl optionally substituted with one fluorine and R⁷ is methyl optionally substituted with 1 to 3 fluorines.

In one embodiment, the present invention discloses a compound or salt wherein R⁰ is F, R¹ is F, and R² is H. In another embodiment, the present invention discloses a compound or salt wherein R⁰, R¹, and R² are each independently selected from hydrogen, F, Cl or —CH₃ with the proviso that at least one of the group R⁰, R¹ and R² is hydrogen and that R² is not hydrogen if R⁰ and R¹ are both F. In another embodiment, the present invention discloses a compound or salt wherein R⁰, R¹, and R² are each independently selected from Cl, F, —OMe, —CN, or —CH₃ with the proviso that substituents Cl, —OMe, and —CH₃ may not be used more than twice and substituent —CN may not be used more than once.

In one embodiment, the present invention discloses a compound or salt wherein R³ is chloride; R⁴ is methyl, 2,2-difluoroethyl, or 2,2,2-trifluoroethyl; and R⁵ is methyl or cyclopropyl.

In one embodiment, the present invention discloses a compound or salt wherein Q is

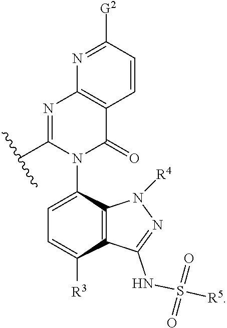

In another embodiment, the present invention discloses a compound or salt wherein Q is

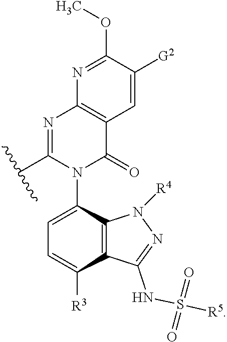

In another embodiment, the present invention discloses a compound or salt wherein Q is

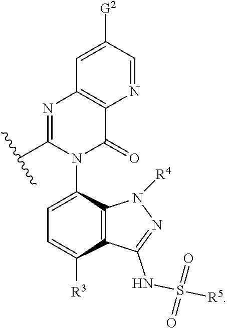

Preferred embodiments of the invention include compounds, and salts thereof, comprising any combination of the embodiments set forth above.

In one embodiment, the present invention discloses a compound or salt, selected from the group consisting of:

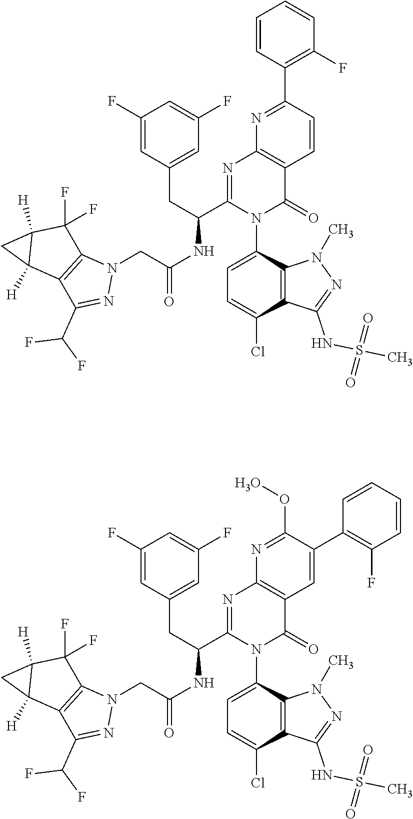

and pharmaceutically acceptable salts thereof.

In another embodiment, the present invention discloses a compound or salt, selected from the group consisting of:

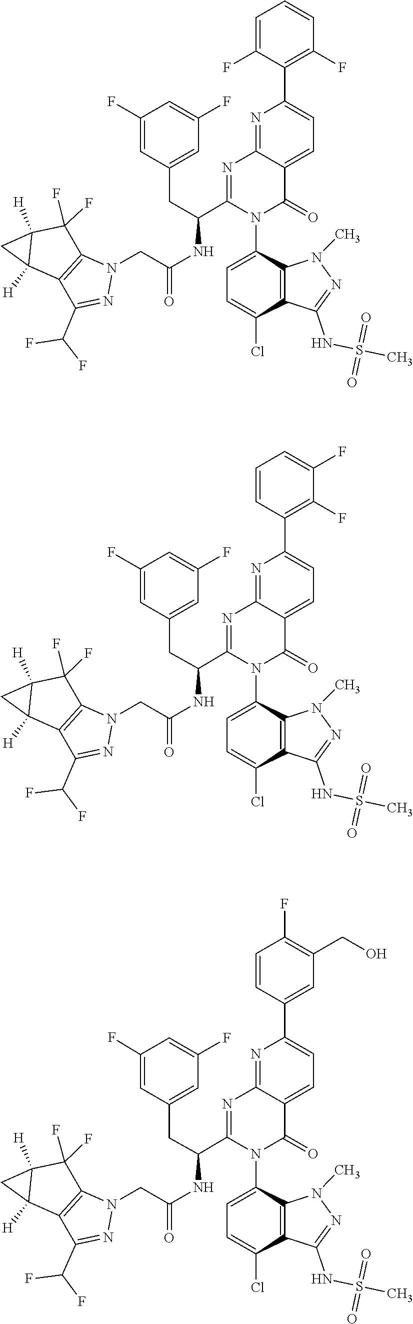

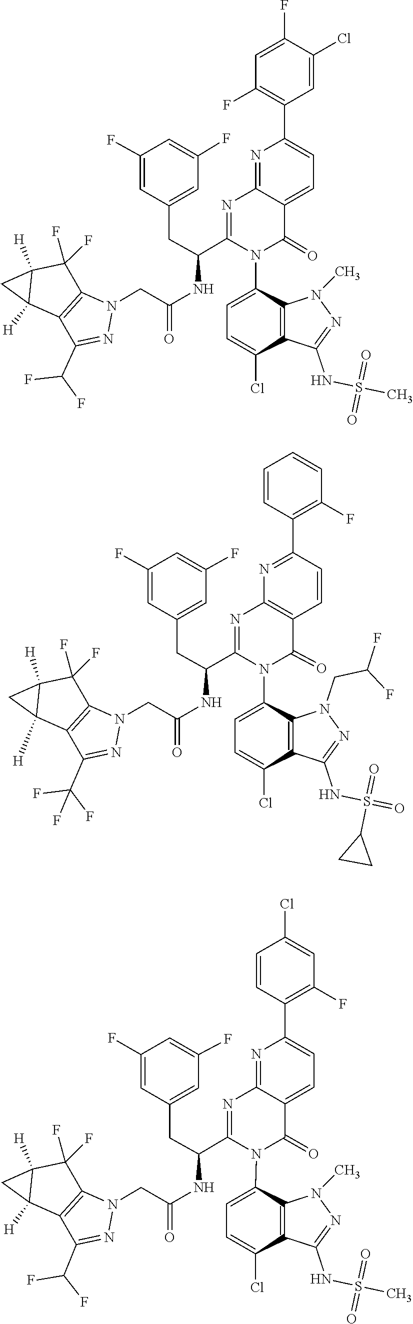

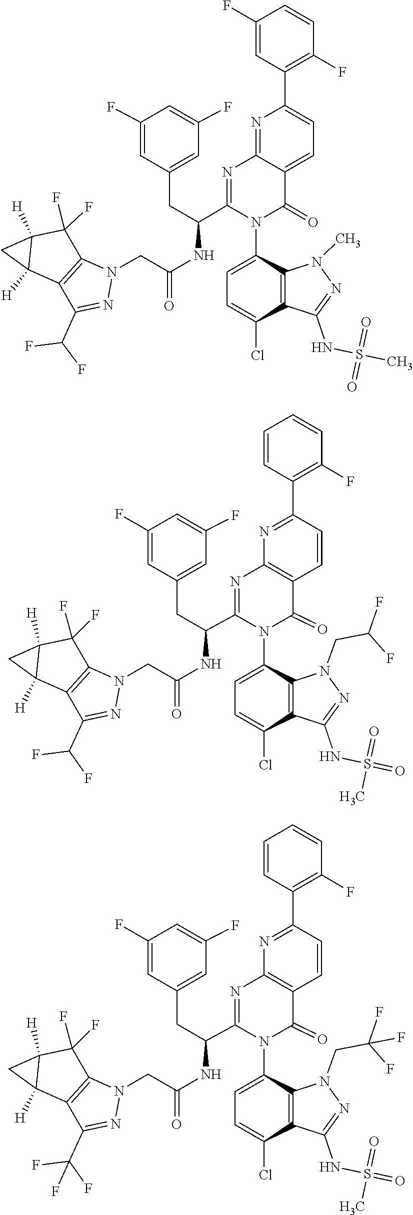

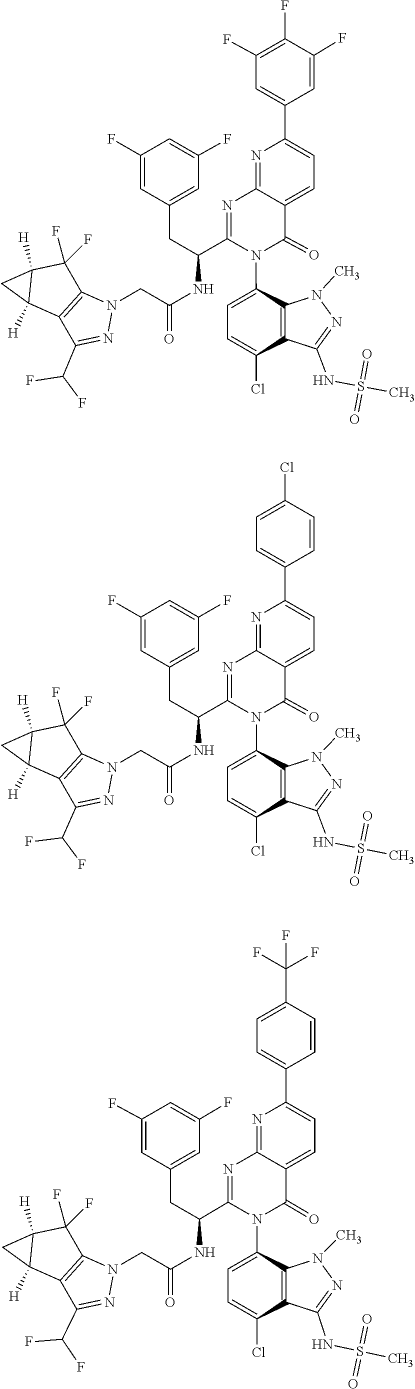

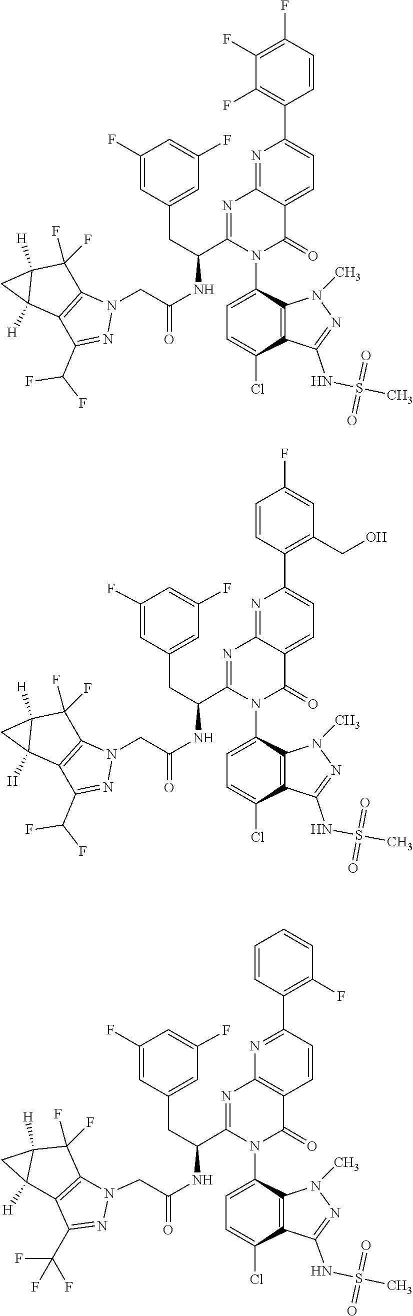

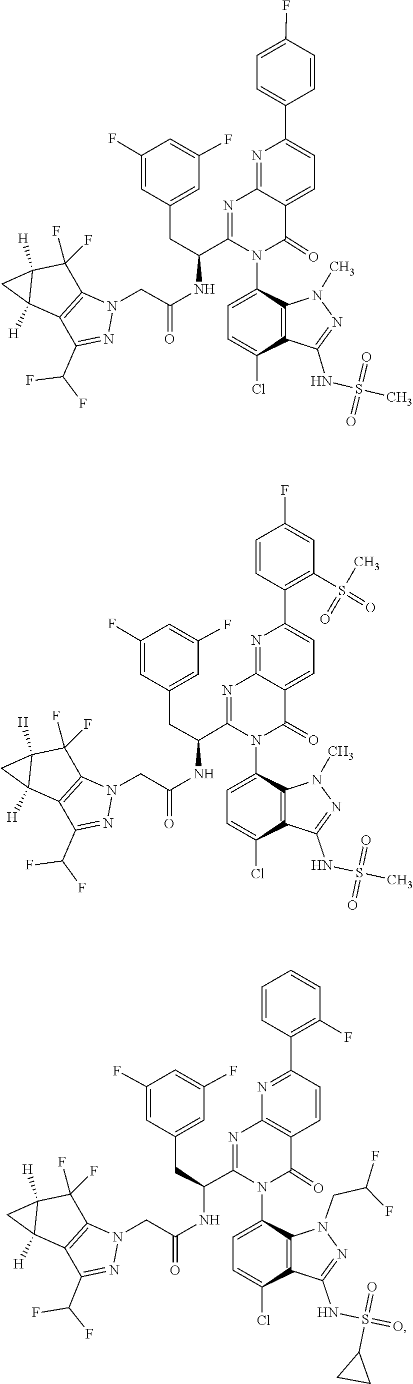

and acceptable salts thereof pharmaceutically

In another embodiment, the present invention discloses a compound or salt, selected from the group consisting of:

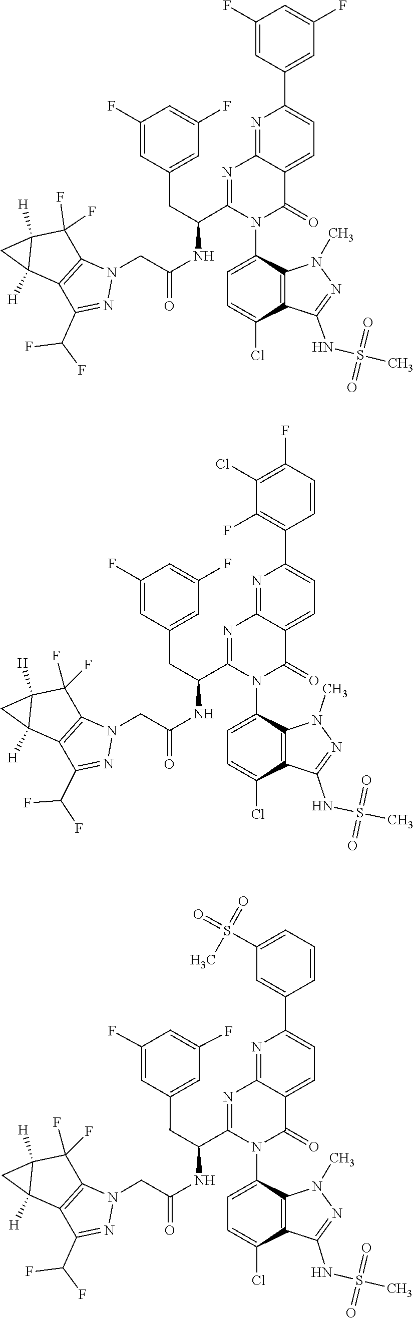

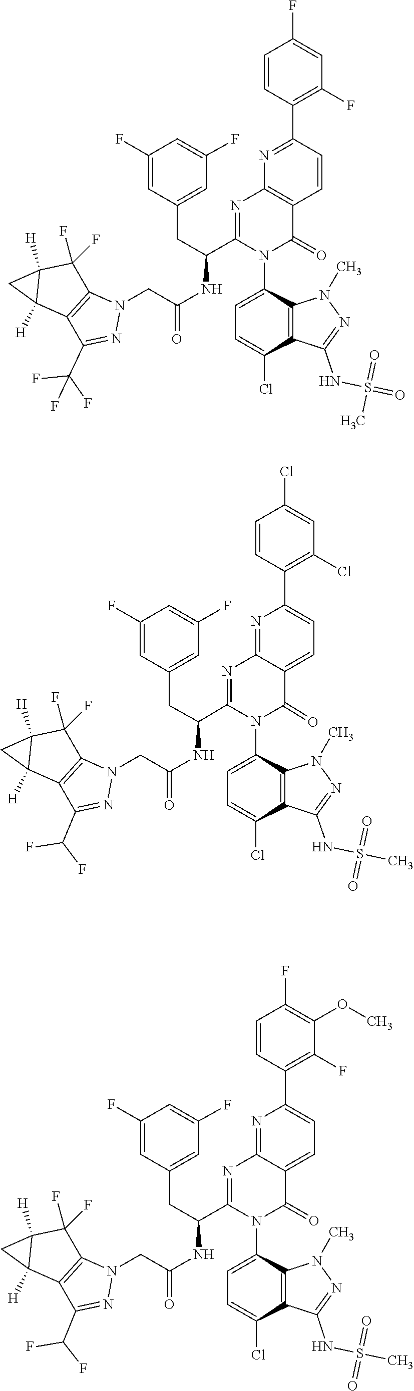

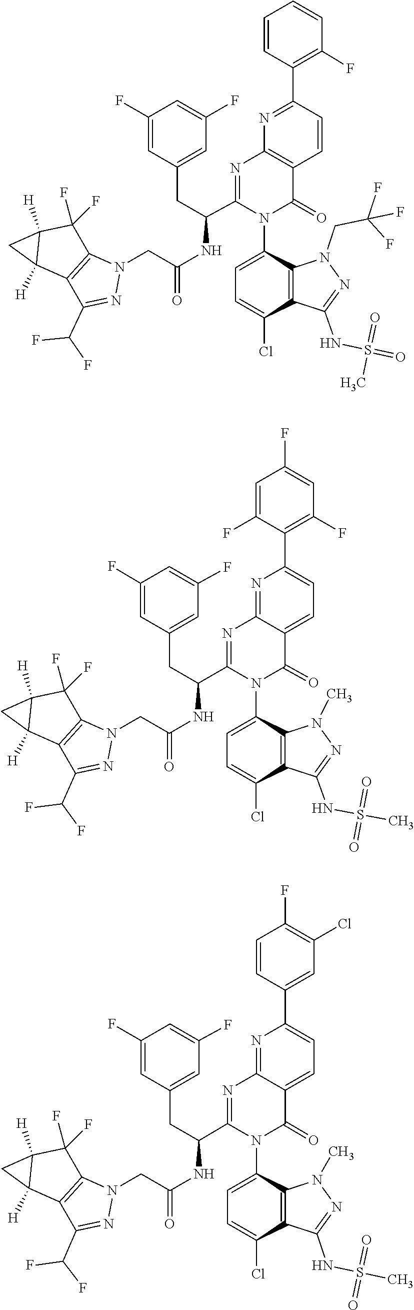

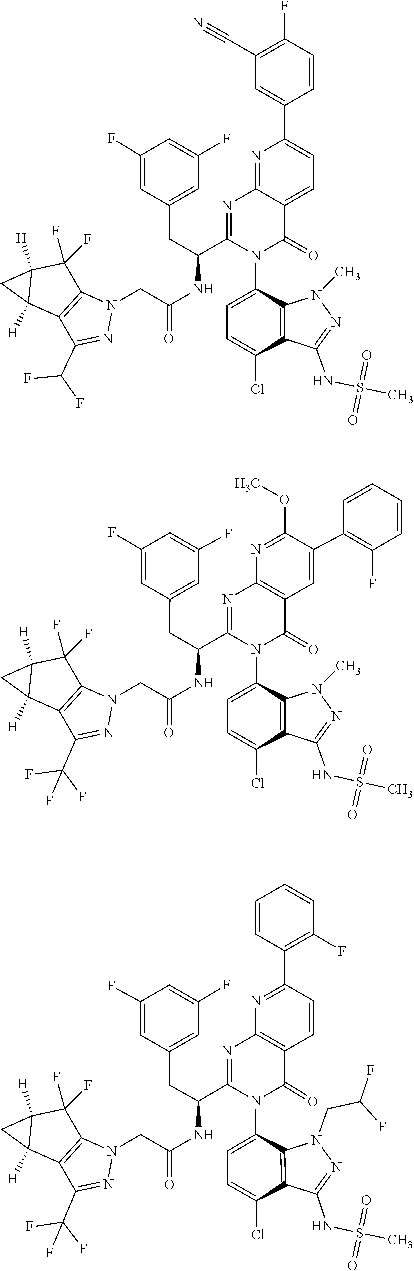

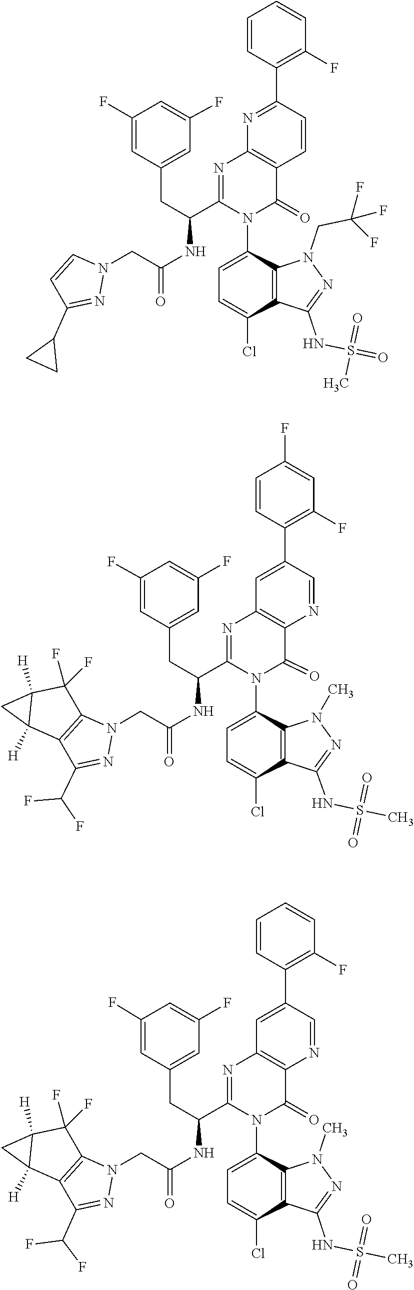

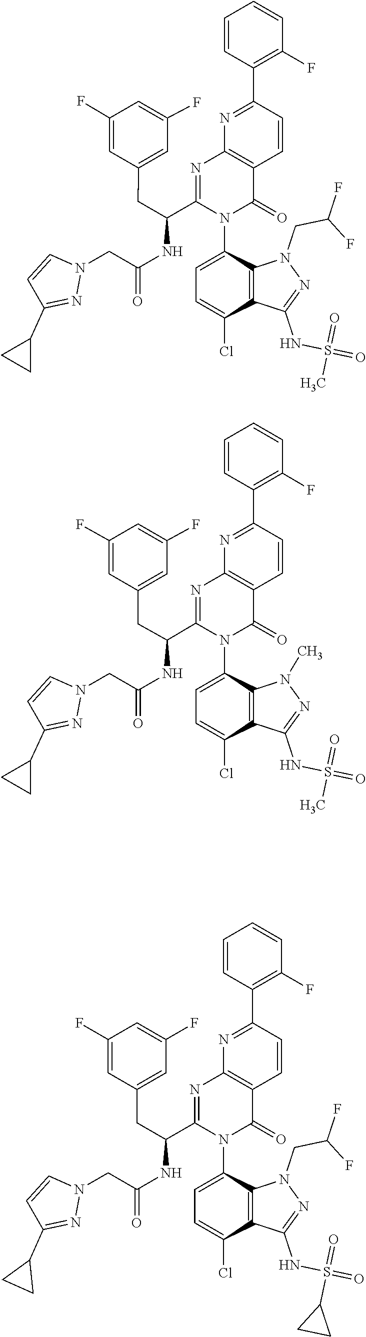

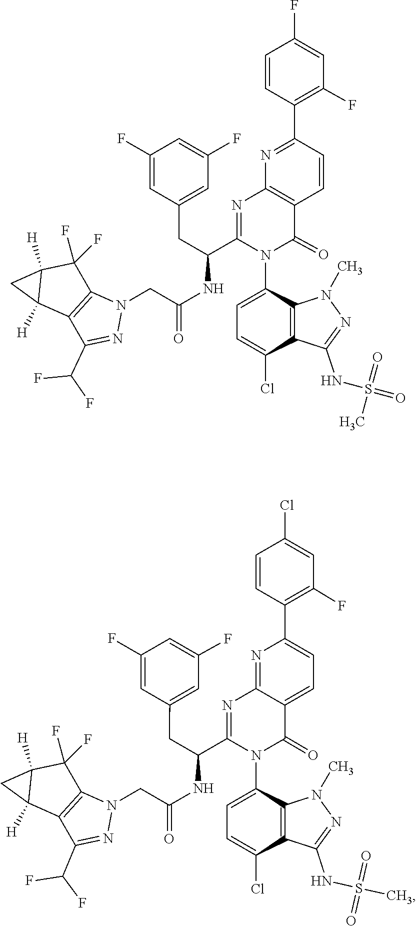

and pharmaceutically acceptable salts thereof.

The salts of compounds of formula (I) are pharmaceutically acceptable. Such salts may be acid addition salts or base addition salts. For a review of suitable pharmaceutically acceptable salts see Berge et al, J. Pharm, Sci., 66, 1-19, 1977. In an embodiment, acid addition salts are selected from the hydrochloride, hydrobromide, hydroiodide, sulphate, bisulfate, nitrate, phosphate, hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate. In an embodiment, base addition salts include metal salts (such as sodium, potassium, aluminium, calcium, magnesium and zinc) and ammonium salts (such as isopropylamine, diethylamine, diethanolamine salts). Other salts (such as trifluoroacetates and oxalates) may be used in the manufacture of compounds of formula (I) and their pharmaceutically acceptable salts, and are included within the scope of the invention. All possible stoichiometric and non-stoichiometric forms of the salts of compounds of formula (I) are included within the scope of the invention. Acid and base addition salts may be prepared by the skilled chemist, by treating a compound of formula (I) with the appropriate acid or base in a suitable solvent, followed by crystallisation and filtration.

Some of the compounds of the invention exist in stereoisomeric forms. The invention includes all stereoisomeric forms of the compounds including enantiomers and diastereromers including atropisomers. The term homochiral is used as a descriptor, per accepted convention, to describe a structure which is a single stereoisomer. Absolute stereochemistry was not assigned in all cases. Thus, the compound is drawn at the chiral center as unspecified but labelled as homochiral and in the procedures it is identified by its properties such as for example first eluting off a normal or chiral column per the conventions of chemists. It should be noted that the provided experimental procedures teach how to make the exact compound even if not drawn with absolute configuration. Methods of making and separating stereoisomers are known in the art. The invention includes all tautomeric forms of the compounds. The invention includes atropisomers and rotational isomers.

For the compounds of Formula I, the scope of any instance of a variable substituent can be used independently with the scope of any other instance of a variable substituent. As such, the invention includes combinations of the different aspects. In some examples, the stereochemistry of all the centers were not unambiguously assigned so they can be referred to as diastereomer 1 and diastereomer 2 or enantiomer 1 or enantiomer 2 etc. and these are understood by chemists skilled in the art. In other cases, atropisomers can be observed and these are understood to convert at slow or fast rates or even not at all depending on the conditions for handling the compound. These are referred to as mixtures of atropisomers where they interconvert at ambient temperatures or as atropisomer 1 and atropisomer 2 where they were isolated. Since the compounds are identified by their properties rather than exact structural assignment from a crystal structure, it is understood in the art that where not specified, atropisomers are covered and inferred to be covered by the chemical structure.

In the method of this invention, preferred routes of administration are oral, by injection to deliver intramuscularly, and by injection to deliver subcutaneously. Therefore, preferred pharmaceutical compositions are those compositions suitable for these routes of administration, for example tablets or injectable compositions.

The compounds of this invention are believed to have as their biological target the HIV Capsid and thus their mechanism of action is to modify in one or more ways the function of the HIV capsid.

The compounds of the present invention and their salts, solvates, or other pharmaceutically acceptable derivatives thereof, may be employed alone or in combination with other therapeutic agents. The compounds of the present invention and any other pharmaceutically active agent(s) may be administered together or separately and, when administered separately, administration may occur simultaneously or sequentially, in any order. The amounts of the compounds of the present invention and the other pharmaceutically active agent(s) and the relative timings of administration will be selected in order to achieve the desired combined therapeutic effect. The administration in combination of a compound of the present invention and salts, solvates, or other pharmaceutically acceptable derivatives thereof with other treatment agents may be in combination by administration concomitantly in: (1) a unitary pharmaceutical composition including multiple compounds; or (2) separate pharmaceutical compositions each including one of the compounds. Alternatively, the combination may be administered separately in a sequential manner wherein one treatment agent is administered first and the other second or vice versa, and the different agents could be administered on different schedules if appropriate. Such sequential administration may be close in time or remote in time.

As such, the compounds of the present invention may be used in combination with one or more agents useful in the prevention or treatment of HIV.

### EXAMPLES

The compounds of the invention according to the various embodiments can be made by various methods available in the art, including those of the following schemes in the specific examples which follow. The structure numbering and variable numbering shown in the synthetic schemes may be distinct from, and should not be confused with, the structure or variable numbering in the claims or the rest of the specification. The variables in the schemes are meant only to illustrate how to make some of the compounds of the invention.

Abbreviations used in the schemes generally follow conventions used in the art. Some specific chemical abbreviations used in the examples are defined as follows: “DMF” for N,N-dimethylformamide; “MeOH” for methanol; “Ar” for aryl; “TFA” for trifluoroacetic acid; “BOC” for t-butoxycarbonate, “DMSO” for dimethylsulfoxide; “h” for hours; “rt” for room temperature or retention time (context will dictate); “min” for minutes; “EtOAc” for ethyl acetate; “THF” for tetrahydrofuran; “Et₂O” for diethyl ether; “DMAP” for 4-dimethylaminopyridine; “DCE” for 1,2-dichloroethane; “ACN” for acetonitrile; “DME” for 1,2-dimethoxyethane; “HATU” for (1-[Bis(dimethylamino)methylene]-1H-1,2,3-triazolo[4,5-b]pyridinium 3-oxid hexafluorophosphate); “DIEA” or “DIPEA” for diisopropylethylamine.

Certain other abbreviations as used herein, are defined as follows: “1×” for once, “2×” for twice, “3×” for thrice, “° C.” for degrees Celsius, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “min” for minute or minutes, “h” for hour or hours, “rt” for room temperature, “RT” for retention time, “atm” for atmosphere, “psi” for pounds per square inch, “conc.” for concentrate, “sat” or “sat'd” for saturated, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “1H” for proton, “δ” for delta, “s” for singlet, “d” for doublet, “t” for triplet, “q” for quartet, “in” for multiplet, “br” for broad, “Hz” for hertz, and “α”, “β”, “R”, “S”, “E”, and “Z” are stereochemical designations familiar to one skilled in the art.

The following examples are provided by way of illustration only and should not be construed as limiting the scope of the invention.

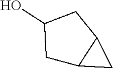

### Bicyclo[3.1.0]hexan-3-ol

To a stirred solution of cyclopent-3-enol (130 g, 1545 mmol) in DCM (1200 mL), was added Diethyl zinc (1.0 M in Hexane, 3091 mL, 3091 mmol) drop wise at 0-5° C. over a period of 3 h followed by drop wise addition of Diiodomethane (249 mL, 3091 mmol) in DCM (300 mL) over a period of 1 h at 0° C. Reaction mixture was allowed to warm to 27° C. (Note: white precipitation was observed) and stirred for 16 h under N2 atmosphere. Progress of the reaction was monitored by TLC (SiO₂, 20% EtOAc/pet, Rf=0.3, UV-inactive, PMA-active). After completion, the reaction mixture was quenched with aq saturated NH₄Cl solution (1.5 L) and filtered through celite bed. The aqueous layer was extracted with DCM (2×1000 mL). The combined organic layers were dried over anhydrous Na₂SO₄, filtered and the filtrate was concentrated under reduced pressure to afford crude bicyclo[3.1.0]hexan-3-ol (180 g, Yield: Crude) as reddish liquid. ¹H NMR (400 MHz, CHLOROFORM-d) δ=4.41-4.35 (m, 1H), 2.18-2.05 (m, 2H), 1.73 (d, J=13.9 Hz, 2H), 1.35-1.25 (m, 2H), 1.21-1.14 (m, 1H), 0.57-0.43 (m, 2H). GCMS: m/z=98.1).

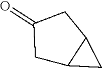

### Bicyclo[3.1.0]hexan-3-one

To a stirred solution of bicyclo[3.1.0]hexan-3-ol (210 g, 2054 mmol) in DCM (5000 mL), was added Dess-martin periodinane (954 g, 2249 mmol) portion wise at 0° C. and allowed to warm to 27° C. under N2 atmosphere and stirred for 16 h. Progress of the reaction was monitored by TLC (SiO₂, 20% Acetone/Hex, Rf=0.3, UV in-active, PMA-active). After completion, the reaction mixture was filtered through celite bed and the filtrate was washed with 1N NaOH solution (8×1000 mL) and extracted with DCM (5×1000 mL). The combined organic layers were dried over anhydrous Na₂SO₄ and filtered. The filtrate was concentrated under reduced pressure (Bath temperature: 20° C.) to get crude compound as brown liquid which was purified by downward distillation at 70° C. to afford bicyclo[3.1.0]hexan-3-one (125 g, Yield: 62%, Pale yellow viscous liquid. ¹H NMR (400 MHz, CHLOROFORM-d) δ=2.61-2.54 (m, 2H), 2.17-2.12 (m, 2H), 1.54-1.46 (m, 2H), 0.92-0.86 (m, 1H), −0.01-−0.08 (m, 1H), GCMS: M/Z=96.1).

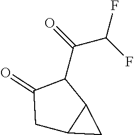 [SMILES: O=C1CC2CC2C1C(=O)C(F)F]

### 2-(2,2-difluoroacetyl)bicyclo[3.1.0]hexan-3-one

To a stirred solution of bicyclo[3.1.0]hexan-3-one (125 g, 1274 mmol) in THF (1500 mL), was added LDA (2.0 M in THF, 0.701 L, 1402 mmol) at −78° C. under N2 atmosphere and stirred for 1 h followed by the addition Ethyldifluoroacetate (174 g, 1402 mmol) in THF (300 mL) slowly at −78° C. for 30 min. After the addition the reaction mixture was allowed to warm to 27° C. and stirred for 1 h under N2 atmosphere. Progress of the reaction was monitored by TLC (SiO₂, 20% Acetone/Hexane, Rf=0.3, UV-active). After completion, the reaction mixture was quenched with 1N HCl (2000 mL) and stirred for 30 min and extracted with EtOAc (3×1000 mL). The combined organic layers were washed with brine (1000 mL), dried over anhydrous Na₂SO₄ and filtered. The filtrate was concentrated under reduced pressure to afford 2-(2,2-difluoroacetyl)bicyclo[3.1.0]hexan-3-one (180 g, Yield: 71.2%, pale yellow viscous liquid. ¹H NMR (400 MHz, CHLOROFORM-d) δ=6.18 (t, J=54.8 Hz, 1H), 2.70-2.62 (m, 1H), 2.35 (d, J=19.4 Hz, 1H), 2.14 (br s, 1H), 1.26-1.21 (m, 1H), 1.04-1.03 (m, 1H), 0.22-0.21 (m, 1H), LCMS: M/Z=173.17).

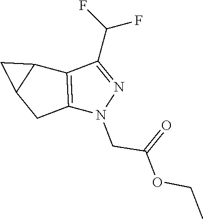 [SMILES: CCOC(=O)Cn1nc(C(F)F)c2c1CC1CC21]

### Ethyl 2-(3-(difluoromethyl)-3b,4,4a,5-tetrahydro-1H-cyclopropa[3,4]cyclopenta[1,2-c]pyrazol-1-yl)acetate

To a stirred solution of 2-(2,2-difluoroacetyl)bicyclo[3.1.0]hexan-3-one (180 g, 910 mmol) in Ethanol (2 L), was added ethyl 2-hydrazinylacetate hydrochloride (422 g, 2729 mmol) followed by the addition of sulfuric acid (20 mL, 375 mmol) at 27° C. under N2 atmosphere and stirred for 30 min. The reaction mixture was further heated to 100° C. and stirred for 16 h. Progress of the reaction was monitored by TLC (SiO₂, 20% Acetone/Hexane, Rf=0.3, UV-active). After completion, the reaction mixture was evaporated under reduced pressure, the residue was dissolved in EtOAc (2000 mL) and washed with water (2×1 L), brine (1.0 L), dried over anhydrous Na₂SO₄ and filtered. The filtrate was concentrated under reduced pressure to obtain crude (N66158-21-A1, 440 g). The above crude compound was purified by column chromatography (silica gel: 100-200 mesh) with 0-2% Acetone/Pet as eluent. The fractions containing product were collected and concentrated under reduced pressure to afford ethyl2-(3-(difluoromethyl)-3b,4,4a,5-tetrahydro-1H-cyclopropa[3,4]cyclopenta[1,2-c]pyrazol-1-yl)acetate (110 g, Yield: 46.4%, as an off white solid. ¹H NMR (400 MHz, DMSO-d₆) δ=6.86 (t, J=54.8 Hz, 1H), 4.93 (s, 2H), 4.14 (q, J=7.2 Hz, 2H), 2.88-2.79 (m, 1H), 2.76-2.68 (m, 1H), 2.14-2.04 (m, 2H), 1.19 (t, J=7.2 Hz, 3H), 1.10-1.03 (m, 1H), 0.14 (q, J=4.3 Hz, 1H). LCMS M+H=257.13.

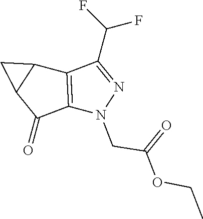 [SMILES: CCOC(=O)Cn1nc(C(F)F)c2c1C(=O)C1CC21]

### Ethyl 2-(3-(difluoromethyl)-5-oxo-3b,4,4a,5-tetrahydro-1H-cyclopropa[3,4]cyclopenta[1,2-c]pyrazol-1-yl)acetate

To a stirred solution of ethyl 2-(3-(difluoromethyl)-3b,4,4a,5-tetrahydro-1H-cyclopropa[3,4]cyclopenta[1,2-c]pyrazol-1-yl)acetate (110 g, 422 mmol) and celite (395 g) in Cyclohexane (3.5 L) at 0° C., pyridinium dichromate (794 g, 2110 mmol) was added portion wise followed by the addition of tert-butyl hydro peroxide (355 mL, 2130 mmol) drop wise over a period of 10 min under N2 atmosphere. The reaction mixture was warmed to 27° C. and stirred for 48 h. Progress of the reaction was monitored by TLC (SiO₂, 30% Acetone/pet, Rf=0.4, UV-active). After completion, the reaction mixture was filtered and washed with EtOAc (1000 mL). The organic layer was washed with saturated aq. Na₂S₂O₃ (2×500 ml), saturated FeSO₄ (300 ml) and brine (500 ml). The organic layer was dried over anhydrous Na₂SO₄, filtered and evaporated under reduced pressure to obtain crude compound (150 g).

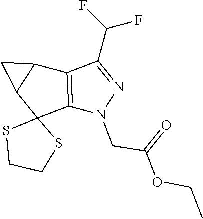 [SMILES: CCOC(=O)Cn1nc(C(F)F)c2c1C1(SCCS1)C1CC21]

### Ethyl 2-(3-(difluoromethyl)-4,4a-dihydrospiro[cyclopropa[3,4]cyclopenta[1,2-c]pyrazole-5,2′-[1,3]dithiolane]-1(3bH)-yl)acetate

To a stirred solution of ethyl 2-(3-(difluoromethyl)-5-oxo-3b,4,4a,5-tetrahydro-1H-cyclopropa[3,4]cyclopenta[1,2-c]pyrazol-1-yl)acetate (75 g, 269 mmol) in DCM (1500 mL), was added ethane-1,2-dithiol (43.0 mL, 511 mmol) and followed by the addition of Boron trifluoride acetic acid (72.6 mL, 511 mmol) at 27° C. under N2 atmosphere and stirred for 16 h at 27° C. Progress of the reaction was monitored by TLC (SiO₂, 20% Acetone/Pet, Rf=0.35, UV-Active). After completion, the reaction mixture was cooled to 0° C. and neutralized with saturated NaHCO₃ (500 mL) and extracted with DCM (2×1000 mL). The combined organics were washed with brine (1000 mL), dried over anhydrous Na₂SO₄, filtered and evaporated under reduced pressure to obtain crude N66187-25-A1 as brown liquid. The above crude was purified by column chromatography using silica gel (100-200 mesh) with 5-10% EtOAc/Pet as eluent. The fractions containing product were collected and concentrated under reduced pressure to afford ethyl 2-(3-(difluoromethyl)-4,4a-dihydrospiro[cyclopropa[3,4]cyclopenta[1,2-c]pyrazole-5,2′-[1,3]dithiolane]-1(3bH)-yl)acetate (80 g, Yield:74.0%) as an Off-white solid. ¹H NMR (400 MHz, CHLOROFORM-d) δ=6.61 (t, J=55.2 Hz, 1H), 5.00-4.85 (m, 2H), 4.29-4.19 (m, 2H), 3.55-3.46 (m, 4H), 2.63-2.53 (m, 1H), 2.49-2.38 (m, 1H), 1.30-1.24 (m, 4H), 0.65-0.60 (m, 1H). LCMS M+H=346.9.

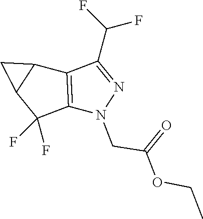 [SMILES: CCOC(=O)Cn1nc(C(F)F)c2c1C(F)(F)C1CC21]

### Ethyl 2-(3-(difluoromethyl)-5,5-difluoro-3b,4,4a,5-tetrahydro-1H-cyclopropa[3,4]cyclopenta[1,2-c]pyrazol-1-yl)acetate

To a stirred solution of 1,3-dibromo-5,5-dimethylimidazolidine-2,4-dione (26.3 g, 92 mmol) in DCM (20 mL) was added HF-pyridine (2.460 g, 24.83 mmol) at −70° C. under N2 atmosphere and stirred for 30 min followed by the addition of ethyl 2-(3-(difluoromethyl)-4,4a-dihydrospiro[cyclopropa[3,4]cyclopenta[1,2-c]pyrazole-5,2′-1,3]dithiolane]-1(3bH)-yl)acetate (10 g 24.83 mmol) in DCM (20 mL) at the same temperature. The reaction mixture was allowed to warm to −40° C. and stirred for 1 h. Progress of the reaction was monitored by TLC (SiO₂, 30% EtOAc/Pet, Rf=0.3, UV in-active). After completion, the reaction mixture was neutralized with sat. NaHCO₃ (200 mL) and extracted with EtOAc (2×100 mL) at 27° C. The combined organics were washed with brine (50 ml), dried over anhydrous Na₂SO₄, filtered and evaporated under reduced pressure to get crude (15 g) as brown gum. The above crude compound was purified by column chromatography using silica gel (100-200 mesh) with 0-25% EtOAc/Pet as eluent. The fractions containing product were collected and concentrated under reduced pressure to afford ethyl 2-(3-(difluoromethyl)-5,5-difluoro-3b,4,4a,5-tetrahydro-1H-cyclopropa[3,4]cyclopenta[1,2-c]pyrazol-1-yl)acetate (8.5 g, Yield: 91%) as a pale yellow solid. ¹H NMR (400 MHz, CHLOROFORM-d) δ=6.62 (t, J=55.2 Hz, 1H), 4.82 (s, 2H), 4.30-4.18 (m, 2H), 2.51-2.37 (m, 2H), 1.42-1.35 (m, 1H), 1.31-1.23 (m, 3H), 1.14-1.08 (m, 1H). LCMS M+H=293.07.

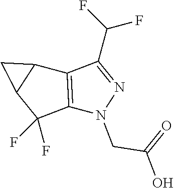 [SMILES: O=C(O)Cn1nc(C(F)F)c2c1C(F)(F)C1CC21]

### 2-(3-(difluoromethyl)-5,5-difluoro-3b,4,4a,5-tetrahydro-1H-cyclopropa[3,4]cyclopenta[1,2-c]pyrazol-1-yl)acetic acid

To a stirred solution of ethyl 2-(3-(difluoromethyl)-5,5-difluoro-3b,4,4a,5-tetrahydro-1H-cyclopropa[3,4]cyclopenta[1,2-c]pyrazol-1-yl)acetate (15 g, 49.8 mmol) in THF (17 mL) and MeOH (65.6 mL), was added LiOH (1.788 g, 74.7 mmol in water 65.6 mL) at 0° C. under N2 atmosphere. The reaction mixture was stirred for 3 h at 27° C. Progress of the reaction was monitored by TLC (SiO₂, 5% MeOH/DCM, Rf=0.2, UV Active). After completion, the reaction mixture was evaporated under reduced pressure, diluted with water (50 mL) and washed with EtOAc (2×250 mL) to remove impurities. The Aqueous layer was acidified with 1N HCl up to pH=2-3, extracted with EtOAc (3×1000 mL). The combined organics were dried over anhydrous Na₂SO₄, filtered and evaporated under reduced pressure to afford 2-(3-(difluoromethyl)-5,5-difluoro-3b,4,4a,5-tetrahydro-1H-cyclopropa[3,4]cyclopenta[1,2-c]pyrazol-1-yl)acetic acid (14 g, Yield: 98%) as an off white solid. LCMS M+H=265.15.

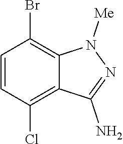 [SMILES: Cn1nc(N)c2c(Cl)ccc(Br)c21]

### 7-bromo-4-chloro-1-methyl-1H-indazol-3-amine

To a solution of 3-bromo-6-chloro-2-fluorobenzonitrile (360.0 g, 1.55 mol, 1.0 equiv.) in ethanol (1.08 L) was added methylhydrazine sulphate (1.11 kg, 7.73 mol, 5.0 equiv.) followed by the addition of triethylamine (1.3 L, 9.3 mol, 6.0 equiv.) at 25-35° C. The reaction mixture was heated to 110° C. and maintained for 15 h (the reaction was monitored by TLC). After completion of the reaction the mixture was cooled to room temperature. Water (3.0 L) was added and the mixture was stirred for 1 h at room temperature. The solids were isolated via filtration and were washed with water. The wet solid was dried under vacuum at 50° C. for 12-15 hours. The crude solid was purified by column chromatography (10% EA/hexanes to 40% EA/Hexanes) to afford the product as a pale yellow solid. Yield: 185.0 g (46.0%).

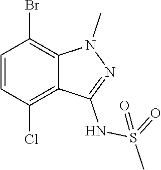 [SMILES: Cn1nc(NS(C)(=O)=O)c2c(Cl)ccc(Br)c21]

### N-(7-bromo-4-chloro-1-methyl-1H-indazol-3-yl)methanesulfonamide

To a solution of 7-bromo-4-chloro-1-methyl-1H-indazol-3-amine (1.40 g, 5.37 mmol) in DCM (30 mL) was added Hunig's Base (3.75 mL, 21.5 mmol) and then the reaction was cooled in an ice bath and methanesulfonyl chloride (1.26 mL, 16.1 mmol) was added. The reaction mixture was stirred at this temperature for 1 h (precipitate formed). Mixture was then diluted with dichloromethane (100 mL) and washed with water, 1 M HCl and brine, dried (Na₂SO₄), filtered and concentrated in vacuo. The residue was taken up in EtOH (30 ml) and 10 ml of 20% aq. NaOH. The resulted mixture heated with a heat gun until it became a homogeneous solution and stirred at rt for 30 min. The mixture was diluted with water (80 mL) and acidified with 1 N HCl (60 mL). The precipitate was filtered, washed with water, and dried in vacuo to afford the title product (1.5 g) as an off-white solid. ¹H NMR (500 MHz, CDCl₃) δ 7.48 (d, J=7.9 Hz, 1H), 7.24 (br s, 1H), 6.95 (d, J=7.9 Hz, 1H), 4.38 (s, 3H), 3.42 (s, 3H). LC/MS (M+H)⁺=337.80.

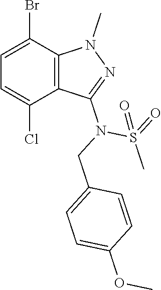 [SMILES: COc1ccc(CN(c2nn(C)c3c(Br)ccc(Cl)c23)S(C)(=O)=O)cc1]

### N-(7-bromo-4-chloro-1-methyl-1H-indazol-3-yl)-N-(4-methoxybenzyl)methanesulfonamide

To a mixture of N-(7-bromo-4-chloro-1-methyl-1H-indazol-3-yl)methanesulfonamide (1.3 g, 3.84 mmol) and 1-(chloromethyl)-4-methoxybenzene (0.625 mL, 4.61 mmol) in DMF (30 mL) was added cesium carbonate (1.626 g, 4.99 mmol) and the mixture was heated at 80° C. for 2 h. The mixture was poured into water (100 mL) and extracted with EtOAc (50 ml, 2×). The combined organic layer was washed with brine, dried over MgSO₄, filtered and concentrated in vacuo. The residue was purified by Bioateg (0˜35% EtOAc-hexanes) to afford the title product (1.5 g) as a white foam. ¹H NMR (500 MHz, CDCl₃) δ 7.44 (d, J=7.9 Hz, 1H), 7.31 (d, J=8.5 Hz, 2H), 6.99 (d, J=7.9 Hz, 1H), 6.84 (d, J=8.5 Hz, 2H), 4.99 (br s, 1H), 4.76 (br s, 1H), 4.40 (s, 3H), 3.80 (s, 3H), 3.01 (s, 3H).

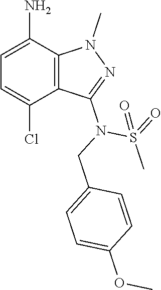 [SMILES: COc1ccc(CN(c2nn(C)c3c(N)ccc(Cl)c23)S(C)(=O)=O)cc1]

### N-(7-amino-4-chloro-1-methyl-1H-indazol-3-yl)-N-(4-methoxybenzyl)methanesulfonamide

Following the reference: Andersen, Jacob et al, Synlett 2005 (14), 2209-2213. To a mixture of N-(7-bromo-4-chloro-1-methyl-1H-indazol-3-yl)-N-(4-methoxybenzyl)methane sulfonamide (600.0 mg, 1.308 mmol), copper(I) iodide (49.8 mg, 0.262 mmol), sodium ascorbate (518 mg, 2.62 mmol) and (1R,2R)—N1,N2-dimethylcyclohexane-1,2-diamine (46.5 mg, 0.327 mmol) in NMP (10 mL) was added a solution of sodium azide (255 mg, 3.92 mmol) in Water (2.0 mL). The mixture was then sealed and heated in a microwave system at 120° C. for 2.5 h. The mixture was then filtered through a pad of Celite and the pad was washed with EtOAc. The filtrate was poured into water (100 mL) and extracted with EtOAc (50 ml, 2×). The combined organic layer was washed with brine, dried over MgSO₄, filtered and evaporated in vacuo. The residue was purified by Biotage (5-100% EtOAc/hexanes) to afford the title product (400 mg) as an off-white solid. F¹H NMR (400 MHz, CDCl₃) δ 7.33-7.29 (m, 2H), 6.89 (d, J=7.8 Hz, 1H), 6.85-6.79 (m, 2H), 6.48 (d, J=7.8 Hz, 1H), 5.11 (br.s, 1H), 4.81 (br.s, 1H), 4.30 (s, 3H), 3.80 (br s, 2H), 3.79 (s, 3H), 2.99 (s, 3H). LC/MS (M+H)⁺=395.00.

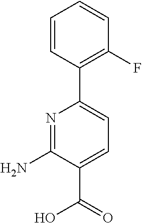 [SMILES: Nc1nc(-c2ccccc2F)ccc1C(=O)O]

### 2-amino-6-(2-fluorophenyl)nicotinic acid

A solution of 2-amino-6-chloronicotinic acid (1.0 g, 5.79 mmol), Pd(dppf)₂Cl₂ (0.21 g, 0.29 mmol), (2-fluorophenyl)boronic acid (1.14 g, 8.11 mmol), and sodium carbonate (1.23 g, 11.59 mmol) in dioxane (46 mL) and water (12 mL) was degassed and heated to 100° C. for 3 h. After cooling to ambient temperature, the reaction mixture was added to water and washed with ether. The aqueous layer was then acidified with 0.5 M citric acid. The aqueous slurry was washed with 10% IPA/DCM (×3). The aqueous layer was then filtered to provide an off-white solid of semipure product of ˜80% purity (0.87 g, 65%) which was used in the next reaction without further purification. ¹H NMR (500 MHz, DMSO-d6) δ 8.14 (br d, J=7.94 Hz, 1H), 7.90-7.97 (m, 1H), 7.48-7.54 (m, 1H), 7.31-7.37 (m, 2H), 7.04 (br d, J=8.24 Hz, 1H). LC/MS: m/z=233.15 [M+1]⁺.

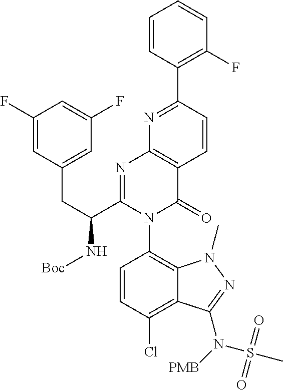

### (S)-tert-butyl (1-(3-(4-chloro-3-(N-(4-methoxybenzyl)methylsulfonamido)-1-methyl-1H-indazol-7-yl)-7-(2-fluorophenyl)-4-oxo-3,4-dihydropyrido[2,3-d]pyrimidin-2-yl)-2-(3,5-difluorophenyl)ethyl)carbamate

A mixture of (S)-2-((tert-butoxycarbonyl)amino)-3-(3,5-difluorophenyl)propanoic acid (0.908 g, 3.01 mmol), N-(7-amino-4-chloro-1-methyl-1H-indazol-3-yl)-N-(4-methoxybenzyl)methanesulfonamide (1.190 g, 3.01 mmol), and diphenyl phosphite (2.334 mL, 12.06 mmol) in pyridine (6.03 mL) in a pressure vial was heated in an aluminum block for 2 h at 75° C. and cooled to rt. The reaction diluted with EtOAc (˜150 mL) and washed with 0.5 M citric acid, dried over Na₂SO₄, and concentrated in vacuo. The residue was purified silica gel flash column chromatography (220 g isco column) using 0-50% ethyl acetate in hexanes, then 50-70% ethyl acetate in hexanes. The desired fractions were concentrated to give a yellow solid (1.37 g, 52%). LC/MS: m/z=874.25 [M+1]⁺.

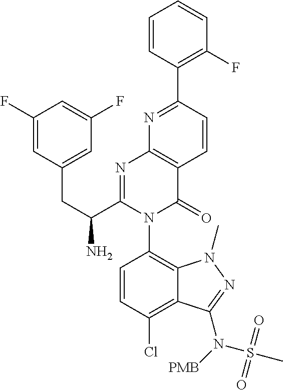

### (S)—N-(7-(2-(1-amino-2-(3,5-difluorophenyl)ethyl)-7-(2-fluorophenyl)-4-oxopyrido[2,3-d]pyrimidin-3(4H)-yl)-4-chloro-1-methyl-1H-indazol-3-yl)-N-(4-methoxybenzyl)methanesulfonamide

To a solution of tert-butyl (S)-(1-(3-(4-chloro-3-(N-(4-methoxybenzyl)methylsulfonamido)-1-methyl-1H-indazol-7-yl)-7-(2-fluorophenyl)-4-oxo-3,4-dihydropyrido[2,3-d]pyrimidin-2-yl)-2-(3,5-difluorophenyl)ethyl)carbamate (1.37 g, 1.567 mmol) in DCM (3.92 mL) was added 4 N HCl in dioxane (3.92 mL, 15.67 mmol). After stirring for 1 h, the resultant pale-yellow suspension was diluted with DCM, washed with saturated aqueous NaHCO₃, dried over Na₂SO₄, and concentrated in vacuo to give a yellow solid (1.16 g, 96%) which was carried on without further purification. LC/MS: m/z=774.20 [M+1]+.

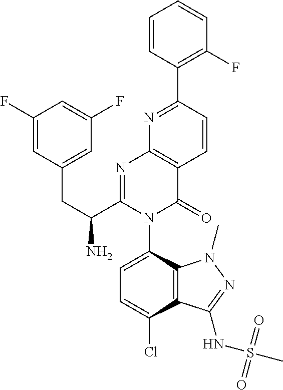

### (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-methyl-1H-indazol-3-yl)methanesulfonamide

To a solution of (S)—N-(7-(2-(1-amino-2-(3,5-difluorophenyl)ethyl)-7-(2-fluorophenyl)-4-oxopyrido[2,3-d]pyrimidin-3(4H)-yl)-4-chloro-1-methyl-1H-indazol-3-yl)-N-(4-methoxybenzyl)methanesulfonamide (1.17 g, 1.511 mmol) in DCM (7.56 ml) and TFA (4.73 mL) was added TfOH (0.403 mL, 4.53 mmol). The reaction was stirred for 1 h and then concentrated in vacuo. The residue was partitioned between 1 M NaOH and EtOAc. The EtOAc layer was dried (Na₂SO₄) and concentrated in vacuo. The crude mixture of atropisomers was purified on silica gel (120 g isco column) using 20-100% ethyl acetate in hexanes. The desired fractions were concentrated to give an off-white solid. This solid was further purified on a 275 g C18 column using 10-60% 95:5 CH₃CN:Water with 0.1% TFA in 95:5 Water:Acetonitrile with 0.1% TFA to separate the atropisomers. The second (major) eluting peak was concentrated (220 mg). The major atropisomer was chirally purified by SFC chromatography on a Chiralpak ID, 25 mm×250 mm, 5 u column, using a A:B gradient, solvent A 80% heptane, 0.1% TFA solvent B 20% ethanol, 0.1% TFA to provide the desired product (176 mg, 18%, chiral purity 98.2%). LC/MS: m/z=654.15 [M+1]⁺.

### Example 1

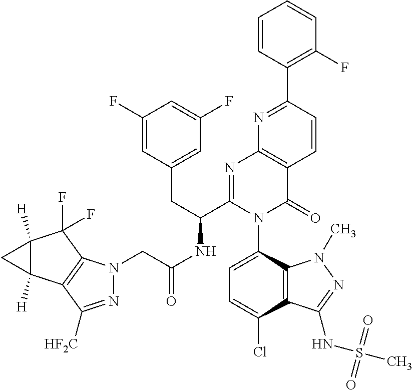

### N—((S)-1-((3P)-3-(4-chloro-1-methyl-3-(methylsulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

To a stirred solution of (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-methyl-1H-indazol-3-yl)methanesulfonamide (0.03 g, 0.039 mmol) in DMF (1 mL) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (11 mg, 0.041 mmol), 2-(3H-[1,2,3]triazolo[4,5-b]pyridin-3-yl)-1,1,3,3-tetramethylisouronium hexafluorophosphate(V) (0.018 g, 0.047 mmol), and DIPEA (0.024 mL, 0.137 mmol). After 1 h, the reaction mixture was purified directly by preparative HPLC. Purification Conditions: Column: Zorbax Eclipse Plus C18, 21.2×100 mm, 5 m particles; Solvent A=0.1% Formic Acid in 100% Water. Solvent B=Acetonitrile. Flow Rate=40 mL/min. Start % B=55 Final % B=75. Gradient Time=7 min, then a 2 min hold at 98% B. Wavelength=215 and 254 nm. ESI+Range: 150 to 1500 dalton. The product was isolated. (0.0143 g, 28%). 1H NMR (500 MHz, METHANOL-d4) δ 8.73-8.81 (m, 1H) 8.05-8.15 (m, 2H) 7.58-7.68 (m, 1H) 7.25-7.49 (m, 4H) 6.52-6.85 (m, 4H) 4.89-4.93 (m, 1H) 4.54-4.64 (m, 2H) 3.67 (s, 3H) 3.49-3.55 (m, 1H) 3.34-3.36 (m, 1H) 3.26 (s, 3H) 3.12-3.20 (m, 1H) 2.38-2.47 (m, 2H) 1.33-1.40 (m, 1H) 0.98-1.04 (m, 1H). LC/MS retention time=1.39 min; m/z=899.2 [M]+ (Column: Acquity UPLC BEH C18, 2.1×30 mm, 1.7 m particles; Solvent A=0.1% Formic acid in 100% Water. Solvent B=0.1% Formic Acid in 100% Acetonitrile. Flow Rate=0.8 mL/min. Start % B=5. Final % B=95. Gradient Time=1.6 min, then a 0.25 min hold at 95% B. Wavelength=215 nm).

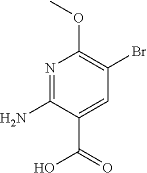

### 2-amino-5-bromo-6-methoxynicotinic acid

To a slurry of 2-amino-6-methoxynicotinic acid (3.0 g, 17.84 mmol) in AcOH (60 mL) was added NBS (3.81 g, 21.41 mmol). After 20 min, the mixture was added into water (500 mL) and the resultant precipitate was filtered to provide the product (2.85 g, 91%). ¹H NMR (500 MHz, DMSO-d₆) δ 12.73 (s, 1H), 8.05 (s, 1H), 7.43 (br s, 1H), 3.90 (s, 3H). LC/MS: m/z=247.0 [M+1]⁺.

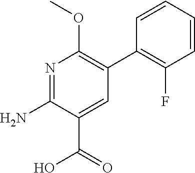 [SMILES: COc1nc(N)c(C(=O)O)cc1-c1ccccc1F]

### 2-amino-5-(2-fluorophenyl)-6-methoxynicotinic acid

A solution of 2-amino-5-bromo-6-methoxynicotinic acid (1.0 g, 4.05 mmol), (2-fluorophenyl)boronic acid (0.79 g, 5.67 mmol), sodium carbonate (0.86 g, 8.10 mmol), and Pd(dppf)₂Cl₂ (0.15 g, 0.202 mmol) in degassed dioxane (32 mL) and water (8 mL) was heated to 100° C. for 3 h. Upon cooling to ambient temperature, the reaction was added to water and washed with ether (×2). The aqueous layer was acidified with 0.5 M citric acid and filtered. The crude product was purified by flash column silica gel chromatography (20-100% EtOAc/hex with 1% AcOH) to provide semipure product (0.60 g). This material was further purified by C18 reverse phase chromatography (10-60% MeCN/water, 0.1% TFA) to provide the product (0.36 g, 34%) white solid. ¹H NMR (500 MHz, DMSO-d₆) δ 12.55 (br s, 1H), 7.87 (s, 1H), 7.43 (br s, 2H), 7.33-7.40 (m, 2H), 7.22 (br t, J=7.78 Hz, 2H). LC/MS: m/z=263.15 [M+1]⁺.

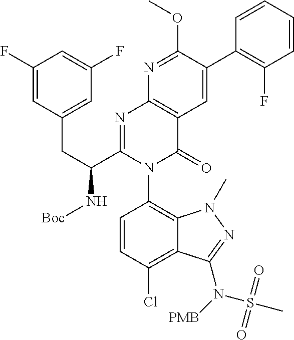

### (S)-tert-butyl (1-(3-(4-chloro-3-(N-(4-methoxybenzyl)methylsulfonamido)-1-methyl-1H-indazol-7-yl)-6-(2-fluorophenyl)-7-methoxy-4-oxo-3,4-dihydropyrido[2,3-d]pyrimidin-2-yl)-2-(3,5-difluorophenyl)ethyl)carbamate

A mixture of (S)-2-((tert-butoxycarbonyl)amino)-3-(3,5-difluorophenyl)propanoic acid (0.414 g, 1.373 mmol), 2-amino-5-(2-fluorophenyl)-6-methoxynicotinic acid (0.36 g, 1.373 mmol), and diphenyl phosphite (1.063 ml, 5.49 mmol) in pyridine (2.75 ml) was stirred for 1 h. N-(7-amino-4-chloro-1-methyl-1H-indazol-3-yl)-N-(4-methoxybenzyl)methanesulfonamide (0.542 g, 1.373 mmol) was then added and the reaction was heated at to 75° C. for 2.5 h. Upon cooling to ambient temperature, the reaction was concentrated in vacuo. The crude product was purified by silica gel chromatography (220 g isco column) using 0-60% ethyl acetate in hexanes to give the product (0.54 g, 44%) as a pale yellow solid. LC/MS: m/z=904.25 [M+1]⁺.

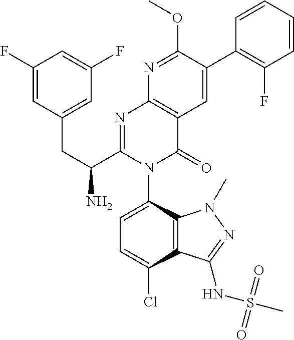

### (S)—N-((6P)-7-(2-(1-amino-2-(3,5-difluorophenyl)ethyl)-6-(2-fluorophenyl)-7-methoxy-4-oxopyrido[2,3-d]pyrimidin-3(4H)-yl)-4-chloro-1-methyl-1H-indazol-3-yl)methanesulfonamide

To a solution of (S)-tert-butyl (1-(3-(4-chloro-3-(N-(4-methoxybenzyl)methylsulfonamido)-1-methyl-1H-indazol-7-yl)-6-(2-fluorophenyl)-7-methoxy-4-oxo-3,4-dihydropyrido[2,3-d]pyrimidin-2-yl)-2-(3,5-difluorophenyl)ethyl)carbamate (0.54 g, 0.597 mmol) in DCM (2.99 ml) and TFA (1.867 ml)) was added TfOH (0.053 ml, 0.597 mmol). The mixture was stirred for 1 h and concentrated in vacuo. The residue was partitioned between ethyl acetate and 1 M NaOH. The EtOAc layer was dried over Na₂SO₄ and concentrated in vacuo. The crude product was purified on silica gel (80 g isco column) using 20-80% ethyl acetate in hexanes to give an off-white solid (0.257 g), first eluting/major atropisomer. This product was chirally purified by SFC chromatography: solvent A: (70%) of heptane, solvent B: (30%) of ethanol, column: ChiralPak ID (25×250 mm, 5 micron) Flow 45 ml/min, Wavelength −214, 220 nm Temp Ambient. ¹H NMR (500 MHz, DMSO-d6) δ 8.16-8.30 (m, 1H) 7.20-7.53 (m, 6H) 6.87-7.05 (m, 1H) 6.60-6.71 (m, 2H) 3.95-4.04 (m, 3H) 3.59-3.71 (m, 3H) 3.42-3.50 (m, 1H) 3.17-3.23 (m, 1H) 3.11-3.16 (m, 3H) 2.77-2.84 (m, 1H). LC/MS: m/z=684.20 [M+1]⁺.

### Example 2

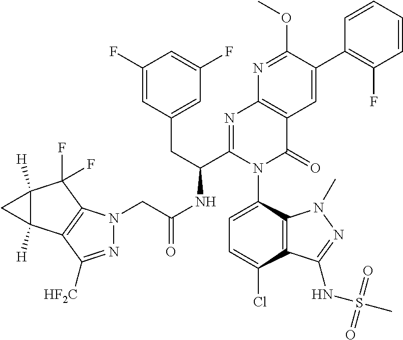

### N—((S)-1-((3P)-3-(4-chloro-1-methyl-3-(methylsulfonamido)-1H-indazol-7-yl)-6-(2-fluorophenyl)-7-methoxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

To a stirred solution of (S)—N-((6P)-7-(2-(1-amino-2-(3,5-difluorophenyl)ethyl)-6-(2-fluorophenyl)-7-methoxy-4-oxopyrido[2,3-d]pyrimidin-3(4H)-yl)-4-chloro-1-methyl-1H-indazol-3-yl)methanesulfonamide (0.05 g, 0.073 mmol) in DMF (0.731 mL) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.019 g, 0.073 mmol), 2-(3H-[1,2,3]triazolo[4,5-b]pyridin-3-yl)-1,1,3,3-tetramethylisouronium hexafluorophosphate(V) (0.028 g, 0.073 mmol), and DIPEA (0.013 mL, 0.073 mmol). The reaction mixture was stirred for 2, diluted with DMF, filtered and then purified directly by preparatory HPLC. Purification Conditions: Column: Waters Xterra C18, 19×100 mm, 10 m particles; Solvent A=0.1% NH4OH in 100% Water. Solvent B=Acetonitrile. Flow Rate=40 mL/min. Start % B=56. Final % B=76. Gradient Time=6 min, then a 2 min hold at 98% B. Wavelength=215 and 254 nm. ESI+Range: 150 to 1500 dalton. The product was isolated (0.040 g, 59%). ¹H NMR (500 MHz, METHANOL-d₄) δ 8.42-8.46 (m, 1H) 7.45-7.54 (m, 2H) 7.21-7.34 (m, 4H) 6.56-6.84 (m, 4H) 4.88-4.92 (m, 2H) 4.53-4.64 (m, 2H) 4.15-4.19 (m, 3H) 3.64-3.68 (m, 3H) 3.48-3.54 (m, 1H) 3.23-3.27 (m, 3H) 3.12-3.20 (m, 1H) 2.40-2.49 (m, 2H) 1.34-1.41 (m, 1H) 1.00-1.05 (m, 1H). LC/MS retention time=1.42 min; m/z=930.7 [M+H]⁺ (Column: Acquity UPLC BEH C18, 2.1×30 mm, 1.7 m particles; Solvent A=0.1% Formic acid in 100% Water. Solvent B=0.1% Formic Acid in 100% Acetonitrile. Flow Rate=0.8 mL/min. Start % B=5. Final % B=95. Gradient Time=1.6 min, then a 0.25 min hold at 95% B. Wavelength=215 nm.

### Alternate preparation of N-(7-amino-4-chloro-1-methyl-1H-indazol-3-yl)-N-(4-methoxybenzyl)methanesulfonamide

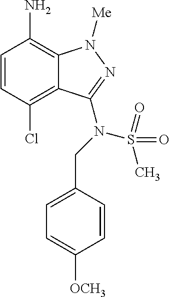 [SMILES: COc1ccc(CN(c2nn(C)c3c(N)ccc(Cl)c23)S(C)(=O)=O)cc1]

### Synthesis Scheme:

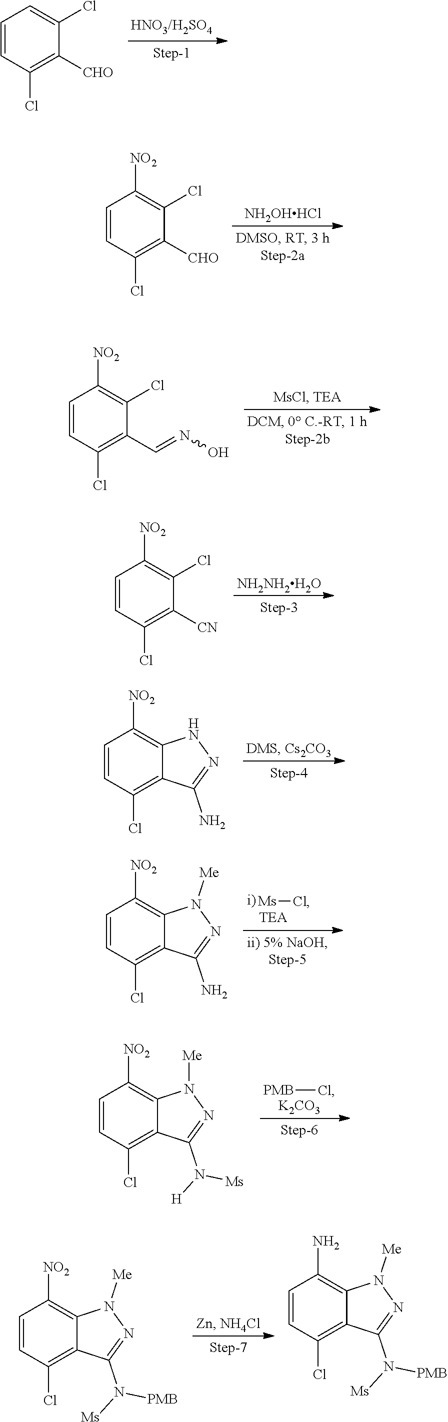

### Step 1: Preparation of 2,6-dichloro-3-nitrobenzaldehyde

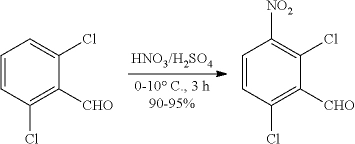 [SMILES: O=Cc1c(Cl)cccc1Cl ; O=Cc1c(Cl)ccc([N+](=O)[O-])c1Cl]

To a solution of sulfuric acid (H₂SO₄) (5.63 L, 4.5 V) in a round-bottom flask at 0-5° C. was added 2,6-dichlorobenzaldehyde (1.25 kg, 7.10 mol, 1.0 equiv.) in portions at below 15° C. The reaction mass was stirred at 0-5° C. for 30 min. A solution of freshly prepared nitration mixture [Prepared from Conc. H₂SO₄ (0.425 L, 0.34 V) and 70% HNO₃ (0.85 kg, 13.49 mol, 1.30 equiv.) at 0° C.] was added to the above reaction mixture at below 10° C. [Note: Reaction is slightly exothermic (3-6° C.); so that addition is preferred at lower temperature]. The reaction mixture was stirred at 5-10° C. for 2-3 h. After completion of the reaction (monitored by TLC), it was quenched with ice cold water (18.75 L, 15 V) at below 25° C. Then the reaction mass was allowed warm to room temperature and stirred for 2 h. The solids were isolated by filtration and then were washed with water (2.5 L, 2.0 V). Bulk residual water was removed from the solids by maintaining vacuum filtration for 60-90 min. The crude wet solid was initially dried under air atmosphere; then in a hot air oven at 50-55° C. for 10-12 h (until moisture content is not more than 5.0%) to get the dried title product, 2,6-dichloro-3-nitrobenzaldehyde (1.44 kg, 92% yield) as a yellow solid. ¹H NMR (400 MHz, CDCl₃): δ 10.44 (s, 1H), 7.88 (d, J=8.4 Hz, 1H), 7.56 (d, J=8.8 Hz, 1H).

### Step 2: Preparation of 2,6-dichloro-3-nitrobenzonitrile

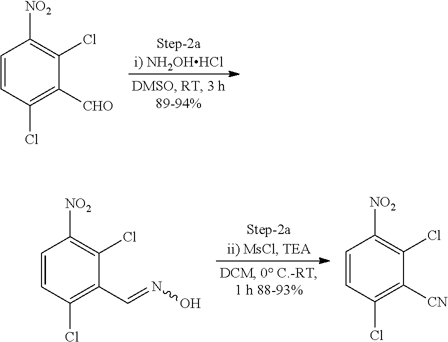

(Step-2a) To a solution of DMSO (5.9 L, 5.0 V)) in a round-bottom flask was added 2,6-dichloro-3-nitrobenzaldehyde (1.17 kg, 5.31 mol, 1.0 equiv.) at room temperature. After being stirred for 30 min at room temperature, hydroxylamine hydrochloride (0.63 kg, 9.04 mol, 1.70 equiv.) was added and the reaction mass was stirred at room temperature for 3 h. After completion of the reaction (monitored by TLC), the reaction mass was quenched by the addition of ice cold water (18.0 L, 15.0 V) added at a rate sufficient to maintain the temperature below 30° C. (Observation: Solids will formed upon water addition). The reaction mass was stirred at room temperature for 60-90 min. The solids were isolated by filtration; washed with water (2.5 L, 2.0 V); followed by washing with a mixture of acetone and hexanes (6.0 L, 1:1 ratio). Bulk residual water was removed from the solids by maintaining vacuum filtration for 60-90 min. The wet solid was initially air dried and then finally dried in a hot air oven at 50-55° C. for 10-12 h (until moisture content was not more than 1.0%) to get the dried target product, 2,6-dichloro-3-nitrobenzaldehyde oxime (1.22 kg, 92% yield) as an off-white solid. The crude product (which contains 10-20% of 2,6-dichloro-3-nitrobenzonitrile) was used directly in the next step without further purification.
(Step-2b) To a stirred solution of the crude oxime (preparation described above, 1.13 kg, 4.80 mol, 1.0 equiv.) in DCM (9.04 L, 8.0 V) at 0-5° C. was added triethylamine (“TEA”, 1.02 kg, 10.09 mol, 2.1 equiv.). After being stirred for 5 min, methanesulfonyl chloride (0.60 kg, 5.29 mol, 1.1 equiv.) was added (Observation: An exotherm is noted during the addition) slowly at 15° C. Then the reaction mass was stirred at room temperature for 30-45 min. After completion of the reaction (progress of reaction was monitored by TLC; mobile phase: 20% ethyl acetate in hexanes), the reaction mass was diluted with water (6.78 L, 6.0 V); the organic layer was separated; and the aqueous layer was extracted with DCM (3.4 L, 3.0 V). The combined organic layers were washed with brine (5.65 L, 5.0 V); dried over Na₂SO₄; and concentrated under vacuum. The resulting crude solids were triturated with hexanes (4.50 L, 4.0 V) at room temperature. The wet material was dried in a hot air oven at 50-55° C. for 5-6 h to get the dried product, 2,6-dichloro-3-nitrobenzonitrile (0.95 kg, 91% yield) as a yellow solid. ¹H NMR (400 MHz, CDCl₃): δ 8.07 (d, J=8.8 Hz, 1H), 7.63 (d, J=8.8 Hz, 1H).

### Step 3: Preparation of 4-chloro-7-nitro-1H-indazol-3-amine

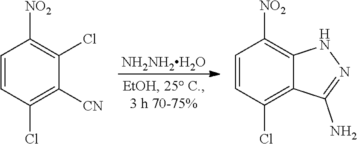

To a stirred solution of 2,6-dichloro-3-nitrobenzonitrile (750.0 g, 3.45 mol, 1.0 equiv.) in ethanol (7.5 L, 10.0 V) at 15-20° C. was slowly added hydrazine hydrate (519.0 g, 10.36 mol, 3.0 equiv.) while maintaining the reaction mass below 25° C. (Observation: Addition is slightly exothermic and solid formation will begin upon addition). The reaction mixture temperature was slowly raised to room temperature and then the mixture was stirred for 3 h (Observation: the quantity of solids will increase during this time). After completion of the reaction (monitored by TLC), the mixture was diluted with water (7.5 L, 10.0 V) and further stirred for 1 h at room temperature. The solids were isolated via filtration and then were washed with water (2.25 L, 3.0 V). The wet solid was washed with a 1:1 ratio mixture of acetone (1.875 L, 2.5 V) and hexanes (1.875 L, 2.5 V). Bulk residual water was removed from the solids by maintaining vacuum filtration for 60-90 min. The wet solid was finally dried in a hot air oven for 7-8 h at 50° C. (until moisture content reaches below 1.5%) to get the dried product, 4-chloro-7-nitro-1H-indazol-3-amine (549.0 g, 75% yield) as a brick red-colored solid. ¹H NMR (400 MHz, CDCl₃): δ 10.36 (bs, 1H), 8.20 (d, J=8.4 Hz, 1H), 7.07 (d, J=8.40 Hz, 1H), 4.73 (bs, 2H).

### Step 4: Preparation of 4-chloro-1-methyl-7-nitro-1H-indazol-3-amine

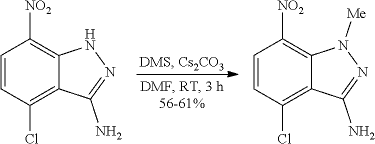

To a stirred solution of 4-chloro-7-nitro-1H-indazol-3-amine (500 g, 0.42 mol, 1.0 equiv.) in DMF (5.0 L, 10.0 V) at 5-10° C. was slowly added cesium carbonate (Cs₂CO₃) (1.91 kg, 5.88 mol, 2.5 equiv.) while maintaining the reaction mass below 10° C. After being stirred for 5-10 min, dimethyl sulphate (326.3 g, 2.59 mol, 1.1 equiv.) was added while maintaining the reaction mass below 10° C. (Note: Slow addition is preferred for obtaining more favorable regio-selectivity). Then, the reaction temperature was slowly raised to room temperature and stirring was continued an additional 2 h at the same temperature. After completion of the reaction (monitored by TLC), the reaction mass was quenched by the addition of ice-cold water (15.0 L, 30.0 V) and the resulting mixture was then stirred for 6-8 h at room temperature. The solids were isolated via filtration and were then washed with water (1.5 L, 3.0 V). The wet solid was washed with IPA (1.5 L, 3.0 V) followed by hexanes (1.0 L, 2.0 V). Bulk residual water was removed from the solids by maintaining vacuum filtration for 60-90 min. The wet solid was dried in a hot air oven for 7-8 h at 50° C. (until moisture content is below 1.0%). The isolated material, 4-chloro-1-methyl-7-nitro-1H-indazol-3-amine (319.0 g, 60% yield), was used in the next step without further purification. ¹H NMR (400 MHz, CDCl₃): δ 7.97 (d, J=8.32 Hz, 1H), 6.97 (d, J=8.24 Hz, 1H), 4.63 (bs, 2H), 3.96 (s, 3H).

### Step 5: Preparation of N-(4-chloro-1-methyl-7-nitro-1H-indazol-3-yl)methanesulfonamide

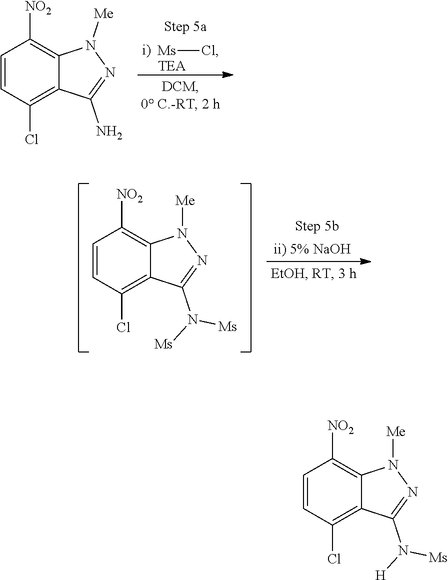

(Step 5a) To a solution of 4-chloro-1-methyl-7-nitro-1H-indazol-3-amine (625.0 g, 2.76 mol, 1.0 equiv.) in DCM (6.25 L, 10.0 V) at 0-5° C. was added triethylamine (TEA) (837.0 g, 8.27 mol, 3.0 equiv.); followed by the addition of 4-dimethylaminopyridine (DMAP) (20.60 g, 0.165 mol, 0.06 equiv.). The reaction mass was stirred for 5-10 min., then methanesulfonyl chloride (MsCl) (790.0 g, 6.89 mol, 2.5 equiv.) added slowly while maintaining the reaction mass below 10° C. The reaction mixture was allowed to warm to room temperature and was then stirred for 1.5-2.0 h. After completion of the reaction (monitored by TLC), the mixture was diluted with water (6.25 L, 10.0 V) and then stirred at room temperature for 15 min. The organic layer was separated, and the aqueous layer was extracted with DCM (6.25 L, 10.0 V). The combined organic layers were washed with brine (1.25 L, 2.0 V), dried over Na₂SO₄ and concentrated to get the crude solids. The solids were triturated with hexanes (1.25 L, 2.0 V) at room temperature to obtain the intermediate, N-(4-chloro-1-methyl-7-nitro-1H-indazol-3-yl)-N-(methylsulfonyl)methanesulfonamide, which was used directly in the next step. (ii) To a stirred solution of N-(4-chloro-1-methyl-7-nitro-1H-indazol-3-yl)-N-(methylsulfonyl)methanesulfonamide (prepared above) in ethanol (10.5 L, 20.0 V) at room temperature was added slowly an aq. 5% NaOH solution (4.38 L, 7.0 V) [Note: Slow addition is preferred via dropping funnel]. The reaction mass was stirred at the same temperature for 3 h. After completion of the reaction (monitored by TLC) [Sample preparation for TLC analysis: ˜1.0 ml of sample acidified with aq. 2.0 N HCl to reach the pH: 2-3, extract it with ethyl acetate and analyze the organic layer by TLC], the reaction mass was cooled to 0-5° C. and the pH was adjusted to 2-3 by the addition of aq. 2.0 N HCl (3.13 L, 5.0 V) while maintain the reaction temperature below 10° C. [Note: Precipitation occurred upon addition of HCl and increased with stirring]. The reaction mixture was warmed to room temperature and then stirred for 1.5-2.0 h. Solids obtained were isolated via filtration and were then washed with water (1.25 L, 2.0 V); followed by washing with hexanes (1.25 L, 2.0 V). Bulk residual water was removed from the solids by maintaining vacuum filtration for 60-90 min. The wet material was dried in a hot air oven at 50° C. for 6-7 h (Until the moisture content is below 1.0%) to get the dried product, N-(4-chloro-1-methyl-7-nitro-1H-indazol-3-yl)methanesulfonamide (640.0 g, 76%) as a yellow solid. ¹H NMR (400 MHz, CDCl₃): δ 8.05 (d, J=8.32 Hz, 1H), 7.32 (bs, 1H), 7.17 (d, J=8.28 Hz, 1H), 4.15 (s, 3H), 3.45 (s, 3H).

### Step 6: Preparation of N-(4-chloro-1-methyl-7-nitro-1H-indazol-3-yl)-N-(4-methoxybenzyl)methanesulfonamide

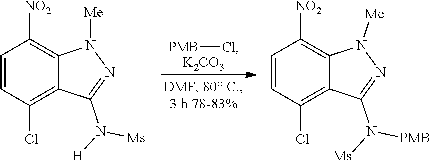

To a mixture of N-(4-chloro-1-methyl-7-nitro-1H-indazol-3-yl)methanesulfonamide (635.0 g, 2.08 mol, 1.0 equiv.) and 1-(chloromethyl)-4-methoxybenzene (359.0 g, 2.30 mol, 1.1 equiv.) in DMF (6.35 L, 10.0 V) at room temperature was added potassium carbonate (374.7 g, 2.70 mol, 1.3 equiv.). The reaction mixture was heated to 80-90° C. and maintained at that temperature for 3 h. After completion of the reaction (monitored by TLC), the mixture was poured into ice cold water (19.05 L, 30.0 V) [Note: Slow quenching with vigorous stirring is preferred to avoid clumping as the product precipitates]. The resulting solids were isolated via filtration and washed with water (1.90 L, 3.0 V); then the solids were washed with hexanes (1.27 L, 2.0 V). Bulk residual water was removed from the solids by maintaining vacuum filtration for 60-90 min. The isolated solid was dissolved in Ethyl acetate (12.7 L, 20.0 V) and charcoal was added (63.5 g). The mixture was heated to 60-70° C. and then stirred for 30-45 min. at that temperature. The mixture was filtered while still hot (40-50° C.) through a pad of Celite and the Celite pad was then extracted with ethyl acetate (3.17 L, 5.0 V). The combined filtrates were concentrated to dryness under reduced pressure at below 50° C. Ethyl acetate (0.635 L, 1.0 V) was added to the solids at room temperature. The resultant solid suspension was stirred for 30 min. The solids were isolated via filtration and then were washed with hexanes (1.27 L, 2.0 V). Residual water was removed from the solids by maintaining vacuum filtration for 45-60 min. to afford the product N-(4-chloro-1-methyl-7-nitro-1H-indazol-3-yl)-N-(4-methoxybenzyl) methane sulfonamide (705.0 g, 80% yield) as a yellow solid. ¹H NMR (400 MHz, CDCl₃): δ 7.99 (d, J=8.24 Hz, 1H), 7.27 (d, J=8.68 Hz, 2H), 7.19 (d, J=8.24 Hz, 1H), 6.80 (d, J=8.44 Hz, 2H), 4.95-4.76 (m, 2H), 4.17 (s, 3H), 3.76 (s, 3H), 3.01 (s, 3H).

### Step 7: Preparation of N-(7-Amino-4-chloro-1-methyl-1H-indazol-3-yl)-N-(4-methoxybenzyl)methanesulfonamide

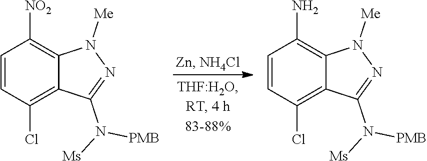

To a stirred suspension of zinc powder (540.0 g, 8.23 mol, 10.0 equiv.) in a mixture of THF (3.50 L, 10.0 V) and water (7.0 L, 20.0 V) at room temperature was added ammonium chloride (NH₄Cl) (449.0 g, 8.23 mol, 10.0 equiv.). To the mixture was added N-(4-chloro-1-methyl-7-nitro-1H-indazol-3-yl)-N-(4-methoxybenzyl)methanesulfonamide (350 g, 0.823 mol, 1.0 equiv.) in THF (7.0 L, 20.0 V). The reaction mixture was stirred at room temperature for 3-4 h. After completion of the reaction (monitored by in-process TLC/HPLC), the mixture was diluted with ethyl acetate (3.5 L, 10.0 V) and water (1.12 L, 2.5 V). The mixture was stirred for 15 min. The reaction mass was filtered through a pad of Celite bed washing with ethyl acetate (1.75 L, 5.0 V). The bi-phasic filtrate was collected, and the phases were separated. The aqueous layer was extracted with ethyl acetate (3.50 L, 10.0 V). The combined organic layers were washed with brine (3.50 L, 10 V), dried over Na₂SO₄, and then concentrated in vacuo to afford a crude solid. To the crude product was added MTBE (3.25 L, 10 V) and the suspension was stirred for 30 min at room temperature. The solids were isolated by filtration. Bulk residual water was removed from the solids by maintaining vacuum filtration for 30-45 min. The wet product was dried in a hot air oven (50° C.) for 2 h to afford the title product, N-(7-amino-4-chloro-1-methyl-1H-indazol-3-yl)-N-(4-methoxybenzyl)methanesulfonamide (276.0 g, 85% yield) as off-white solid. ¹H NMR (400 MHz, CDCl₃): δ 7.29-7.26 (m, 2H), 6.86-6.79 (m, 2H), 6.42 (d, J=7.80 Hz, 1H), 4.99-4.70 (m, 2H), 4.25 (s, 3H), 3.77 (s, 5H), 2.98 (s, 3H).

### Preparation of N-(7-amino-4-chloro-1-(2,2-difluoroethyl)-1H-indazol-3-yl)-N-(4-methoxybenzyl)methanesulfonamide

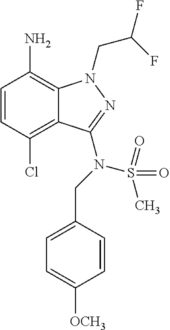 [SMILES: COc1ccc(CN(c2nn(CC(F)F)c3c(N)ccc(Cl)c23)S(C)(=O)=O)cc1]

### Synthesis Scheme:

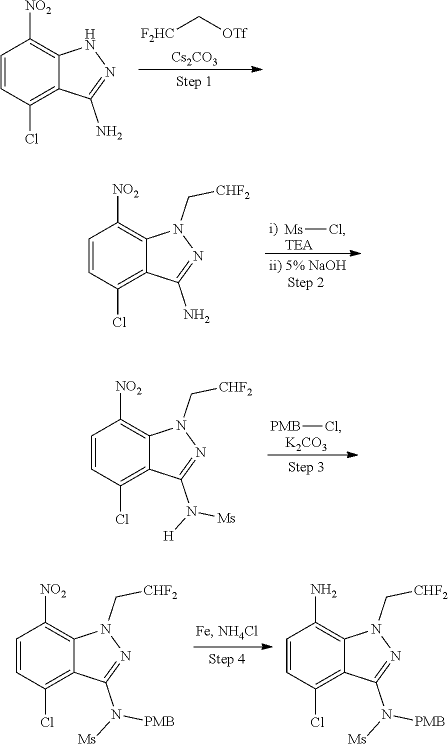

### Step 1: Preparation of 4-chloro-1-(2,2-difluoroethyl)-7-nitro-1H-indazol-3-amine

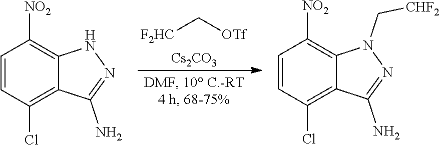

To a stirred solution of 4-chloro-7-nitro-1H-indazol-3-amine (180 g, 0.85 mol, 1.0 equiv.) in DMF (1.8 L, 10.0 V) at 10-15° C. was added cesium carbonate (Cs₂CO₃) (551 g, 1.70 mol, 2.0 equiv.) at a rate necessary to maintaining the reaction mass below 20° C. The mixture was stirred for 5-10 min, then to the stirred mixture at 10-15° C. was added 2,2-difluoroethyl trifluoromethanesulfonate (133 mL, 0.93 mol, 1.1 equiv.) at a rate necessary to maintain the reaction mass below 20° C. (Note: Slow addition is preferred to obtain more favorable regio-selectivity). The reaction mass was allowed to slowly warm to room temperature and was then stirred at the same temperature for 3 h. After completion of the reaction (monitored by TLC), the reaction mass was quenched by the addition of ice-cold water (5.4 L, 30.0 V) and the resulting mixture was allowed to warm to room temperature with stirring for 6-8 h. The solids were isolated via filtration and were then washed with water (540 mL, 3.0 V). The wet solid was washed with hexanes (0.9 L, 5.0 V). Bulk residual water was removed from the solids by maintaining vacuum filtration for 60-90 min. The wet solid was dried in a hot air oven for 7-8 h at 50° C. (until the moisture content was below 1.0%). The isolated material, 4-chloro-1-(2,2-difluoroethyl)-7-nitro-1H-indazol-3-amine (160 g, 71% yield), was used in the next step without further purification. ¹H NMR (400 MHz, CDCl₃): δ 8.05 (d, J=8.4 Hz, 1H), 7.07 (d, J=8.4 Hz, 1H), 6.00 (tt, J₁=3.9 Hz, J₂=7.7 Hz, 1H), 4.76-4.84 (m, 4H).

### Step 2: Preparation of N-(4-chloro-1-(2,2-difluoroethyl)-7-nitro-1H-indazol-3-yl)methane sulfonamide

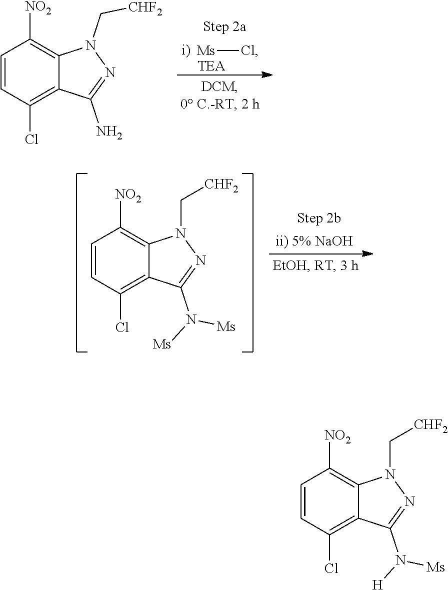

Step 2a: To a solution of 4-chloro-1-(2,2-difluoroethyl)-7-nitro-1H-indazol-3-amine (170.0 g, 0.96 mol, 1.0 equiv.) in DCM (1.7 L, 10.0 V) at 0-5° C. was added triethyl amine (264 mL, 2.88 mol, 3.0 equiv.), followed by 4-dimethylaminopyridine (3.4 g, 0.048 mol, 0.05 equiv.). The reaction mass was stirred for 5-10 min., then methanesulfonyl chloride (120 mL, 2.4 mol, 2.5 equiv.) was added slowly while maintaining the reaction mass below 10° C. The reaction mixture was allowed to warm to room temperature and then was stirred for 1.5-2.0 h. After completion of the reaction (monitored by TLC), the mixture was diluted with water (1.7 L, 10.0 V) and then stirred at room temperature for 15 min. The organic layer was separated, and the aqueous layer was extracted with DCM (1.7 L, 10.0 V). The combined organic layers were washed with 10% brine solution (340 mL, 2.0 V), dried over Na₂SO₄ and concentrated to afford the product as a crude solid. The solids were triturated with hexanes (340 mL, 2.0 V) at room temperature to obtain N-(4-chloro-1-(2,2-difluoroethyl)-7-nitro-1H-indazol-3-yl)-N-(methylsulfonyl) methanesulfonamide which was used directly in the next step.

Step 2b: To a stirred solution of N-(4-chloro-1-(2,2-difluoroethyl)-7-nitro-1H-indazol-3-yl)-N-(methylsulfonyl) methanesulfonamide (entirety of material prepared above) in ethanol (1.7 L, 10.0 V) at room temperature was added slowly aq. 5% NaOH solution (1.19 L, 7.0 V) [Note: Slow addition is preferred via dropping funnel]. The reaction mass was stirred at the same temperature for 3 h. After completion of the reaction [Sample preparation for TLC analysis: an aliquot of reaction solution (˜1 mL) was acidified with aq. 2.0 N HCl to reach the pH 2-3; then the mixture was extracted with ethyl acetate and organic layer was analyzed by TLC], the reaction mass was cooled to 0-5° C. and the pH was adjusted to 2-3 by the addition of aq. 2.0 N HCl (˜850 mL, 5.0 V) at below 10° C. [Note: Precipitation occurred upon addition of HCl and the solids increased gradually with stirring]. The reaction mixture was warmed to room temperature and then stirred for 1.5-2.0 h. Solids obtained were isolated via filtration and were then washed with water (340 mL, 2.0 V); followed by washing with hexanes (340 mL, 2.0 V). Bulk residual water was removed from the solids by maintaining vacuum filtration for 60-90 min. The wet material was dried in a hot air oven at 50° C. for 6-7 h (until the moisture content was below 1.0%) to afford the dried product, N-(4-chloro-1-(2,2-difluoroethyl)-7-nitro-1H-indazol-3-yl)methanesulfonamide (170.0 g, 75%) as a yellow solid. ¹H NMR (400 MHz, CDCl₃): δ 8.15 (d, J=8.3 Hz, 1H), 7.52 (bs, 1H), 7.24 (d, J=8.3 Hz, 1H), 6.04 (tt, J₁=3.7 Hz, J₂=7.9 Hz, 1H), 5.02 (td, J₁=3.9 Hz, J₂=14.3 Hz, 2H), 3.42 (s, 4H).

### Step 3: Preparation of N-(4-chloro-1-(2,2-difluoroethyl)-7-nitro-1H-indazol-3-yl)-N-(4-methoxy benzyl)methanesulfonamide

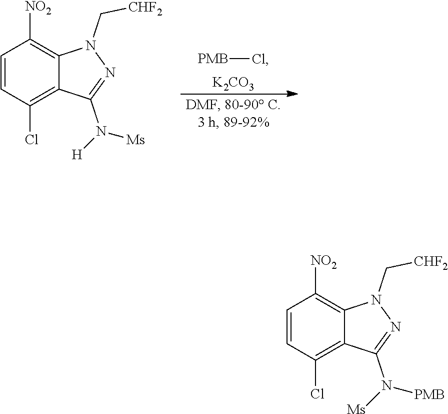

To a mixture of N-(4-chloro-1-(2,2-difluoroethyl)-7-nitro-1H-indazol-3-yl)methane sulfonamide (160.0 g, 0.45 mol, 1.0 equiv.) and 1-(chloromethyl)-4-methoxybenzene (67.6 mL, 0.5 mol, 1.1 equiv.) in DMF (1.6 L, 10.0 V) at room temperature was added potassium carbonate (93.8 g, 0.59 mol, 1.3 equiv.). The reaction mixture was heated to 80-90° C. and maintained at the same temperature for 3 h. After completion of the reaction (monitored by TLC), the mixture was poured into ice cold water (4.8 L, 60.0 V) [Note: Slow quenching with vigorous stirring is preferred to avoid clumping as the product precipitates]. The resulting solids were isolated via filtration and washed with water (480 mL, 3.0 V); then the solids were washed with hexanes (320 mL, 2.0 V). Bulk residual water was removed from the solids by maintaining vacuum filtration for 1-2 h. The isolated solid was dissolved in ethyl acetate (1.6 L, 10.0 V) and charcoal was added (16.0 g). The mixture was heated to 60-70° C. and then stirred for 30-45 min. at that temperature. The mixture was filtered while hot (40-50° C.) through a pad of Celite and the Celite pad was then extracted with ethyl acetate (800 mL, 5.0 V). The combined filtrates were concentrated to dryness under reduced pressure at below 50° C. To the resulting solids at room temperature was added ethyl acetate (160 mL, 1.0 V). The suspension was stirred for 30 min. The solids were isolated via filtration and then were washed with hexanes (320 mL, 2.0 V). Residual water was removed from the solids by maintaining vacuum filtration for 45-60 min. to afford the product N-(4-chloro-1-(2,2-difluoroethyl)-7-nitro-1H-indazol-3-yl)-N-(4-methoxybenzyl)methanesulfonamide (180.0 g, 92% yield) as a yellow solid. ¹H NMR (400 MHz, CDCl₃): δ 8.06 (d, J=8.4 Hz, 1H), 7.52 (bs, 1H), 7.27-7.21 (m, 4H), 6.77 (d, J=8.3 Hz, 2H), 6.01 (tt, J₁=3.8 Hz, J₂=7.9 Hz, 1H), 5.12-4.78 (m, 4H), 3.74 (s, 3H), 3.02 (s, 3H).

### Step 4: Preparation of N-(7-amino-4-chloro-1-(2,2-difluoroethyl)-1H-indazol-3-yl)-N-(4-methoxybenzyl)methanesulfonamide

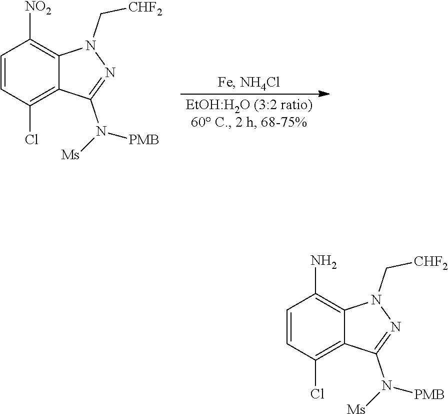

To a stirred suspension of iron powder (76.5 g, 1.37 mol, 5.0 equiv.) in a mixture of EtOH (650 mL, 5.0 V) and water (780 mL, 6.0 V) at room temperature was added ammonium chloride (118.0 g, 2.18 mol, 8.0 equiv.). To the mixture was added N-(4-chloro-1-(2,2-difluoroethyl)-7-nitro-1H-indazol-3-yl)-N-(4-methoxybenzyl)methanesulfonamide (130 g, 0.27 mol, 1.0 equiv.) in EtOH (520 mL, 4.0 V). The reaction mixture was heated to 60° C. and then stirred for 2 h. After completion of the reaction (monitored by in-process TLC/HPLC), the mixture was cooled to room temperature and diluted with ethyl acetate (1.3 L, 10.0 V) and water (390 mL, 3.0 V). The mixture was stirred for 15 min. The mixture was filtered through a pad of Celite and the Celite pad was then extracted with ethyl acetate (650 mL, 5.0 V). The bi-phasic filtrate was partitioned, and the organic phase was reserved while the aqueous layer was extracted with ethyl acetate (650 mL, 5.0 V). The combined organic layers were washed with brine (1.3 L, 10 V), dried over Na₂SO₄, and then concentrated in vacuo to afford a crude solid. To the crude product was added MTBE (650 mL, 5.0 V) and the suspension was stirred for 30 min. at room temperature. The solids were isolated via filtration. Bulk residual water was removed from the solids by maintaining vacuum filtration for 30-45 min. The wet product was dried in a hot air oven (50° C.) for 2 h to afford the title compound N-(7-amino-4-chloro-1-(2,2-difluoroethyl)-1H-indazol-3-yl)-N-(4-methoxy benzyl)methanesulfonamide (100.0 g, 70% yield) as off-white solid. ¹H NMR (400 MHz, CDCl₃): δ 7.21 (d, J=8.5 Hz, 2H), 6.87 (d, J=8.4 Hz, 1H), 6.78 (d, J=8.5 Hz, 2H), 6.52 (d, J=8.3 Hz, 1H), 6.01 (tt, J₁=3.8 Hz, J₂=7.7 Hz, 1H), 4.98-4.69 (m, 4H), 3.75 (s, 3H), 2.98 (s, 3H).

### Preparation of N-(7-amino-4-chloro-1-(2,2-difluoroethyl)-1H-indazol-3-yl)-N-(4-methoxybenzyl)cyclopropanesulfonamide

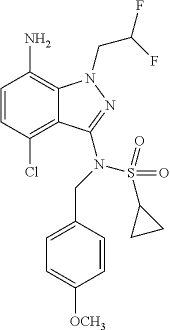 [SMILES: COc1ccc(CN(c2nn(CC(F)F)c3c(N)ccc(Cl)c23)S(=O)(=O)C2CC2)cc1]

### Synthesis Scheme:

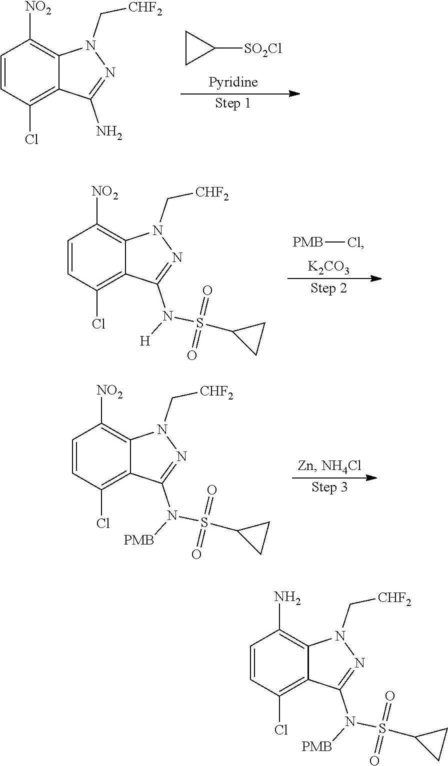

### Step 1: Preparation of N-(4-chloro-1-(2,2-difluoroethyl)-7-nitro-1H-indazol-3-yl)cyclopropanesulfonamide

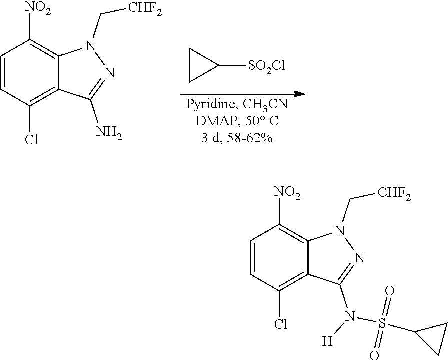 [SMILES: Nc1nn(CC(F)F)c2c([N+](=O)[O-])ccc(Cl)c12 ; O=S(=O)(Cl)C1CC1 ; "Pyridine, CH3CNDMAP, 50° C3 d, 58-62%" ; O=[N+]([O-])c1ccc(Cl)c2c(NS(=O)(=O)C3CC3)nn(CC(F)F)c12]

To a stirred solution of 4-chloro-1-(2,2-difluoroethyl)-7-nitro-1H-indazol-3-amine (150.0 g, 0.54 mol, 1.0 equiv.) in acetonitrile (600 mL, 4.0 V) at room temperature was added pyridine (600 mL, 4.0 V), followed by the addition of 4-dimethylaminopyridine (30.0 g, 0.27 mol, 0.5 equiv.). The reaction mass was stirred for 5-10 min., then cyclopropylsulfonyl chloride (114 mL, 1.08 mol, 2.0 equiv.) was added at room temperature. The reaction mixture was heated to 50° C. and then stirred at that temperature for 3 days. After completion of the reaction (monitored by TLC), the mixture was cooled to room temperature and diluted with water (1.5 L, 10.0 V) and ethyl acetate (1.5 L, 10.0 V), then stirred at room temperature for 15 min. The organic layer was separated, and the aqueous layer was extracted with EtOAc (300 mL, 2.0 V). The combined organic layers were washed with aq. 1.0 N HCl (600 mL, 4.0 V), followed by 10% brine solution (1.5 L, 10.0 V). The organic layer was dried over Na₂SO₄, filtered, and then concentrated under reduced pressure to afford N-(4-chloro-1-(2,2-difluoroethyl)-7-nitro-1H-indazol-3-yl)cyclopropanesulfonamide (124.0 g, 61%) as a viscous liquid. ¹H NMR (400 MHz, CDCl₃): δ 8.11 (d, J=8.5 Hz, 1H), 7.25 (d, J=8.5 Hz, 1H), 6.04 (tt, J₁=3.8 Hz, J₂=7.7 Hz, 1H), 5.05 (td, J₁=3.8 Hz, J₂=14.4 Hz, 2H), 3.06-3.00 (m, 1H), 1.65-1.42 (m, 2H), 1.19-1.13 (m, 2H).

### Step 2: Preparation of N-(4-chloro-1-(2,2-difluoroethyl)-7-nitro-1H-indazol-3-yl)-N-(4-methoxybenzyl)cyclopropanesulfonamide

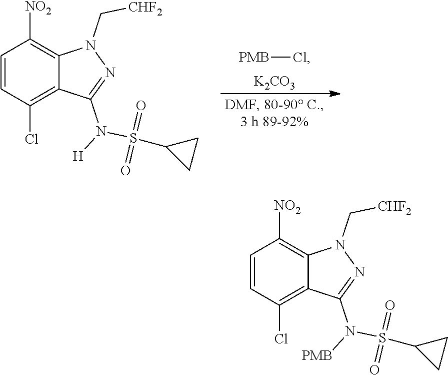

To a mixture of N-(4-chloro-1-(2,2-difluoroethyl)-7-nitro-1H-indazol-3-yl)cyclopropanesulfonamide (100.0 g, 0.20 mol, 1.0 equiv.) and 1-(chloromethyl)-4-methoxybenzene (39.2 mL, 0.22 mol, 1.1 equiv.) in DMF (1.0 L, 10.0 V) at room temperature was added potassium carbonate (128 g, 0.33 mol, 1.3 equiv.). The reaction mixture was heated to 80-90° C. and maintained at that temperature for 3 h. After completion of the reaction (monitored by TLC), the mixture was poured into ice cold water (3.0 L, 30.0 V) [Note: Slow quenching with vigorous stirring is preferred to avoid clumping as the product precipitates]. The resulting solids were isolated via filtration and washed with water (300 mL, 3.0 V); then the solids were washed with hexanes (300 mL, 3.0 V). Bulk residual water was removed from the solids by maintaining vacuum filtration for 1-2 h. The wet solid was dissolved in ethyl acetate (500 mL, 5.0 V) and charcoal was added (10.0 g). The mixture was heated to 60-70° C. and then stirred for 30-45 minutes at that temperature. The mixture was filtered while hot (40-50° C.) through a pad of Celite and the Celite pad was extracted with ethyl acetate (500 mL, 5.0 V). The combined filtrates were concentrated to dryness under reduced pressure at below 50° C. to afford N-(4-chloro-1-(2,2-difluoroethyl)-7-nitro-1H-indazol-3-yl)-N-(4-methoxy-benzyl)cyclopropanesulfonamide (122.0 g, 92% yield) as a yellow solid. ¹H NMR (400 MHz, CDCl₃): δ 8.05 (d, J=8.6 Hz, 1H), 7.26-7.22 (m, 3H), 6.73 (d, J=8.5 Hz, 2H), 5.98 (tt, J₁=3.7 Hz, J₂=7.8 Hz, 1H), 5.09-4.88 (m, 4H), 3.72 (s, 3H), 2.65-2.60 (m, 1H), 1.15-1.06 (m, 2H), 0.89-0.86 (m, 2H).

### Step 3: Preparation of N-(7-amino-4-chloro-1-(2,2-difluoroethyl)-1H-indazol-3-yl)-N-(4-methoxybenzyl)cyclopropanesulfonamide

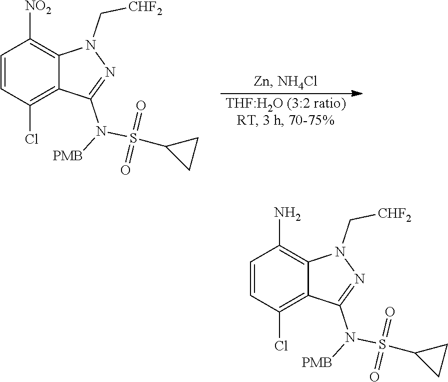

To a stirred suspension of zinc powder (156.0 g, 2.4 mol, 10.0 equiv.) in a mixture of THF (1.2 L, 10.0 V) and water (2.4 L, 20.0 V) at room temperature was added ammonium chloride (129.0 g, 2.40 mol, 10.0 equiv.). To the mixture was added N-(4-chloro-1-(2,2-difluoroethyl)-7-nitro-1H-indazol-3-yl)-N-(4-methoxybenzyl)cyclopropanesulfonamide (120 g, 0.2 mol, 1.0 equiv.) in THF (2.4 L, 20.0 V). The reaction mixture was stirred at room temperature for 2 h. After completion of the reaction (monitored by in-process TLC/HPLC), the mixture was diluted with ethyl acetate (1.2 L, 10.0 V) and water (360 mL, 3.0 V). The mixture was stirred for 15 min. The mixture was filtered through Celite and the Celite pad was extracted with ethyl acetate (600 mL, 5.0 V). The bi-phasic filtrate was partitioned, and the organic phase was reserved while the aqueous layer was extracted with ethyl acetate (600 mL, 5.0 V). The combined organic layers were washed with 10% brine solution (1.2 L, 10 V), dried over Na₂SO₄, filtered, and then concentrated in vacuo to afford a crude solid. To the crude product was added MTBE (600 mL, 5.0 V) and the suspension was stirred for 30-45 min. at room temperature. The solids were isolated by filtration and then bulk residual water was removed from the solids by maintaining vacuum filtration for 30-45 min. The wet product was dried in a hot air oven (50° C.) for 2 h to afford the product, N-(7-amino-4-chloro-1-(2,2-difluoroethyl)-1H-indazol-3-yl)-N-(4-methoxybenzyl)cyclopropanesulfonamide (81.0 g, 73% yield) as off-white solid. ¹H NMR (400 MHz, CDCl₃): δ 7.25 (d, J=8.5 Hz, 2H), 6.93 (d, J=8.4 Hz, 1H), 6.75 (d, J=8.3 Hz, 2H), 6.57 (d, J=8.4 Hz, 1H), 6.03 (tt, J₁=3.7 Hz, J₂=7.9 Hz, 1H), 4.80-4.95 (m, 4H), 3.74 (s, 3H), 2.67-2.61 (m, 1H), 1.14 (d, J=2.4 Hz, 2H), 0.96 (d, J=2.3 Hz, 2H).

### Preparation of N-(7-amino-4-chloro-1-(2,2,2-trifluoroethyl)-1H-indazol-3-yl)-N-(4-methoxybenzyl)methanesulfonamide

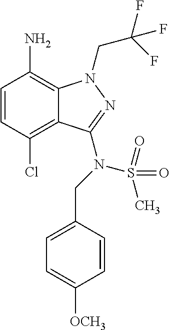 [SMILES: COc1ccc(CN(c2nn(CC(F)(F)F)c3c(N)ccc(Cl)c23)S(C)(=O)=O)cc1]

### Synthesis Scheme:

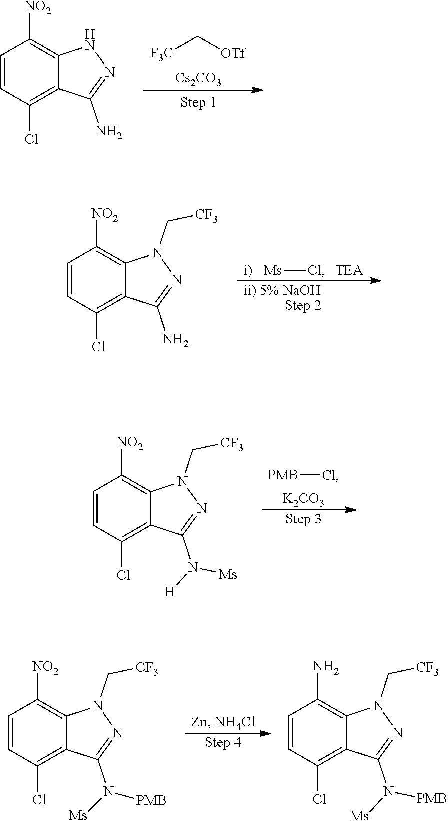

### Step 1: Preparation of 4-chloro-7-nitro-1-(2,2,2-trifluoroethyl)-1H-indazol-3-amine

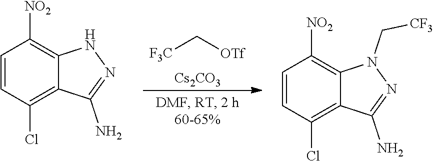

To a stirred solution of 4-chloro-7-nitro-1H-indazol-3-amine (50 g, 0.23 mol, 1.0 equiv.) in DMF (500 mL, 10.0 V) at 10-15° C. was added cesium carbonate (Cs₂CO₃) (153.3 g, 0.47 mol, 2.0 equiv.) at a rate sufficient to maintain the reaction mass below 20° C. The mixture was stirred for 5-10 min, then to the stirred mixture at 10-15° C. was added 2,2,2-trifluoroethyl trifluoromethanesulfonate (60.18 g, 0.26 mol, 1.1 equiv.) at a rate sufficient to maintain the reaction mass below 20° C. (Note: slow addition is preferred for obtaining more favorable regio-selectivity). The reaction mass was allowed to slowly warm to room temperature and was then stirred at the same temperature for 2 h. After completion of the reaction (monitored by TLC), the reaction mass was quenched via the addition of ice-cold water (1.5 L, 30.0 V) and the resulting mixture was allowed to warm to room temperature with stirring for 6-8 h. The solids were isolated via filtration and were then washed with water (150 mL, 3.0 V). The wet solid was washed with hexanes (250 mL, 5.0 V) and then bulk residual water was removed from the solids by maintaining vacuum filtration for 60-90 min. The wet solid was dried in a hot air oven for 7-8 h at 50° C. (until the moisture content was below 1.0%). The isolated material, 4-chloro-7-nitro-1-(2,2,2-trifluoroethyl)-1H-indazol-3-amine (45.0 g, 60% yield), was used directly in the next step without further purification. ¹H-NMR (400 MHz, CDCl₃): δ 8.09 (d, J=8.40 Hz, 1H), 7.12 (d, J=8.40 Hz, 1H), 5.14 (q, J=8.52 Hz, 2H), 4.77 (bs, H).

### Step 2: Preparation of N-(4-chloro-7-nitro-1-(2,2,2-trifluoroethyl)-1H-indazol-3-yl)methanesulfonamide

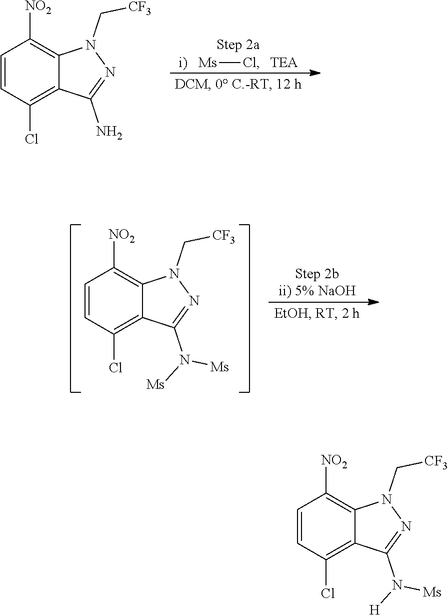

(Step 2a): To a solution of 4-chloro-7-nitro-1-(2,2,2-trifluoroethyl)-1H-indazol-3-amine (20.0 g, 0.068 mol, 1.0 equiv.) in DCM (200 mL, 10.0 V) at 0-5° C. was added triethylamine (29.0 mL, 0.204 mol, 3.0 equiv.), followed by the addition of 4-dimethylaminopyridine (415 mg, 0.03 mol, 0.05 equiv.). The reaction mass was stirred for 5-10 min., then to the mixture was added methanesulfonyl chloride (13.25 mL, 0.17 mol, 2.5 equiv) at a rate sufficient to maintain the reaction mass below 10° C. The reaction mixture was allowed to warm to room temperature with stirring for 12 h. After completion of the reaction (monitored by TLC), the mixture was diluted with water (200 mL, 10.0 V) and then stirred at room temperature for 15 min. The organic layer was separated, and the aqueous layer was extracted with DCM (200 mL, 10.0 V). The combined organic layers were washed with 10% brine solution (60 mL, 3.0 V), dried over Na₂SO₄, filtered, and concentrated to afford the crude solids. The solids were triturated with hexanes (60 mL, 3.0 V) at room temperature to obtain the intermediate, N-(4-chloro-7-nitro-1-(2,2,2-trifluoroethyl)-1H-indazol-3-yl)-N-(methylsulfonyl)methanesulfonamide, which was used directly in the next step.
(Step 2b): To a stirred solution of N-(4-chloro-7-nitro-1-(2,2,2-trifluoroethyl)-1H-indazol-3-yl)-N-(methylsulfonyl)methanesulfonamide (entirety of the material prepared above) in ethanol (200 mL, 10.0 V) at room temperature was added slowly aq. 5% NaOH solution (140 mL, 7.0 V) [Note: Slow addition is preferred via dropping funnel]. The reaction mass was stirred at the same temperature for 2 h. After completion of the reaction [Sample preparation for TLC analysis: An aliquot of the reaction solution (˜1.0 ml) was acidified by the addition of aq. 2.0 N HCl to reach pH 2-3; then the mixture was extracted with ethyl acetate and the organic phase was analyzed by TLC], the reaction mass was cooled to 0-5° C. and the pH was adjusted to 2-3 by the addition of aq. 2.0 N HCl (100 mL, 5.0 V) while maintain the temperature below 10° C. [Note: Precipitation occurred upon addition of HCl and increased with stirring]. The reaction mixture was warmed to room temperature and then stirred for 1.5-2.0 h. The solids were isolated via filtration and were then washed with water (60 mL, 3.0 V), followed by washing with hexanes (60 mL, 3.0 V). Bulk residual water was removed from the solids by maintaining vacuum filtration for 60-90 min. The wet material was dried in a hot air oven at 50° C. for 6-7 h (until the moisture content was below 1.0%) to afford N-(4-chloro-7-nitro-1-(2,2,2-trifluoroethyl)-1H-indazol-3-yl)methanesulfonamide (22.1 g, 87%) as a yellow solid. ¹H NMR (400 MHz, CDCl₃): δ 8.19 (d, J=8.40 Hz, 1H), 7.56 (bs, 1H), 7.30 (d, J=8.40 Hz, 1H), 5.34 (q, J=8.30 Hz, 2H), 3.46 (s, 3H).

### Step 3: Preparation of N-(4-chloro-7-nitro-1-(2,2,2-trifluoroethyl)-1H-indazol-3-yl)-N-(4-methoxybenzyl)methanesulfonamide

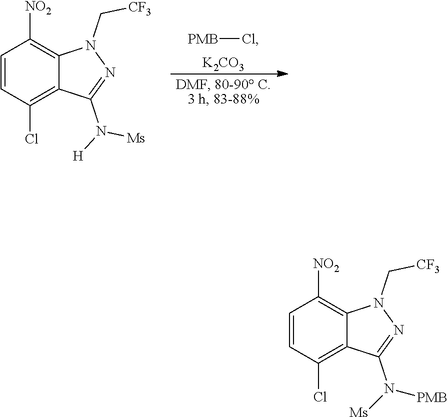

To a mixture of N-(4-chloro-7-nitro-1-(2,2,2-trifluoroethyl)-1H-indazol-3-yl)methanesulfonamide (50.0 g, 0.134 mol, 1.0 equiv.) and 1-(chloromethyl)-4-methoxybenzene (23.0 g, 0.147 mol, 1.1 equiv.) in DMF (500 mL, 10.0 V) at room temperature was added potassium carbonate (27.8 g, 0.201 mol, 1.5 equiv.). The reaction mixture was heated to 80-90° C. and maintained at that temperature for 3 h. After completion of the reaction (monitored by TLC), the mixture was poured into ice cold water (2.0 L, 40.0 V) [Note: Slow quenching with vigorous stirring is preferred to avoid clumping as the product precipitates]. The resulting solids were isolated via filtration and washed with water (150 mL, 3.0 V); then the solids were washed with hexanes (150 mL, 3.0 V). Bulk residual water was removed from the solids by maintaining vacuum filtration for 1-2 h. The solids were dissolved in ethyl acetate (500 mL, 10.0 V) and to the solution was added charcoal (5.0 g). The mixture was heated to 60-70° C. and then stirred at that temperature for 30-45 min. The mixture was filtered while hot (40-50° C.) through a pad of Celite and the Celite pad was extracted with ethyl acetate (250 mL, 5.0 V). The combined filtrate was concentrated to dryness under reduced pressure at below 50° C. The solids were combined with ethyl acetate (50 mL, 1.0 V) at room temperature. The resulting suspension was stirred for 30 min. The solids were isolated via filtration and then were washed with hexanes (100 mL, 2.0 V). Residual water was removed from the solids by maintaining vacuum filtration for 45-60 min. to afford N-(4-chloro-7-nitro-1-(2,2,2-trifluoroethyl)-1H-indazol-3-yl)-N-(4-methoxybenzyl)methanesulfonamide (56.0 g, 85% yield) as a yellow solid. ¹H NMR (400 MHz, CDCl₃): δ 8.12 (d, J=8.36 Hz, 1H), 7.31 (d, J=8.36 Hz, 1H), 7.22 (d, J=8.44 Hz, 2H), 6.77 (d, J=8.44 Hz, 2H), 5.50-5.25 (m, 2H), 4.94-4.79 (m, 2H), 3.75 (s, 3H), 3.02 (s, 3H).

### Step 4: Preparation of N-(7-amino-4-chloro-1-(2,2,2-trifluoroethyl)-1H-indazol-3-yl)-N-(4-methoxybenzyl)methanesulfonamide

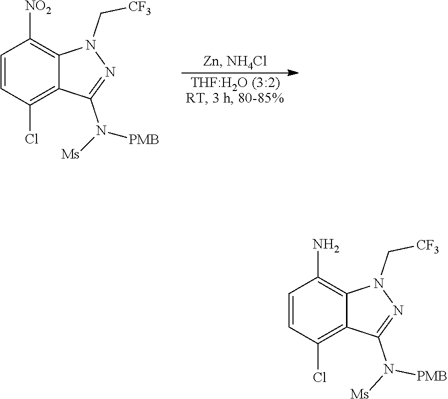

To a stirred suspension of zinc powder (66.31 g, 1.01 mol, 10.0 equiv.) in THF (500 mL, 10.0 V) and water (1.0 L, 20.0 V) at room temperature was added ammonium chloride (54.78 g, 1.01 mol, 10.0 equiv.). To the mixture was added a solution of N-(4-chloro-7-nitro-1-(2,2,2-trifluoroethyl)-1H-indazol-3-yl)-N-(4-methoxybenzyl)methanesulfonamide (50.0 g, 0.101 mol, 1.0 equiv.) in THF (1.0 L, 20.0 V). The reaction mixture was stirred at room temperature for 3 h. After completion of the reaction (monitored by in-process TLC/HPLC), the mixture was diluted with ethyl acetate (1.0 L, 20.0 V) and water (250 mL, 5.0 V). The mixture was stirred for 15 min. The mixture was filtered through a pad of Celite and the Celite pad was extracted with ethyl acetate (250 mL, 5.0 V). The bi-phasic filtrate was partition and the organic layer was reserved while the aqueous layer was extracted with ethyl acetate (500 mL, 10.0 V). The combined organic layers were washed with 10% brine solution (500 mL, 10.0 V), dried over Na₂SO₄, filtered, and then concentrated in vacuo to afford a crude solid. To the crude product was added MTBE (250 mL, 5.0 V) and the resulting suspension was stirred for 30 min. at room temperature. The solids were isolated by filtration and then bulk residual water was removed from the solids by maintaining vacuum filtration for 30-45 min. The wet product was dried in a hot air oven (50° C.) for 2 h to afford the title product N-(7-amino-4-chloro-1-(2,2,2-trifluoroethyl)-1H-indazol-3-yl)-N-(4-methoxybenzyl)methanesulfonamide (39.0 g, 83% yield) as off-white solid. ¹H NMR (400 MHz, CDCl₃): δ 7.25 (d, J=8.48 Hz, 2H), 6.98 (d, J=7.80 Hz, 1H), 6.79 (d, J=8.48 Hz, 2H), 6.66 (d, J=7.84 Hz, 1H), 5.35-4.75 (m, 4H), 3.77 (s, 3H), 3.56 (bs, 2H), 2.98 (s, 3H).

### Preparation of (S)-tert-butyl (1-(3-(4-chloro-1-(2,2-difluoroethyl)-3-(N-(4-methoxybenzyl)methylsulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)carbamate

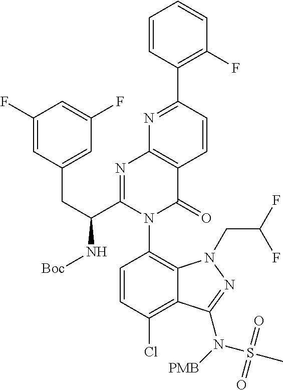

A mixture of 2-amino-6-(2-fluorophenyl)nicotinic acid (1.5 g, 6.46 mmol), (S)-2-((tert-butoxycarbonyl)amino)-3-(3,5-difluorophenyl)propanoic acid (1.752 g, 5.81 mmol), N-(7-amino-4-chloro-1-(2,2-difluoroethyl)-1H-indazol-3-yl)-N-(4-methoxybenzyl)methanesulfonamide (2.59 g, 5.81 mmol) and diphenyl phosphonate (4.95 mL, 25.8 mmol) in pyridine (13 mL) in a pressure vial was stirred for 6 hours at 75° C. The mixture was cooled to rt and the mixture was diluted with EtOAc (˜250 mL). The solution was washed with 0.5 M citric acid, dried over Na₂SO₄, filtered, and concentrated under reduced pressure. The resulting residue was purified by silica gel chromatography (330 g RediSep column) eluting with 0-45% ethyl acetate in hexanes over 12 CV. Fractions containing the desired product were pooled and then concentrated under reduced pressure to afford (S)-tert-butyl (1-(3-(4-chloro-1-(2,2-difluoroethyl)-3-(N-(4-methoxybenzyl)methylsulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)carbamate as a pale yellow foamy solid, 1.61 g (27% yield), a mixture of four stereoisomers. LC/MS: m/z=946.15 [M+1]⁺.

### Preparation of (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-(2,2-difluoroethyl)-1H-indazol-3-yl)-N-(4-methoxybenzyl)methanesulfonamide

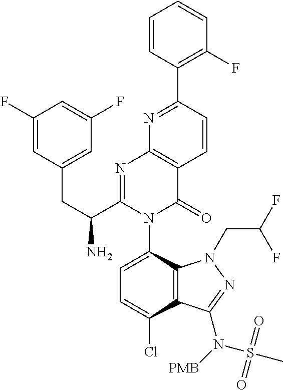

To a solution of tert-butyl (S)-(1-(3-(4-chloro-1-(2,2-difluoroethyl)-3-(N-(4-methoxybenzyl)methylsulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)carbamate (1.93 g, 2.088 mmol) in dichloromethane (10.44 mL) was added HCl in dioxane (4M, 10.44 ml, 41.8 mmol). The mixture was stirred at rt for 1 h. The resulting pale yellow solution was concentrated under reduced pressure and the residue was dissolved in ethyl acetate. The solution was washed with sat. aq. NaHCO₃, dried over Na₂SO₄, filtered, and concentrated under reduced pressure to afford a yellow solid. This material was purified by silica gel chromatography (220 g RediSep column) eluting with 10-100% ethyl acetate in hexanes over 25 column volumes. Two peaks contained the desired product mass; fractions of the major peak (first to elute) were pooled and then concentrated to afford a pale yellow solid. The material was subjected to prep-SFC chromatography (to separate enantiomers) using the following method: Column=Chiralpak IB 20×250 mm, 5 u; Solvent=CO₂:EtOH (65:35); Flow rate=50 g/min; Back pressure=100 bar; UV wavelength=220 nm; Temperature=30° C. The major peak was collected and concentrated to afford (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-(2,2-difluoroethyl)-1H-indazol-3-yl)-N-(4-methoxybenzyl)methanesulfonamide, 1.2 g, (69% yield, 100% chiral purity). LC/MS: m/z=824.10 [M+1]⁺.

### Preparation of (S)-tert-butyl (1-(3-(4-chloro-1-(2,2-difluoroethyl)-3-(N-(4-methoxybenzyl)cyclopropanesulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)carbamate

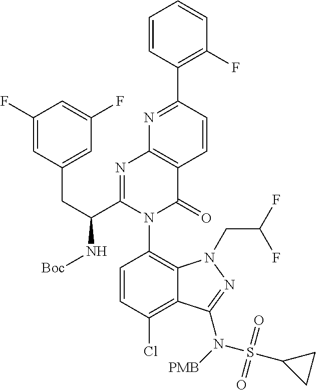

A mixture of 2-amino-6-(2-fluorophenyl)nicotinic acid 0.493 g, 2.124 mmol), (S)-2-((tert-butoxycarbonyl)amino)-3-(3,5-difluorophenyl)propanoic acid (0.640 g, 2.124 mmol), N-(7-amino-4-chloro-1-(2,2-difluoroethyl)-1H-indazol-3-yl)-N-(4-methoxybenzyl)cyclopropanesulfonamide (1 g, 2.124 mmol) and diphenyl phosphonate (1.627 ml, 8.49 mmol) in pyridine (4.25 mL) in a pressure vial was stirred at 75° C. for 8 h. The mixture was cooled to rt and then was diluted with EtOAc, washed with 0.5 M citric acid, dried over Na₂SO₄, filtered, and concentrated under reduced pressure. The resulting residue was purified by silica gel chromography (220 g RediSep column) eluting with 0-40% ethyl acetate in hexanes over 25 CV. The desired fractions were pooled and concentrated under reduced pressure to afford (S)-tert-butyl (1-(3-(4-chloro-1-(2,2-difluoroethyl)-3-(N-(4-methoxybenzyl)cyclopropanesulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)carbamate as a pale yellow foamy solid, 0.97 g (48%), a mixture of four stereoisomers. LC/MS: m/z=950.10 [M+1]⁺.

### Preparation of (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-(2,2-difluoroethyl)-1H-indazol-3-yl)-N-(4-methoxybenzyl)cyclopropanesulfonamide

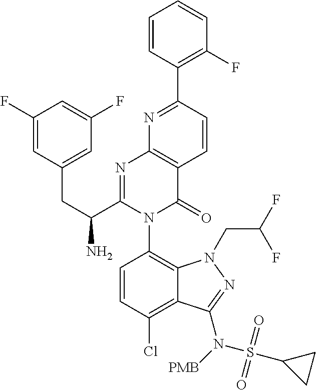

To a solution of tert-butyl (S)-(1-(3-(4-chloro-1-(2,2-difluoroethyl)-3-(N-(4-methoxybenzyl)cyclopropanesulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)carbamate (0.971 g, 1.022 mmol) in dichloromethane (5 mL) was added HCl in dioxane (4M, 5.11 mL, 20.43 mmol). The mixture was stirred at rt for 1 h. The resultant pale yellow solution was concentrated under reduced pressure. The residue was dissolved in ethyl acetate and then was washed with sat. aq. NaHCO₃, dried over Na₂SO₄, filtered, and concentrated in vacuo afford a yellow solid. This material was purified by silica gel chromatography (220 g RediSep column) eluting with 10-100% ethyl acetate in hexanes over 25 column volumes. Two peaks contained the desired product mass; fractions of the major peak (first to elute) were pooled and then concentrated to afford a pale yellow solid. This material was subjected to prep-HPLC (chiral) chromatography (to separate enantiomers) using the following method: Column=Chiralcel OD-H (30 mm×250 mm), 5 microns; Eluent=n-heptane:ethanol (40:60) with 20 mM ammonium acetate; Flow rate=45 ml/min.; Temperature=ambient temp.; Detection=260 nm (UV). The major peak was collected and concentrated in vacuo to afford (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-(2,2-difluoroethyl)-1H-indazol-3-yl)-N-(4-methoxybenzyl)cyclopropanesulfonamide, 0.51 g (59% yield, 100% chiral purity). LC/MS: m/z=850.10 [M+1]⁺.

### Preparation of (S)-tert-butyl (1-(3-(4-chloro-3-(N-(4-methoxybenzyl)methylsulfonamido)-1-(2,2,2-trifluoroethyl)-1H-indazol-7-yl)-7-(2-fluorophenyl)-4-oxo-3,4-dihydropyrido[2,3-d]pyrimidin-2-yl)-2-(3,5-difluorophenyl)ethyl)carbamate

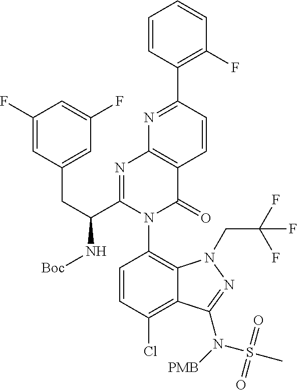

A mixture of 2-amino-6-(2-fluorophenyl)nicotinic acid (1 g, 4.31 mmol), (S)-2-((tert-butoxycarbonyl)amino)-3-(3,5-difluorophenyl)propanoic acid (1.297 g, 4.31 mmol), N-(7-amino-4-chloro-1-(2,2,2-trifluoroethyl)-1H-indazol-3-yl)-N-(4-methoxybenzyl)methanesulfonamide (1.993 g, 4.31 mmol) and diphenyl phosphonate (3.30 mL, 17.23 mmol) in pyridine (12.30 mL) in a pressure vial was stirred at 75° C. for 18 h. The mixture was cooled to rt and then was diluted with EtOAc and washed with aq. 0.5 M citric acid, dried over Na₂SO₄, filtered, and then concentrated under reduced pressure. The resulting residue was purified by silica gel chromatography (80 g RediSep column) eluting with 0-45% ethyl acetate in hexanes over 25 CV. The desired fractions were pooled and then concentrated in vacuo to afford tert-butyl (S)-(1-(3-(4-chloro-3-(N-(4-methoxybenzyl)methylsulfonamido)-1-(2,2,2-trifluoroethyl)-1H-indazol-7-yl)-7-(2-fluorophenyl)-4-oxo-3,4-dihydropyrido[2,3-d]pyrimidin-2-yl)-2-(3,5-difluorophenyl)ethyl)carbamate as a pale yellow foamy solid, 1.44 g (36%), a mixture of four stereoisomers. LC/MS: m/z=942.10 [M+1]⁺.

### Preparation of (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-(2,2,2-trifluoroethyl)-1H-indazol-3-yl)methanesulfonamide

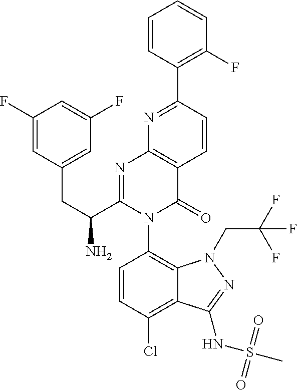 [SMILES: CS(=O)(=O)Nc1nn(CC(F)(F)F)c2c(-n3c([C@@H](N)Cc4cc(F)cc(F)c4)nc4nc(-c5ccccc5F)ccc4c3=O)ccc(Cl)c12]

To a solution of tert-butyl (S)-(1-(3-(4-chloro-3-(N-(4-methoxybenzyl)methylsulfonamido)-1-(2,2,2-trifluoroethyl)-1H-indazol-7-yl)-7-(2-fluorophenyl)-4-oxo-3,4-dihydropyrido[2,3-d]pyrimidin-2-yl)-2-(3,5-difluorophenyl)ethyl)carbamate (1.44 g, 1.528 mmol) in dichloromethane (5.0 ml) and trifluoroacetic acid (2.5 mL) was added trifluoromethanesulfonic acid (0.407 mL, 4.58 mmol). The solution was stirred at rt for 1 h and then the pale yellow solution was concentrated in vacuo. The resulting residue was dissolved in ethyl acetate, washed with aq. 1 N NaOH, dried over Na₂SO₄, filtered, and concentrated under reduced pressure to afford a yellow solid. The solid was purified by silica gel chromatography (120 g RediSep Gold column) eluting with 10-100% ethyl acetate in hexanes over 20 CV, then at 100% ethyl acetate for 5 CV and finally at 12.5% methanol in ethyl acetate for 3 CV. Two peaks contained the desired product mass; fractions of the major peak (first to elute) were pooled and concentrated under reduced pressure to afford a white fluffy solid. This material was further purified by prep-HPLC (chiral, to separate enantiomers) using the following method: Column=Chiralpak IG 20×250 mm, 5 micron; Eluent=heptane:ethanol (60:40), no modified was used during elution but the column was pre-conditioned with 100 mM NH₄OAc in ethanol; flow-rate=20 mL/min.; Detection=254 nm (UV); length of run=10 min. Two peaks were observed with retention times of 6.5 min and 8.6 min. Fractions corresponding to the major peak were pooled and concentrated to afford (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-(2,2,2-trifluoroethyl)-1H-indazol-3-yl)methanesulfonamide, 0.41 g (37% yield, 100% chiral purity). LC/MS: m/z=722 [M+1]⁺.

### Preparation of methyl 2-amino-6-(2,4-difluorophenyl)nicotinate

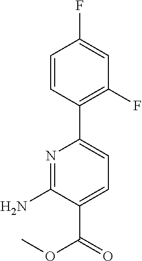 [SMILES: COC(=O)c1ccc(-c2ccc(F)cc2F)nc1N]

A mixture of methyl 2-amino-6-chloronicotinate (2 g, 10.72 mmol), (2,4-difluorophenyl)boronic acid (2.370 g, 15.01 mmol), PdCl2(dppf) (0.392 g, 0.536 mmol) and Na₂CO₃ (2.272 g, 21.44 mmol) in 1,4-dioxane (32.0 mL) and water (8.00 mL) was degassed (the flask is evacuated and then refilled with nitrogen; the process is repeated three times and then N2 atmosphere is maintained) at room temperature and then the mixture was stirred at 100° C. for 8 h. The mixture was cooled to rt, diluted with ethyl acetate, filtered through Celite to remove solids, and the filtrate was partitioned. The organic layer was dried over Na₂SO₄, filtered, and then concentrated under reduced pressure. The resulting residue was purified by silica gel chromatography (220 g RediSep column) eluting with 0-40% ethyl acetate in hexanes over 20 CV, then at 40% ethyl acetate for 10 CV. The desired fractions were pooled and then concentrated under reduced pressure to afford methyl 2-amino-6-(2,4-difluorophenyl)nicotinate (1.31 g, 46% yield) as a pale yellow solid. ¹H NMR (500 MHz, CHLOROFORM-d) δ ppm 7.98-8.06 (m, 1H) 7.81-7.90 (m, 1H) 6.93-6.98 (m, 1H) 6.79-6.87 (m, 1H) 6.68-6.77 (m, 1H) 3.70-3.77 (m, 3H). LC/MS: m/z=265.05 [M+1]⁺.

### Preparation of 2-amino-6-(2,4-difluorophenyl)nicotinic acid

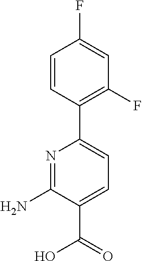 [SMILES: Nc1nc(-c2ccc(F)cc2F)ccc1C(=O)O]

To a solution of methyl 2-amino-6-(2,4-difluorophenyl)nicotinate (1.31 g, 4.96 mmol) in THF:MeOH (6:2, 24 mL) at room temperature was slowly added a solution of sodium hydroxide (0.793 g, 19.83 mmol) in water (3 mL) (an exotherm was noted). The mixture was stirred at rt for 6 h, then the suspension was stirred at 60° C. for 16 h. To the suspension was added solid NaOH pellets (1.6 g, 39.65 mmol.) and then the mixture was stirred at 60° C. for 4 h upon which LMCS analysis indicated complete conversion. The reaction mixture (a light pink suspension) was concentrated under reduced pressure to remove the volatile organics. The resulting aqueous mixture was diluted with water and then acidified to pH ˜2 by the addition of aq. 1N HCl at 0° C. The resulting precipitate was collected by filtrated and bulk water was removed by maintaining the solids under vacuum filtrate. Once bulk water was removed, the solid was furthered dried in a vacuum oven at 40° C. overnight to afford 2-amino-6-(2,4-difluorophenyl)nicotinic acid (1.36 g) as a pale yellow solid. ¹H NMR (500 MHz, DMSO-d₆) δ ppm 8.05-8.13 (m, 1H) 7.93-8.02 (m, 1H) 7.32-7.39 (m, 1H) 7.18-7.24 (m, 1H) 6.90-6.96 (m, 1H) 3.23-3.48 (m, 2H). LC/MS: m/z=251.05 [M+1]⁺.

### Preparation of (S)-tert-butyl (1-(3-(4-chloro-3-(N-(4-methoxybenzyl)methylsulfonamido)-1-methyl-1H-indazol-7-yl)-7-(2,4-difluorophenyl)-4-oxo-3,4-dihydropyrido[2,3-d]pyrimidin-2-yl)-2-(3,5-difluorophenyl)ethyl)carbamate

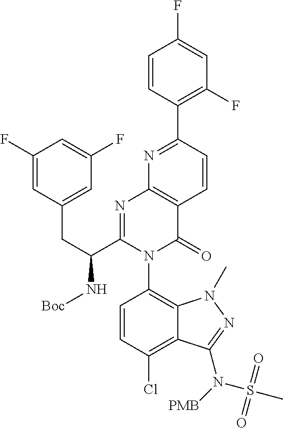

A mixture of 2-amino-6-(2,4-difluorophenyl)nicotinic acid (0.950 g, 3.80 mmol), (S)-2-((tert-butoxycarbonyl)amino)-3-(3,5-difluorophenyl)propanoic acid (1.145 g, 3.80 mmol), N-(7-amino-4-chloro-1-methyl-1H-indazol-3-yl)-N-(4-methoxybenzyl)methanesulfonamide (1.5 g, 3.80 mmol) and diphenyl phosphonate (2.90 mL, 15.19 mmol) in pyridine (7.60 mL) in a pressure vial was stirred at 75° C. for 8 h. The solution was cooled to rt and was diluted with EtOAc, washed with aq. 0.5 M citric acid, dried over Na₂SO₄, filtered, and then concentrated under reduced pressure. The resulting residue was purified by silica gel chromatography (330 g RediSep column) eluting with 0-40% ethyl acetate in hexanes over 25 CV. The desired fractions were pooled and then concentrated in vacuo to afford tert-butyl (S)-(1-(3-(4-chloro-3-(N-(4-methoxybenzyl)methylsulfonamido)-1-methyl-1H-indazol-7-yl)-7-(2,4-difluorophenyl)-4-oxo-3,4-dihydropyrido[2,3-d]pyrimidin-2-yl)-2-(3,5-difluorophenyl)ethyl)carbamate (1.2 g, 35%) as a pale yellow solid foam, a mixture of four stereoisomers. LC/MS: m/z=914.15 [M+Na]⁺.

### Preparation of (S)—N-((6P)-7-(2-(1-amino-2-(3,5-difluorophenyl)ethyl)-7-(2,4-difluorophenyl)-4-oxopyrido[2,3-d]pyrimidin-3(4H)-yl)-4-chloro-1-methyl-1H-indazol-3-yl)-N-(4-methoxybenzyl)methanesulfonamide

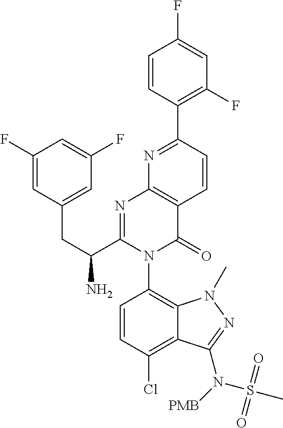

To a solution of tert-butyl (S)-(1-(3-(4-chloro-3-(N-(4-methoxybenzyl)methylsulfonamido)-1-methyl-1H-indazol-7-yl)-7-(2,4-difluorophenyl)-4-oxo-3,4-dihydropyrido[2,3-d]pyrimidin-2-yl)-2-(3,5-difluorophenyl)ethyl)carbamate (1.2 g, 1.345 mmol) in dichloromethane (9 mL) was added HCl in dioxane (4M, 6.7 mL, 26.9 mmol). The solution was stirred at rt for 1 h, then the pale yellow solution was concentrated in vacuo. The resulting residue was dissolved in ethyl acetate, washed with aq. 1 N NaOH, dried over Na₂SO₄, filtered, and then concentrated under reduced pressure to afford a yellow solid. The solid was purified by silica gel chromatography (2×80 g RediSep Gold columns connected in series) eluting with 10-100% ethyl acetate in hexanes over 12 CV, then at 100% ethyl acetate for 10 CV and finally at 12.5% methanol in ethyl acetate for 3 CV. Two peaks contained the desired product mass; fractions of the major peak (first to elute) were pooled and then concentrated to afford a pale yellow solid. This material was purified by prep-SFC (to separate enantiomers) using the following method: Column=ChiralCel OD-H 21×250 mm; Eluent=CO₂:Methanol (70:30) w/0.25% diethylamine; Flow rate=70 mL/min.; Sample solubility=60 mg/mL in methanol:dichloromethane (1:1); Injection=1.5 mL; Detection=254 nm (UV). This separation afforded two peaks; fraction of the major peak were pooled and concentrated to afford (S)—N-((6P)-7-(2-(1-amino-2-(3,5-difluorophenyl)ethyl)-7-(2,4-difluorophenyl)-4-oxopyrido[2,3-d]pyrimidin-3(4H)-yl)-4-chloro-1-methyl-1H-indazol-3-yl)-N-(4-methoxybenzyl)methanesulfonamide (0.59 g, 55% yield, 98.2% chiral purity) LC/MS: m/z=792.10 [M+1]+.

### Preparation of (S)-tert-butyl (1-(7-bromo-3-(4-chloro-3-(N-(4-methoxybenzyl)methylsulfonamido)-1-methyl-1H-indazol-7-yl)-4-oxo-3,4-dihydropyrido[3,2-d]pyrimidin-2-yl)-2-(3,5-difluorophenyl)ethyl)carbamate

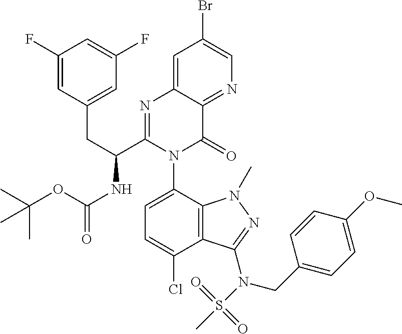 [SMILES: COc1ccc(CN(c2nn(C)c3c(-n4c([C@H](Cc5cc(F)cc(F)c5)NC(=O)OC(C)(C)C)nc5cc(Br)cnc5c4=O)ccc(Cl)c23)S(C)(=O)=O)cc1]

To a suspension of (S)-2-((tert-butoxycarbonyl)amino)-3-(3,5-difluorophenyl)propanoic acid (6.41 g, 21.27 mmol) and 3-amino-5-bromopicolinic acid (4.62 g, 21.27 mmol) in acetonitrile (107 mL) at −25° C. was added pyridine (11.5 mL, 142 mmol) followed by 2,4,6-tripropyl-1,3,5,2,4,6-trioxatriphosphinane 2,4,6-trioxide (“T3P”, 52.8 mL, 89 mmol) upon which the suspension became a clear solution. The reaction solution was stirred while the temperature slowly rose from −25° C. to 12 C over 5 h. To the solution was added N-(7-amino-4-chloro-1-methyl-1H-indazol-3-yl)-N-(4-methoxybenzyl)methanesulfonamide (7 g, 17.73 mmol). The mixture was allowed to warm to room temperature with stirring and was then stirred for 3 days at room temperature. The reaction mixture was warmed to 40° C. upon which the mixture became a homogeneous solution. The solution was allowed to cool to room temperature and was then stirred for 18 h. The reaction mixture was diluted with ethyl acetate, washed with aq. 1N NaOH, then water, then aq. 0.5 M citric acid, and then water. The solution was dried over Na₂SO₄, filtered, and then concentrated under reduced pressure. The resulting residue was purified by silica gel chromatography (330 g RediSep Gold column) eluting with 0-60% ethyl acetate in hexanes over 15 CV, then at 60% ethyl acetate in hexanes for 10 CV. The desired fractions were pooled and concentrated under reduced pressure to afford(S)-tert-butyl (1-(7-bromo-3-(4-chloro-3-(N-(4-methoxybenzyl)methylsulfonamido)-1-methyl-1H-indazol-7-yl)-4-oxo-3,4-dihydropyrido[3,2-d]pyrimidin-2-yl)-2-(3,5-difluorophenyl)ethyl)carbamate as a pale yellow solid, 8.25 g (54% yield), a mixture of four stereoisomers. LC/MS: m/z=858.10 [M+1]+.

### Preparation of (S)—N-((6P)-7-(2-(1-amino-2-(3,5-difluorophenyl)ethyl)-7-bromo-4-oxopyrido[3,2-d]pyrimidin-3(4H)-yl)-4-chloro-1-methyl-1H-indazol-3-yl)methanesulfonamide

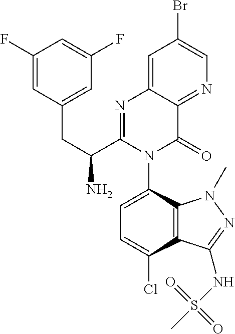

To a solution of tert-butyl-(1-(7-bromo-3-(4-choro-3-N-(4-methoxybenzyl)methylsulfonamido)-1-methyl-1H-indazol-7-yl)-4-oxo-3,4-dihydropyrido[3,2-d]pyrimidin-2-yl)-2-(3,5-difluorophenyl)ethyl)carbamate (8.25 g, 9.60 mmol) in dichloromethane (32.0 mL) was added TFA (14.80 mL, 192 mmol) and triflic acid (2.56 mL, 28.8 mmol). The solution was stirred at rt for 2 h. The pale yellow solution was concentrated under reduced pressure and the residue was dissolved in ethyl acetate. The solution was washed with aq. 1 N NaOH and then aq. 0.5 M citric acid, dried over Na₂SO₄, and filtered. The filtrate was adsorbed onto Celite in vacuo and the resulting powder was subjected to silica gel chromatography (2×330 g RediSep Gold columns connected in series) using a gradient method of hexanes:(hexanes:EtOAc:MeOH, 9:9:2) 75:25→0:100 over 2 CV, then 100% of 9:9:2 of (hexanes:EtOAc:MeOH, 9:9:2) 20 CV. This chromatography afforded two peaks containing the desired product mass; fraction corresponding to the major peak (first to elute) were pooled and then concentrated in vacuo to afford (S)—N-((6P)-7-(2-(1-amino-2-(3,5-difluorophenyl)ethyl)-7-bromo-4-oxopyrido[3,2-d]pyrimidin-3(4H)-yl)-4-chloro-1-methyl-1H-indazol-3-yl)methanesulfonamide as a colorless solid foam, 3.8 g (62% yield), a single stereoisomer. LC/MS: m/z=638 [M+1]+.

### Preparation of N—((S)-1-((3P)-7-bromo-3-(4-chloro-1-methyl-3-(methylsulfonamido)-1H-indazol-7-yl)-4-oxo-3,4-dihydropyrido[3,2-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

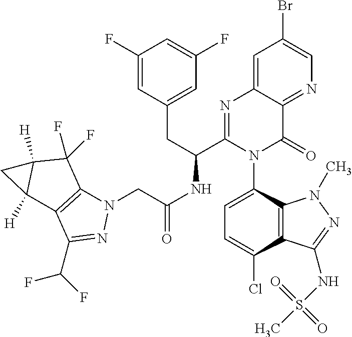

To a stirred solution of (S)—N-((6P)-7-(2-(1-amino-2-(3,5-difluorophenyl)ethyl)-7-bromo-4-oxopyrido[3,2-d]pyrimidin-3(4H)-yl)-4-chloro-1-methyl-1H-indazol-3-yl)methanesulfonamide (0.05 g, 0.078 mmol) in tetrahydrofuran (1 mL) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.021 g, 0.078 mmol), 2,4,6-tripropyl-1,3,5,2,4,6-trioxatriphosphinane 2,4,6-trioxide (“T3P”, 0.093 mL, 0.157 mmol) and DIPEA (0.041 mL, 0.235 mmol). The reaction mixture was stirred for 3 h at rt, then the mixture was directly subjected to silica gel chromatography (24 g RediSep Gold column) eluting with 10-80% ethyl acetate in hexanes over 15 CV, then at 80% ethyl acetate in hexanes for 10 CV. The desired fractions were pooled and then concentrated in vacuo to afford N—((S)-1-((3P)-7-bromo-3-(4-chloro-1-methyl-3-(methylsulfonamido)-1H-indazol-7-yl)-4-oxo-3,4-dihydropyrido[3,2-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (0.044 g, 64%) as a pale yellow solid. ¹H NMR (500 MHz, METHANOL-d₄) δ ppm 8.93-8.98 (m, 1H) 8.51-8.57 (m, 1H) 7.27-7.35 (m, 2H) 6.56-6.82 (m, 4H) 4.80 (dd, J=9.54, 4.47 Hz, 1H) 4.44-4.53 (m, 2H) 3.60-3.64 (m, 3H) 3.41-3.47 (m, 1H) 3.23-3.25 (m, 3H) 3.05-3.11 (m, 1H) 2.37-2.45 (m, 2H) 1.31-1.37 (m, 1H) 0.94-0.99 (m, 1H). LC/MS: m/z=885.75[M+2]+.

### Preparation of 2-amino-6-(benzyloxy)nicotinic acid

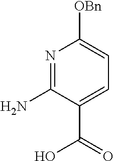 [SMILES: Nc1nc(OCc2ccccc2)ccc1C(=O)O]

A solution of 2-amino-6-chloronicotinic acid (5 g, 29 mmol) and potassium tert-butoxide (9.75 g, 87 mmol) in benzyl alcohol (97 mL) was stirred at 120° C. for 3 h. After cooling to ambient temperature, the very dark reaction mixture was diluted with water and then washed with ether (×3). The aqueous layer was then acidified with 0.5 M citric acid. The tan precipitate was isolated by filtration to provide the product (4.4 g, 62%) which was used in the next reaction without further purification. ¹H NMR (500 MHz, DMSO-d6) δ 12.40 (br s, 1H), 7.94 (d, J=8.55 Hz, 1H), 7.06-7.52 (m, 5H), 6.04 (d, J=8.24 Hz, 1H), 5.33 (s, 2H). LC/MS: m/z=245.15 [M+1]⁺.

### Preparation of N-[(6P)-7-{2-[(1S)-1-amino-2-(3,5-difluorophenyl)ethyl]-7-hydroxy-4-oxo-3H,4H-pyrido[2,3-d]pyrimidin-3-yl}-4-chloro-1-methyl-1H-indazol-3-yl]-N-[(4-methoxyphenyl)methyl]methanesulfonamide

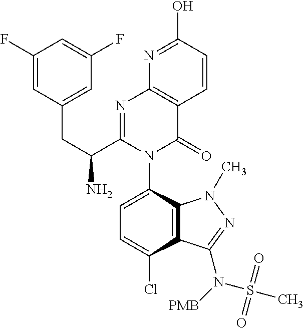

### Synthesis Scheme:

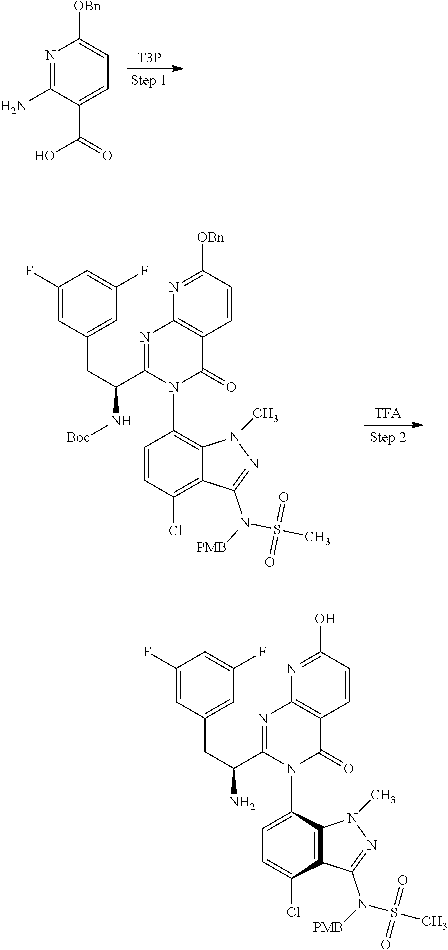

### Step 1:

To a suspension of (S)-2-((tert-butoxycarbonyl)amino)-3-(3,5-difluorophenyl)propanoic acid (5.49 g, 18.23 mmol) and 2-amino-6-(benzyloxy)nicotinic acid (4.45 g, 18.23 mmol) in acetonitrile (92 mL) (yellow solution) at −25° C. was added pyridine (9.83 mL, 122 mmol) followed by 2,4,6-tripropyl-1,3,5,2,4,6-trioxatriphosphinane 2,4,6-trioxide (“T3P”, 45.2 ml, 76 mmol). The reaction mixture (became a clear solution after T3P addition) was stirred at −25° C. to 10° C. over 4.5 h, then N-(7-amino-4-chloro-1-methyl-1H-indazol-3-yl)-N-(4-methoxybenzyl)methanesulfonamide (6 g, 15.19 mmol) was added and the mixture was stirred for 18 h while warming to rt. The reaction mixture was diluted with ethyl acetate, washed with 1N NaOH, then water, then 0.5 M citric acid, then water, then dried over Na₂SO₄ and concentrated in vacuo. The resulting residue was purified on silica (330 g RediSep Gold column) using 0-60% ethyl acetate in hexanes over 15 CV, then holding at 60% EtOAc for 10 CV. The desired fractions were pooled and concentrated to afford a pale yellow solid (8.1 g, 9.14 mmol, 60.1% yield), a mixture of tert-butyl N-[(1S)-1-[(3P,3P)-7-(benzyloxy)-3-(4-chloro-3-{N-[(4-methoxyphenyl)methyl]methanesulfonamido}-1-methyl-1H-indazol-7-yl)-4-oxo-3H,4H-pyrido[2,3-d]pyrimidin-2-yl]-2-(3,5-difluorophenyl)ethyl]carbamate (major) and tert-butyl N-[(1S)-1-[(3M,3M)-7-(benzyloxy)-3-(4-chloro-3-{N-[(4-methoxyphenyl)methyl]methanesulfonamido}-1-methyl-1H-indazol-7-yl)-4-oxo-3H,4H-pyrido[2,3-d]pyrimidin-2-yl]-2-(3,5-difluorophenyl)ethyl]carbamate (minor). LC/MS: m/z=886.25 [M+1]⁺.

### Step 2:

TFA (21.1 mL, 274 mmol) was added to a solution of tert-butyl (S)-(1-(7-(benzyloxy)-3-(4-chloro-3-(N-(4-methoxybenzyl)methylsulfonamido)-1-methyl-1H-indazol-7-yl)-4-oxo-3,4-dihydropyrido[2,3-d]pyrimidin-2-yl)-2-(3,5-difluorophenyl)ethyl)carbamate (Product from Step 1, 8.1 g, 9.14 mmol) in dichloromethane (45.7 mL). The mixture was stirred at rt for 2 h. The resultant pale yellow solution was concentrated. The residue was taken up in ethyl acetate, then washed three times with 1 N NaOH, then dried over Na₂SO₄ and then concentrated in vacuo to afford an oily residue. The residue was purified on silica gel (330 g RediSep Gold column) by a gradient method of Solvent A:Solvent B 65:35->0:100 (2 CV), then 0:100 (9 CV); Solvent A=hexanes; Solvent B=9:9:2 hexanes:ethyl acetate:MeOH. The first eluting isomer (major) was collected and concentrated in vacuo to afford N-[(6P)-7-{2-[(1S)-1-amino-2-(3,5-difluorophenyl)ethyl]-7-hydroxy-4-oxo-3H,4H-pyrido[2,3-d]pyrimidin-3-yl}-4-chloro-1-methyl-1H-indazol-3-yl]-N-[(4-methoxyphenyl)methyl]methanesulfonamide (4.1 g, 5.89 mmol, 64.5% yield). ¹H NMR (500 MHz, DMSO-d6) δ 7.86-7.98 (m, 1H) 7.15-7.37 (m, 4H) 6.97-7.06 (m, 1H) 6.70-6.89 (m, 4H) 6.40-6.48 (m, 1H) 4.70-4.88 (m, 2H) 3.41-3.81 (m, 7H) 3.20-3.28 (m, 1H) 3.08-3.12 (m, 3H) 2.71-2.79 (m, 1H) 1.69-2.00 (m, 2H). LC/MS: m/z=696.20 [M+1]⁺.

### Preparation of N—((S)-1-((3P)-3-(4-chloro-3-(N-(4-methoxybenzyl)methylsulfonamido)-1-methyl-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

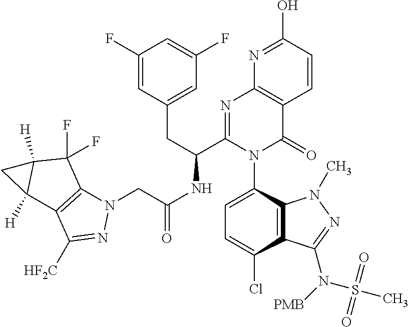

To a stirred solution of N-[(6P)-7-{2-[(1S)-1-amino-2-(3,5-difluorophenyl)ethyl]-7-hydroxy-4-oxo-3H,4H-pyrido[2,3-d]pyrimidin-3-yl}-4-chloro-1-methyl-1H-indazol-3-yl]-N-[(4-methoxyphenyl)methyl]methanesulfonamide (0.926 g, 1.330 mmol) in DMF (13 ml) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.351 g, 1.330 mmol), 2-(3H-[1,2,3]triazolo[4,5-b]pyridin-3-yl)-1,1,3,3-tetramethylisouronium hexafluorophosphate(V) (“HATU”, 0.531 g, 1.397 mmol), and DIPEA (0.581 ml, 3.33 mmol). The reaction mixture was stirred for 2 h after which the reaction mixture was diluted with water and extracted with ethyl acetate. The combined EtOAc extractions were washed with brine, dried over Na₂SO₄, and concentrated in vacuo. The crude product was purified via silica gel flash chromatography using 10-100% ethyl acetate in hexanes to provide N—((S)-1-((3P)-3-(4-chloro-3-(N-(4-methoxybenzyl)methylsulfonamido)-1-methyl-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.1 g, 88%) as an off-white foamy solid. LC/MS: m/z=942.25 [M+1]⁺.

### Preparation of N—((S)-1-((3P)-3-(4-chloro-1-methyl-3-(methylsulfonamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

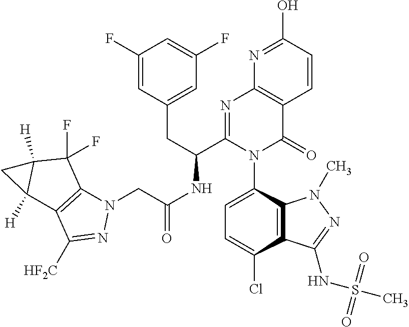

To a solution of N—((S)-1-((3P)-3-(4-chloro-3-(N-(4-methoxybenzyl)methylsulfonamido)-1-methyl-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (0.05 g, 0.053 mmol) in DCM (1 mL) and TFA (0.250 mL) was added triflic acid (0.014 mL, 0.159 mmol). The resultant purple solution was stirred for 1 h and then concentrated in vacuo. The crude residue was taken up in ethyl acetate and washed with saturated aqueous NaHCO3. The organic layer was concentrated and then purified by reverse phase preparative chromatography. Purification Conditions: Column: Waters Xterra C18, 19×100 mm, 10 m particles; Solvent A=0.1% NH4H in 100% Water. Solvent B=Acetonitrile. Flow Rate=40 mL/min. Start % B=37.6. Final % B=57.6. Gradient Time=6 min, then a 2 min hold at 98% B. Wavelength=215 and 254 nm. ESI+Range: 150 to 1500 dalton. Sample was loaded at 25% B. The product was isolated (26 mg, 59%). ¹H NMR (500 MHz, METHANOL-d4) δ 8.09-8.17 (m, 1H) 7.27-7.32 (m, 1H) 7.16-7.21 (m, 1H) 6.58-6.85 (m, 5H) 4.81-4.83 (m, 2H) 4.42-4.47 (m, 2H) 3.65-3.70 (m, 3H) 3.43-3.49 (m, 1H) 3.23-3.27 (m, 3H) 3.06-3.14 (m, 1H) 2.41-2.50 (m, 2H) 1.35-1.41 (m, 1H) 0.96-1.02 (m, 1H). LC/MS retention time=1.15 min; m/z=822.6 [M+H]⁺ (Column: Acquity UPLC BEH C18, 2.1×30 mm, 1.7 m particles; Solvent A=0.10% Formic acid in 100% Water. Solvent B=0.1% Formic Acid in 100% Acetonitrile. Flow Rate=0.8 mL/min. Start % B=5. Final % B=95. Gradient Time=1.6 min, then a 0.25 min hold at 95% B. Wavelength=215 nm.

### Preparation of (3P)-3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate

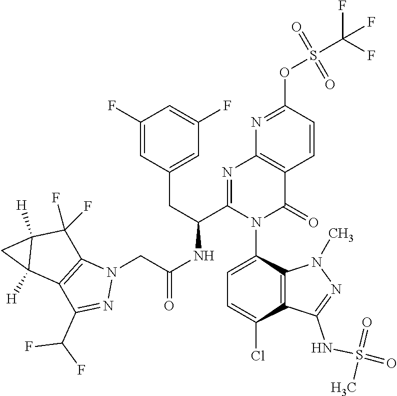

To a solution of N—((S)-1-((3P)-3-(4-chloro-1-methyl-3-(methylsulfonamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (2.12 g, 2.58 mmol) and 1,1,1-trifluoro-N-(pyridin-2-yl)-N-((trifluoromethyl)sulfonyl)methanesulfonamide (1.940 g, 5.42 mmol) in dichloromethane (12.9 mL) was added triethylamine (0.76 mL, 5.42 mmol) and the mixture was stirred at rt for 18 h. The reaction mixture was then directly subjected to silica gel chromatography (120 g RediSep column) eluting with 0-60% ethyl acetate in hexanes over 10 CV, then at 60% ethyl acetate in hexanes for 8 CV. The desired fractions were pooled and then concentrated under reduced pressure to afford (3P)-3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (1.6 g, 1.677 mmol, 65.0% yield) as an off-white solid foam. LC/MS: m/z=955.95 [M+1]⁺.

The general procedures, general analytical methods, and general purification methods used to prepare examples 2-40 are described below. The experimental procedure supplied for each specific example identifies the general method used to prepare and purify that compound.

### General Procedure A:

To 5 mL vial equipped with a stir bar was added a solution of 3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (30 mg, 0.031 mmol) in THF (1.0 mL), a solution of K₃PO₄ (0.025 g, 0.094 mmol) in water (0.25 mL), dichloro[9,9-dimethyl-4,5-bis(diphenylphosphino)xanthene]palladium(II) (2.377 mg, 3.14 μmol), and the appropriate boronic acid (0.094 mmol). The vial was degassed (the flask was evaluated and the atmosphere replaced with Ar; this process repeated three time) and then maintained under Ar atmosphere. The mixture was stirred at rt for 16 h. To the mixture was added 2 M ammonia in methanol (1 mL). The mixture was stirred for 2 h and then concentrated under reduced pressure. The resulting residue was dissolved in DMF, the solution was filtered, and the filtrate was subjected to prep-HPLC purification to afford the product as indicated.

### HPLC Purification:

HPLC purification was performed using one of the conditions indicated below, optionally followed by a second HPLC purification using a different condition indicated below. Based on analytical HPLC data obtained on the crude reaction mixture, the purification condition was optimized for each target compound by modifying the initial Solvent A:Solvent B ratio, the gradient time, the final Solvent A:Solvent B ratio, and the hold time at the final Solvent A:Solvent B concentration.

HPLC Condition A: Column: Zorbax Eclipse Plus C18, 21.2×100 mm, 5 m particles; Solvent A=0.1% Formic Acid in 100% Water. Solvent B=Acetonitrile. Flow Rate=40 mL/min. Wavelength=215 and 254 nm. ESI+Range: 150 to 1500 dalton.

HPLC Condition B: Column: Sunfire prep C18 OBD, 30×100 mm, 5 m particles; Solvent A: water:MeCN 95:5 w/0.1% TFA, Solvent B: MeCN:water 95:5 w/0.1% TFA. Flow Rate=42 mL/min. Wavelength=220 and 254 nm.

HPLC Condition C: Column: Waters Xterra C18, 19×100 mm, 10 m particles; Solvent A=0.1% NH₄₀H in 100% Water. Solvent B=Acetonitrile. Flow Rate=40 mL/min. Wavelength=215 and 254 nm. ESI+Range: 150 to 1500 dalton.

General LCMS Analysis Methods:

LCMS Method C:

Column: Acquity UPLC BEH C18, 2.1×30 mm, 1.7 m particles; Solvent A=0.1% Formic acid in 100% Water. Solvent B=0.10% Formic Acid in 100% Acetonitrile. Flow Rate=0.8 mL/min. Start % B=5. Final % B=95. Gradient Time=1.6 min, then a 0.25 min hold at 95% B. Wavelength=215 nm.

LCMS Method F:

Column: Acquity BEH C18, 2.1×30 mm, 1.7 m particles; Solvent A=0.1% Formic acid in 100% Water. Solvent B=0.1% Formic Acid in 100% Acetonitrile. Flow Rate=0.8 mL/min. Start % B=5. Final % B=95. Gradient Time=1.7 min, then a 0.2 min hold at 95% B. Wavelength=215 and 254 nm.

### Preparation of Example 3: N—((S)-1-((3P)-3-(4-chloro-1-methyl-3-(methylsulfonamido)-1H-indazol-7-yl)-7-(2,6-difluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

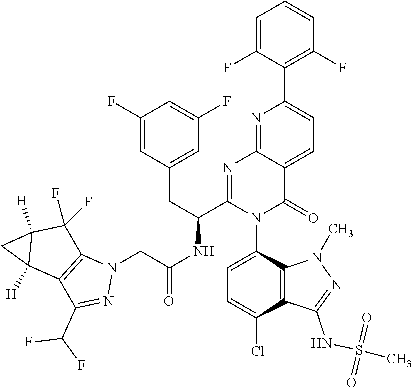

The title compound was prepared according to General Procedure A using (2,6-difluorophenyl)boronic acid as the coupling partner. The experiment afforded the title compound, N—((S)-1-((3P)-3-(4-chloro-1-methyl-3-(methylsulfonamido)-1H-indazol-7-yl)-7-(2,6-difluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method C: retention time=1.43 min.; observed ion=918.4 (M+H). 1H NMR (500 MHz, METHANOL-d4) δ ppm 8.66-8.78 (m, 1H) 7.70-7.82 (m, 1H) 7.43-7.59 (m, 1H) 7.05-7.29 (m, 4H) 6.39-6.73 (m, 4H) 4.32-4.54 (m, 2H) 3.54-3.61 (m, 3H) 3.34-3.42 (m, 1H) 3.12-3.17 (m, 3H) 2.99-3.07 (m, 1H) 2.24-2.35 (m, 2H) 1.21-1.27 (m, 1H) 0.84-0.92 (m, 1H)

### Preparation of Example 4: N—((S)-1-((3P)-3-(4-chloro-1-methyl-3-(methylsulfonamido)-1H-indazol-7-yl)-7-(2,3-difluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

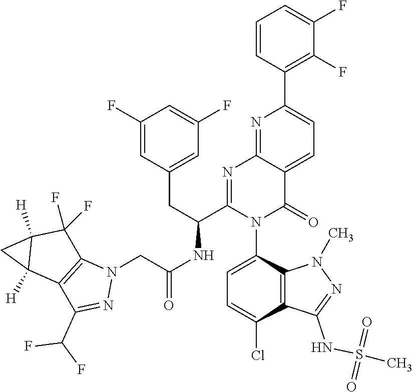

The title compound was prepared according to General Procedure A using (2,3-difluorophenyl)boronic acid as the coupling partner. The experiment afforded the title compound, N—((S)-1-((3P)-3-(4-chloro-1-methyl-3-(methylsulfonamido)-1H-indazol-7-yl)-7-(2,3-difluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.52 min.; observed ion=918.3 (M+H). 1H NMR (500 MHz, METHANOL-d4) δ ppm 8.63-8.73 (m, 1H) 8.00-8.07 (m, 1H) 7.76-7.85 (m, 1H) 7.17-7.34 (m, 4H) 6.41-6.73 (m, 4H) 4.42-4.54 (m, 2H) 3.54-3.58 (m, 3H) 3.37-3.43 (m, 1H) 3.14-3.15 (m, 3H) 3.03-3.08 (m, 1H) 2.27-2.35 (m, 2H) 1.22-1.26 (m, 1H) 0.87-0.92 (m, 1H)

### Preparation of Example 5: N—((S)-1-((3P)-3-(4-chloro-1-methyl-3-(methylsulfonamido)-1H-indazol-7-yl)-7-(4-fluoro-3-(hydroxymethyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

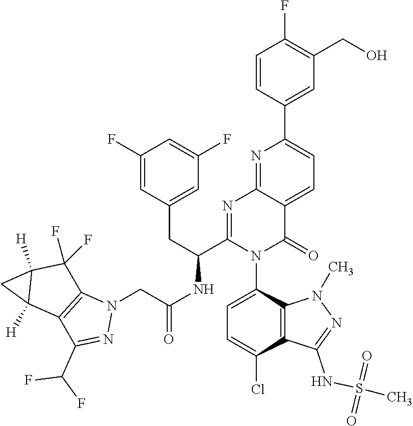

The title compound was prepared according to General Procedure A using (4-fluoro-3-(hydroxymethyl)phenyl)boronic acid as the coupling partner. The experiment afforded the title compound, N—((S)-1-((3P)-3-(4-chloro-1-methyl-3-(methylsulfonamido)-1H-indazol-7-yl)-7-(4-fluoro-3-(hydroxymethyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.36 min.; observed ion=930.4 (M+H). 1H NMR (500 MHz, METHANOL-d4) δ ppm 8.72-8.77 (m, 1H) 8.21 (d, J=8.35 Hz, 1H) 8.01-8.12 (m, 2H) 7.68-7.76 (m, 1H) 7.23-7.33 (m, 2H) 6.53-6.84 (m, 4H) 4.79 (s, 2H) 4.52-4.65 (m, 3H) 3.63 (s, 3H) 3.50 (dd, J=14.01, 4.47 Hz, 1H) 3.22 (s, 3H) 3.12-3.18 (m, 1H) 2.37-2.44 (m, 2H) 1.31-1.37 (m, 1H) 0.96-1.01 (m, 1H)

### Preparation of Example 6: N—((S)-1-((3P)-3-(4-chloro-1-methyl-3-(methylsulfonamido)-1H-indazol-7-yl)-7-(5-chloro-2,4-difluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

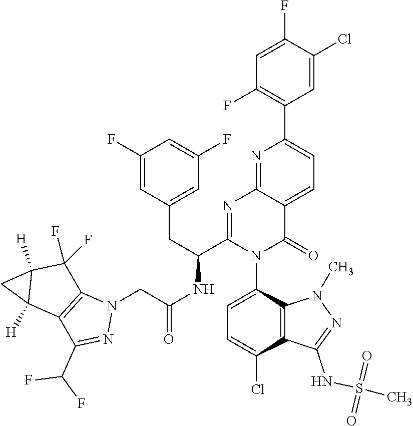

The title compound was prepared according to General Procedure A using (5-chloro-2,4-difluorophenyl)boronic acid as the coupling partner. The experiment afforded the title compound, N—((S)-1-((3P)-3-(4-chloro-1-methyl-3-(methylsulfonamido)-1H-indazol-7-yl)-7-(5-chloro-2,4-difluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.6 min.; observed ion=952.2 (M+H). 1H NMR (500 MHz, METHANOL-d4) δ ppm 8.73-8.82 (m, 1H) 8.35 (t, J=8.05 Hz, 1H) 8.10 (dd, J=8.35, 2.09 Hz, 1H) 7.44 (dd, J=10.73, 9.24 Hz, 1H) 7.24-7.36 (m, 2H) 6.51-6.83 (m, 4H) 4.54-4.67 (m, 2H) 3.63 (s, 3H) 3.49 (dd, J=14.46, 4.62 Hz, 1H) 3.22 (s, 3H) 3.11-3.18 (m, 1H) 2.37-2.45 (m, 2H) 1.32-1.37 (m, 1H) 0.96-1.01 (m, 1H)

### Preparation of Example 7: N—((S)-1-((3P)-3-(4-chloro-3-(cyclopropanesulfonamido)-1-(2,2-difluoroethyl)-1H-indazol-7-yl)-7-(2-fluorophenyl)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide

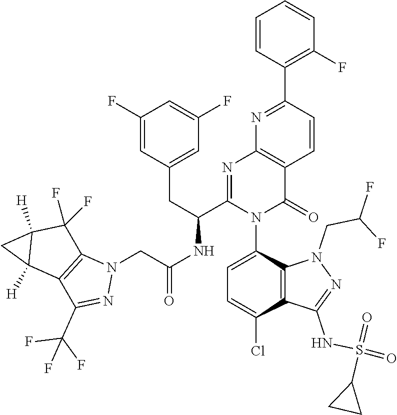

To a stirred solution of (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-(2,2-difluoroethyl)-1H-indazol-3-yl)-N-(4-methoxybenzyl)cyclopropanesulfonamide (0.04 g, 0.047 mmol) in N,N-dimethylformamide (1 mL) was added 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid (0.013 g, 0.047 mmol), 2-(3H-[1,2,3]triazolo[4,5-b]pyridin-3-yl)-1,1,3,3-tetramethylisouronium hexafluorophosphate(V) (“HATU”, 0.021 g, 0.056 mmol) and DIPEA (0.021 mL, 0.118 mmol). The solution was stirred for 18 h, then the reaction solution was concentrated under reduced pressure. The residue was taken up in DCM/TFA (1:1, 2 mL), then to the solution was added triflic acid (0.017 mL, 0.188 mmol). The resulting purple solution was stirred for 1 h. The solution was concentrated under reduced pressure and the resulting residue was dissolved in DCM (1.5 mL). The organic solution was washed with sat. aq. NaHCO₃ (1 mL) and then was concentrated under reduced pressure. The residue was dissolved in DMF and the resulting solution was then subjected to prep-HPLC purification to afforded the title compound, N—((S)-1-((3P)-3-(4-chloro-3-(cyclopropanesulfonamido)-1-(2,2-difluoroethyl)-1H-indazol-7-yl)-7-(2-fluorophenyl)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide. The sample was analyzed using LCMS Method F: retention time=1.56 min.; observed ion=994.4 (M+H). 1H NMR (500 MHz, METHANOL-d4) δ ppm 8.74-8.83 (m, 1H) 8.09-8.17 (m, 2H) 7.60-7.68 (m, 1H) 7.34-7.49 (m, 4H) 5.92-6.85 (m, 4H) 4.66-4.84 (m, 3H) 4.32-4.49 (m, 1H) 3.94-4.08 (m, 1H) 3.42-3.46 (m, 1H) 3.09-3.16 (m, 1H) 2.90-2.97 (m, 1H) 2.41-2.50 (m, 2H) 1.35-1.42 (m, 1H) 0.96-1.14 (m, 5H)

### Preparation of Example 8: N—((S)-1-((3P)-3-(4-chloro-1-methyl-3-(methylsulfonamido)-1H-indazol-7-yl)-7-(4-chloro-2-fluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

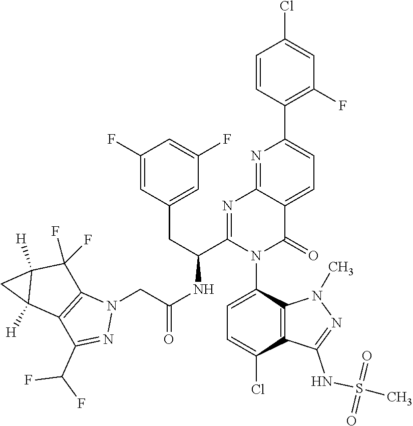

The title compound was prepared according to General Procedure A using (4-chloro-2-fluorophenyl)boronic acid as the coupling partner. The experiment afforded the title compound, N—((S)-1-((3P)-3-(4-chloro-1-methyl-3-(methylsulfonamido)-1H-indazol-7-yl)-7-(4-chloro-2-fluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.59 min.; observed ion=934.2 (M+H). H NMR (500 MHz, METHANOL-d4) δ ppm 8.77 (s, 1H) 8.05-8.19 (m, 2H) 7.45-7.52 (m, 2H) 7.25-7.33 (m, 2H) 6.53-6.82 (m, 4H) 4.52-4.65 (m, 2H) 3.63 (s, 3H) 3.49 (dd, J=14.16, 4.62 Hz, 1H) 3.22 (s, 3H) 3.11-3.18 (m, 1H) 2.40 (td, J=7.67, 3.73 Hz, 2H) 1.30-1.38 (m, 1H) 0.95-1.04 (m, 1H)

### Preparation of Example 9: N—((S)-1-((3P)-3-(4-chloro-1-methyl-3-(methylsulfonamido)-1H-indazol-7-yl)-7-(2,5-difluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

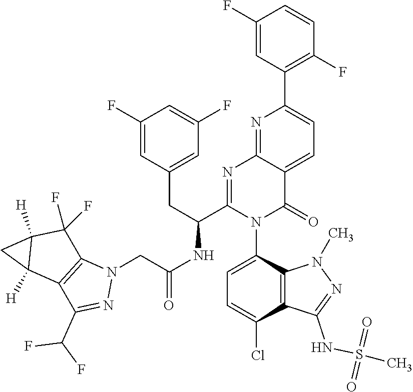

The title compound was prepared according to General Procedure A using (2,5-difluorophenyl)boronic acid as the coupling partner. The experiment afforded the title compound, N—((S)-1-((3P)-3-(4-chloro-1-methyl-3-(methylsulfonamido)-1H-indazol-7-yl)-7-(2,5-difluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.52 min.; observed ion=918.3 (M+H). 1H NMR (500 MHz, METHANOL-d4) δ ppm 8.61-8.71 (m, 1H) 8.07-8.16 (m, 1H) 7.80-7.88 (m, 2H) 6.87-7.28 (m, 5H) 6.47-6.72 (m, 4H) 4.42-4.54 (m, 2H) 3.54-3.58 (m, 3H) 3.37-3.43 (m, 1H) 3.13-3.16 (m, 3H) 3.03-3.09 (m, 1H) 2.27-2.35 (m, 2H) 1.22-1.27 (m, 1H) 0.86-0.91 (m, 1H)

### Preparation of Example 10: N—((S)-1-((3P)-3-(4-chloro-1-(2,2-difluoroethyl)-3-(methylsulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

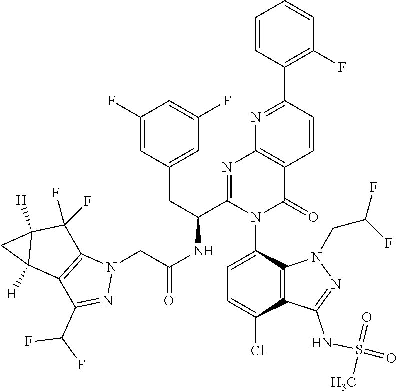

To a stirred solution of (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-(2,2-difluoroethyl)-1H-indazol-3-yl)methanesulfonamide (0.055 g, 0.078 mmol) in tetrahydrofuran (1.562 ml) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.022 g, 0.082 mmol), 2-(3H-[1,2,3]triazolo[4,5-b]pyridin-3-yl)-1,1,3,3-tetramethylisouronium hexafluorophosphate(V) (“HATU”, 0.036 g, 0.094 mmol) and DIPEA (0.034 ml, 0.195 mmol). The reaction mixture was stirred for 18 h at rt and then was concentrated under reduced pressure. The residue was taken up in DMF (2 mL), the solution was filtered, and the filtrate was subjected to prep-HPLC purification to afforded the title compound, N—((S)-1-((3P)-3-(4-chloro-1-(2,2-difluoroethyl)-3-(methylsulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.42 min.; observed ion=950.3 (M+H). 1H NMR (500 MHz, METHANOL-d4) δ ppm 8.74-8.84 (m, 1H) 8.08-8.16 (m, 2H) 7.61-7.68 (m, 1H) 7.34-7.48 (m, 4H) 5.92-6.84 (m, 5H) 4.76-4.84 (m, 1H) 4.64-4.76 (m, 2H) 4.37-4.50 (m, 1H) 3.96-4.08 (m, 1H) 3.42-3.50 (m, 1H) 3.26-3.30 (m, 3H) 3.09-3.17 (m, 1H) 2.38-2.47 (m, 2H) 1.31-1.39 (m, 1H) 0.97-1.05 (m, 1H)

### Preparation of Example 11: N—((S)-1-((3P)-3-(4-chloro-3-(methylsulfonamido)-1-(2,2,2-trifluoroethyl)-1H-indazol-7-yl)-7-(2-fluorophenyl)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide

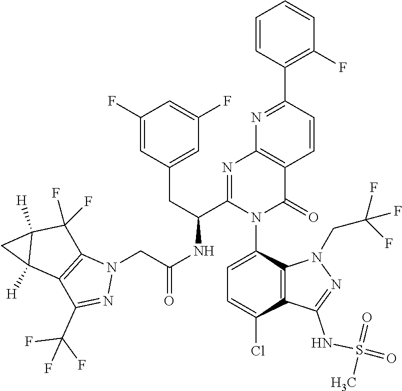

To a stirred solution of (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-(2,2,2-trifluoroethyl)-1H-indazol-3-yl)methanesulfonamide (0.04 g, 0.055 mmol) in N,N-Dimethylformamide (1 mL) was added 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid (0.016 g, 0.055 mmol), 2-(3H-[1,2,3]triazolo[4,5-b]pyridin-3-yl)-1,1,3,3-tetramethylisouronium hexafluorophosphate(V) (“HATU”, 0.025 g, 0.066 mmol) and DIPEA (0.024 mL, 0.138 mmol). The reaction mixture was stirred at room temperature for 1 h, then was diluted DMF (1 mL), the solution was filtered, and the filtrate was subjected to prep-HPLC afforded the title compound, N—((S)-1-((3P)-3-(4-chloro-3-(methylsulfonamido)-1-(2,2,2-trifluoroethyl)-1H-indazol-7-yl)-7-(2-fluorophenyl)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide. The sample was analyzed using LCMS Method F: retention time=1.52 min.; observed ion=986.4 (M+H). 1H NMR (500 MHz, METHANOL-d4) δ ppm 8.71-8.82 (m, 1H) 8.09-8.16 (m, 2H) 7.60-7.68 (m, 1H) 7.31-7.51 (m, 4H) 6.76-6.84 (m, 1H) 6.47-6.55 (m, 2H) 4.69-4.85 (m, 4H) 4.22-4.36 (m, 1H) 3.38-3.44 (m, 1H) 3.25-3.28 (m, 3H) 3.04-3.14 (m, 1H) 2.41-2.54 (m, 2H) 1.36-1.46 (m, 1H) 1.02-1.10 (m, 1H)

### Preparation of Example 12: N—((S)-1-((3P)-3-(4-chloro-1-methyl-3-(methylsulfonamido)-1H-indazol-7-yl)-4-oxo-7-(3,4,5-trifluorophenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

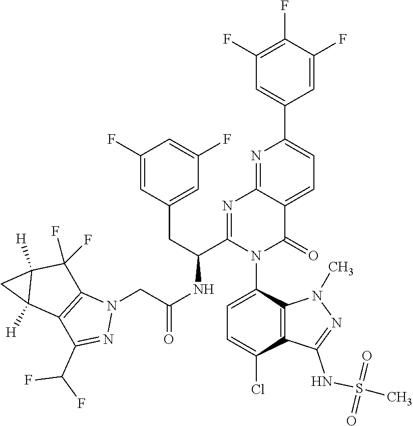

The title compound was prepared according to General Procedure A using (3,4,5-trifluorophenyl)boronic acid as the coupling partner. The experiment afforded the title compound, N—((S)-1-((3P)-3-(4-chloro-1-methyl-3-(methylsulfonamido)-1H-indazol-7-yl)-4-oxo-7-(3,4,5-trifluorophenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.55 min.; observed ion=937.4 (M+H). 1H NMR (500 MHz, METHANOL-d4) δ ppm 8.72-8.90 (m, 1H) 8.07-8.28 (m, 3H) 7.28-7.39 (m, 2H) 6.53-6.86 (m, 4H) 4.52-4.67 (m, 2H) 3.64-3.68 (m, 3H) 3.49-3.55 (m, 1H) 3.26-3.27 (m, 3H) 3.16-3.20 (m, 1H) 2.36-2.48 (m, 2H) 1.34-1.41 (m, 1H) 0.97-1.06 (m, 1H)

### Preparation of Example 13: N—((S)-1-((3P)-3-(4-chloro-1-methyl-3-(methylsulfonamido)-1H-indazol-7-yl)-7-(4-chlorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

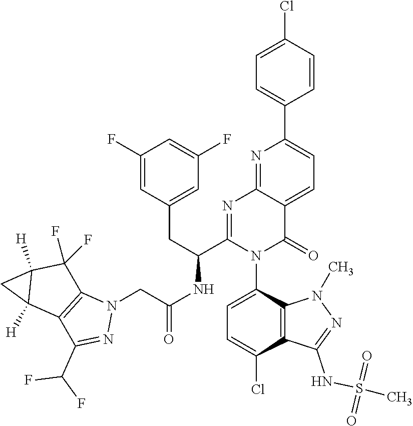

The title compound was prepared according to General Procedure A using (4-chlorophenyl)boronic acid as the coupling partner. The experiment afforded the title compound, N—((S)-1-((3P)-3-(4-chloro-1-methyl-3-(methylsulfonamido)-1H-indazol-7-yl)-7-(4-chlorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. 1H NMR (500 MHz, METHANOL-d4) δ ppm 8.64-8.74 (m, 1H) 7.79-7.89 (m, 1H) 7.58-7.69 (m, 2H) 7.43-7.55 (m, 1H) 7.15-7.26 (m, 2H) 6.41-6.72 (m, 4H) 4.38-4.53 (m, 2H) 3.53-3.59 (m, 3H) 3.34-3.43 (m, 1H) 3.14-3.16 (m, 3H) 3.00-3.06 (m, 1H) 2.26-2.34 (m, 2H) 1.22-1.27 (m, 1H) 0.86-0.91 (m, 1H)

### Preparation of Example 14: N—((S)-1-((3P)-3-(4-chloro-1-methyl-3-(methylsulfonamido)-1H-indazol-7-yl)-4-oxo-7-(4-(trifluoromethyl)phenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

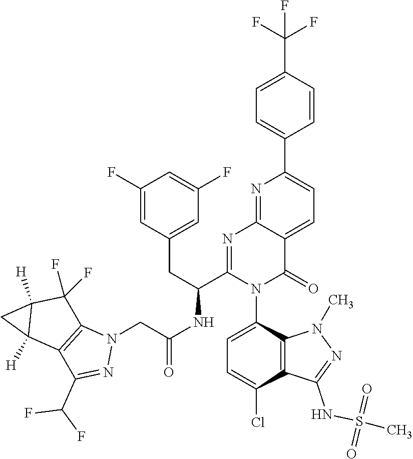

The title compound was prepared according to General Procedure A using (4-(trifluoromethyl)phenyl)boronic acid as the coupling partner. The experiment afforded the title compound, N—((S)-1-((3P)-3-(4-chloro-1-methyl-3-(methylsulfonamido)-1H-indazol-7-yl)-4-oxo-7-(4-(trifluoromethyl)phenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.58 min.; observed ion=950.3 (M−H). 1H NMR (500 MHz, METHANOL-d4) δ ppm 8.59-8.68 (m, 1H) 8.34-8.42 (m, 1H) 8.04-8.19 (m, 2H) 7.34-7.44 (m, 1H) 7.11-7.26 (m, 2H) 6.42-6.75 (m, 4H) 4.39-4.54 (m, 2H) 3.53-3.56 (m, 3H) 3.36-3.44 (m, 1H) 3.11-3.16 (m, 3H) 3.01-3.09 (m, 1H) 2.23-2.35 (m, 2H) 1.21-1.27 (m, 1H) 0.82-0.94 (m, 1H)

### Preparation of Example 15: N—((S)-1-((3P)-3-(4-chloro-1-methyl-3-(methylsulfonamido)-1H-indazol-7-yl)-4-oxo-7-(2,3,4-trifluorophenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

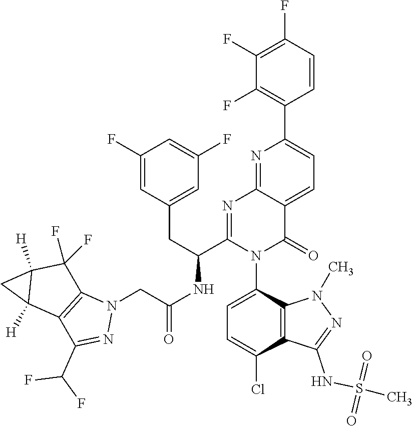

The title compound was prepared according to General Procedure A using (2,3,4-trifluorophenyl)boronic acid as the coupling partner. The experiment afforded the title compound, N—((S)-1-((3P)-3-(4-chloro-1-methyl-3-(methylsulfonamido)-1H-indazol-7-yl)-4-oxo-7-(2,3,4-trifluorophenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.54 min.; observed ion=936.3 (M+H). 1H NMR (500 MHz, METHANOL-d4) δ ppm 8.78-8.85 (m, 1H) 8.06-8.11 (m, 1H) 7.94-8.01 (m, 1H) 7.38-7.47 (m, 1H) 7.25-7.36 (m, 3H) 6.54-6.86 (m, 4H) 4.52-4.69 (m, 2H) 3.64-3.70 (m, 3H) 3.52 (dd, J=14.16, 4.32 Hz, 1H) 3.25-3.27 (m, 3H) 3.13-3.19 (m, 1H) 2.39-2.46 (m, 2H) 1.34-1.40 (m, 1H) 0.98-1.05 (m, 1H)

### Preparation of Example 16: N—((S)-1-((3P)-3-(4-chloro-1-methyl-3-(methylsulfonamido)-1H-indazol-7-yl)-7-(4-fluoro-2-(hydroxymethyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

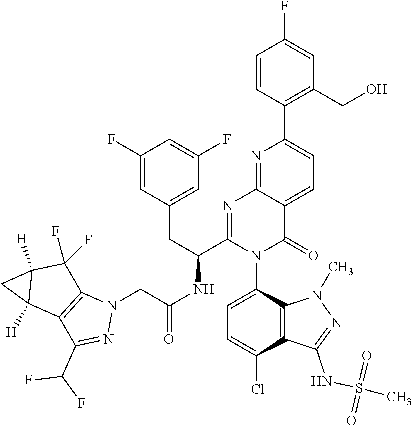

The title compound was prepared according to General Procedure A using (4-fluoro-2-(hydroxymethyl)phenyl)boronic acid as the coupling partner. The experiment afforded the title compound, N—((S)-1-((3P)-3-(4-chloro-1-methyl-3-(methylsulfonamido)-1H-indazol-7-yl)-7-(4-fluoro-2-(hydroxymethyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.4 min.; observed ion=930.4 (M+H). 1H NMR (500 MHz, METHANOL-d4) δ ppm 8.75-8.81 (m, 1H) 7.92 (d, J=8.35 Hz, 1H) 7.75 (dd, J=8.64, 5.66 Hz, 1H) 7.46 (dd, J=9.84, 2.68 Hz, 1H) 7.22-7.35 (m, 3H) 6.52-6.82 (m, 4H) 4.76 (s, 2H) 4.50-4.60 (m, 2H) 3.61-3.69 (m, 3H) 3.48 (dd, J=14.16, 4.62 Hz, 1H) 3.23 (s, 3H) 3.12 (dd, J=14.16, 9.69 Hz, 1H) 2.40 (ddd, J=11.25, 7.67, 4.02 Hz, 2H) 1.30-1.37 (m, 1H) 0.94-1.01 (m, 1H)

### Preparation of Example 17: N—((S)-1-((3P)-3-(4-chloro-1-methyl-3-(methylsulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide

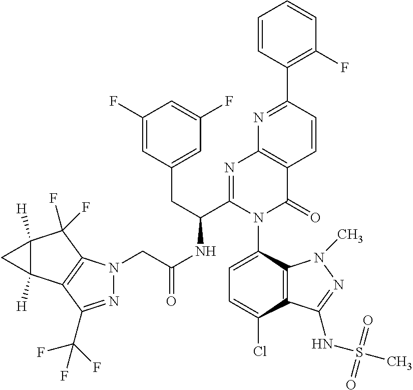

To a stirred solution of (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-methyl-1H-indazol-3-yl)methanesulfonamide (0.035 g, 0.053 mmol) in N,N-Dimethylformamide (1 mL) was added 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid (0.015 g, 0.053 mmol), 2-(3H-[1,2,3]triazolo[4,5-b]pyridin-3-yl)-1,1,3,3-tetramethylisouronium hexafluorophosphate(V) (“HATU”, 0.024 g, 0.064 mmol) and DIPEA (0.023 mL, 0.133 mmol). The reaction mixture was stirred at room temperature for 1 h, and then was diluted with DMF (1 mL) and purified by prep-HPLC to afford the title compound, N—((S)-1-((3P)-3-(4-chloro-1-methyl-3-(methylsulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide. The sample was analyzed using LCMS Method F: retention time=1.47 min.; observed ion=918.4 (M+H). 1H NMR (500 MHz, METHANOL-d4) δ ppm 8.71-8.78 (m, 1H) 8.05-8.12 (m, 2H) 7.58-7.65 (m, 1H) 7.25-7.45 (m, 4H) 6.74-6.81 (m, 1H) 6.57-6.64 (m, 2H) 4.56-4.71 (m, 3H) 3.61-3.66 (m, 3H) 3.46-3.53 (m, 1H) 3.23-3.24 (m, 3H) 3.11-3.18 (m, 1H) 2.38-2.50 (m, 2H) 1.33-1.40 (m, 1H) 1.00-1.07 (m, 1H)

### Preparation of Example 18: N—((S)-1-((3P)-3-(4-chloro-1-methyl-3-(methylsulfonamido)-1H-indazol-7-yl)-7-(4-fluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

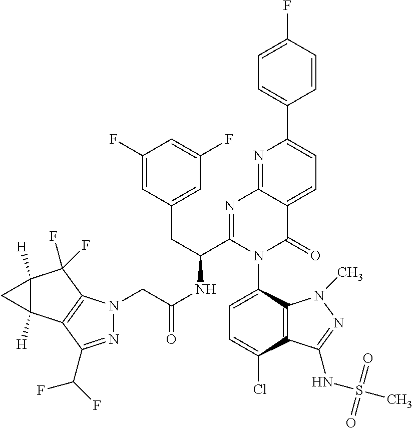

The title compound was prepared according to General Procedure A using (4-fluorophenyl)boronic acid as the coupling partner. The experiment afforded the title compound, N—((S)-1-((3P)-3-(4-chloro-1-methyl-3-(methylsulfonamido)-1H-indazol-7-yl)-7-(4-fluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.5 min.; observed ion=900.4 (M−H). 1H NMR (500 MHz, METHANOL-d4) δ ppm 8.58-8.69 (m, 1H) 8.16-8.25 (m, 2H) 8.03-8.11 (m, 1H) 7.41-7.54 (m, 1H) 7.14-7.28 (m, 4H) 7.01-7.10 (m, 1H) 6.42-6.74 (m, 4H) 4.39-4.53 (m, 2H) 3.53-3.56 (m, 3H) 3.37-3.43 (m, 1H) 3.13-3.16 (m, 3H) 3.02-3.08 (m, 1H) 2.26-2.34 (m, 2H) 1.21-1.27 (m, 1H) 0.86-0.93 (m, 1H)

### Preparation of Example 19: N—((S)-1-((3P)-3-(4-chloro-1-methyl-3-(methylsulfonamido)-1H-indazol-7-yl)-7-(4-fluoro-2-(methylsulfonyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

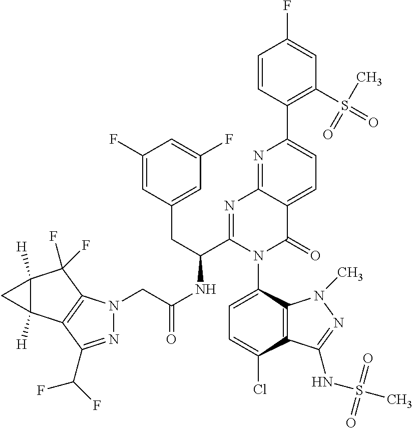

The title compound was prepared according to General Procedure A using (4-fluoro-2-(methylsulfonyl)phenyl)boronic acid as the coupling partner. The experiment afforded the title compound, N—((S)-1-((3P)-3-(4-chloro-1-methyl-3-(methylsulfonamido)-1H-indazol-7-yl)-7-(4-fluoro-2-(methylsulfonyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.4 min.; observed ion=978.3 (M−H). 1H NMR (500 MHz, METHANOL-d4) δ ppm 8.74-8.82 (m, 1H) 7.98-8.03 (m, 1H) 7.74-7.84 (m, 2H) 7.64-7.71 (m, 1H) 7.26-7.34 (m, 2H) 6.52-6.82 (m, 4H) 4.90-4.93 (m, 1H) 4.46-4.52 (m, 2H) 3.63-3.68 (m, 3H) 3.58-3.63 (m, 3H) 3.45-3.53 (m, 1H) 3.20-3.26 (m, 3H) 3.05-3.12 (m, 1H) 2.35-2.46 (m, 2H) 1.30-1.37 (m, 1H) 0.93-1.02 (m, 1H)

### Preparation of Example 20: N—((S)-1-((3P)-3-(4-chloro-3-(cyclopropanesulfonamido)-1-(2,2-difluoroethyl)-1H-indazol-7-yl)-7-(2-fluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

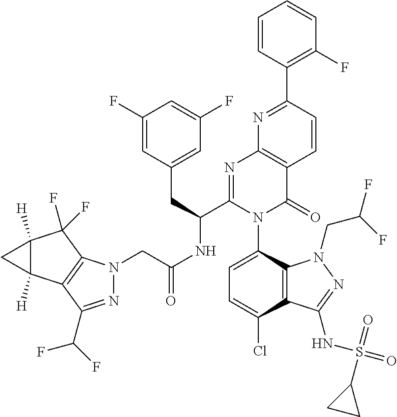

To a stirred solution of (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-(2,2-difluoroethyl)-1H-indazol-3-yl)-N-(4-methoxybenzyl)cyclopropanesulfonamide (0.04 g, 0.047 mmol) in N,N-Dimethylformamide (1 mL) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.012 g, 0.047 mmol), 2-(3H-[1,2,3]triazolo[4,5-b]pyridin-3-yl)-1,1,3,3-tetramethylisouronium hexafluorophosphate(V) (“HATU”, 0.021 g, 0.056 mmol) and DIPEA (0.021 mL, 0.118 mmol). The reaction solution was stirred for 18 h and then was concentrated under reduced pressure. The resulting residue was taken up in DCM/TFA (1:1, 2 mL). To the solution was added triflic acid (0.017 mL, 0.188 mmol). The purple solution was stirred for 1 h and then was concentrated under reduced pressure. The residue was taken up in DCM (1.5 mL), washed with sat. aq. NaHCO₃(1 mL), and then concentrated under reduced pressure. The residue was dissolved in DMF (2 mL) and then subjected to prep-HPLC purification to afford the title compound, N—((S)-1-((3P)-3-(4-chloro-3-(cyclopropanesulfonamido)-1-(2,2-difluoroethyl)-1H-indazol-7-yl)-7-(2-fluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.52 min.; observed ion=976.4 (M+H). 1H NMR (500 MHz, METHANOL-d4) δ ppm 8.73-8.82 (m, 1H) 8.08-8.18 (m, 2H) 7.60-7.68 (m, 1H) 7.34-7.49 (m, 4H) 5.90-6.85 (m, 5H) 4.60-4.81 (m, 3H) 4.36-4.51 (m, 1H) 3.93-4.08 (m, 1H) 3.40-3.49 (m, 1H) 3.07-3.16 (m, 1H) 2.87-2.97 (m, 1H) 2.37-2.47 (m, 2H) 1.27-1.40 (m, 2H) 1.05-1.15 (m, 2H) 0.99-1.03 (m, 2H)

### Preparation of Example 21: N—((S)-1-((3P)-3-(4-chloro-1-methyl-3-(methylsulfonamido)-1H-indazol-7-yl)-7-(3,5-difluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

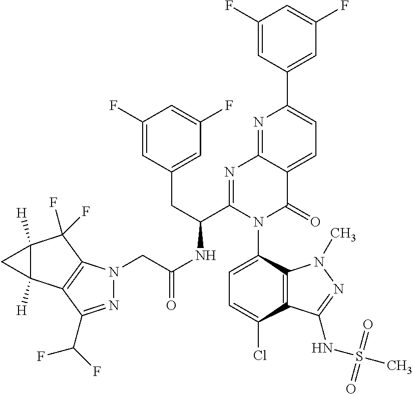

The title compound was prepared according to General Procedure A using (3,5-difluorophenyl)boronic acid as the coupling partner. The experiment afforded the title compound, N—((S)-1-((3P)-3-(4-chloro-1-methyl-3-(methylsulfonamido)-1H-indazol-7-yl)-7-(3,5-difluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.53 min.; observed ion=918.3 (M+H). 1H NMR (500 MHz, METHANOL-d4) δ ppm 8.74-8.87 (m, 1H) 8.05-8.21 (m, 2H) 7.47-7.54 (m, 2H) 7.27-7.38 (m, 2H) 6.54-6.85 (m, 4H) 4.50-4.64 (m, 2H) 3.66-3.69 (m, 3H) 3.49-3.55 (m, 1H) 3.26-3.27 (m, 3H) 3.14-3.19 (m, 1H) 2.39-2.46 (m, 2H) 1.34-1.39 (m, 1H) 0.98-1.04 (m, 1H)

### Preparation of Example 22: N—((S)-1-((3P)-3-(4-chloro-1-methyl-3-(methylsulfonamido)-1H-indazol-7-yl)-7-(3-chloro-2,4-difluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

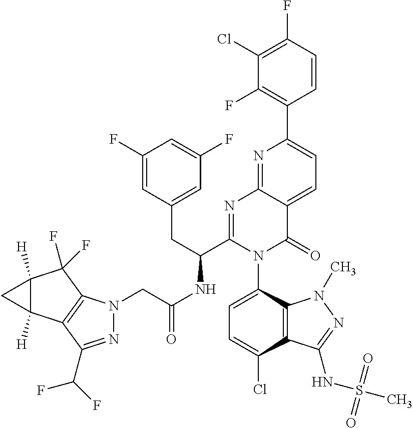

The title compound was prepared according to General Procedure A using (3-chloro-2,4-difluorophenyl)boronic acid as the coupling partner. The experiment afforded the title compound, N—((S)-1-((3P)-3-(4-chloro-1-methyl-3-(methylsulfonamido)-1H-indazol-7-yl)-7-(3-chloro-2,4-difluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. 1H NMR (500 MHz, METHANOL-d4) δ ppm 8.75-8.84 (m, 1H) 8.23-8.31 (m, 1H) 8.09-8.15 (m, 1H) 7.38-7.45 (m, 2H) 7.26-7.36 (m, 2H) 6.54-6.83 (m, 4H) 4.52-4.64 (m, 2H) 3.65-3.68 (m, 3H) 3.49-3.55 (m, 1H) 3.24-3.27 (m, 3H) 3.14-3.19 (m, 1H) 2.39-2.47 (m, 2H) 1.35-1.39 (m, 1H) 0.98-1.03 (m, 1H)

### Preparation of Example 23: N—((S)-1-((3P)-3-(4-chloro-1-methyl-3-(methylsulfonamido)-1H-indazol-7-yl)-7-(3-(methylsulfonyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

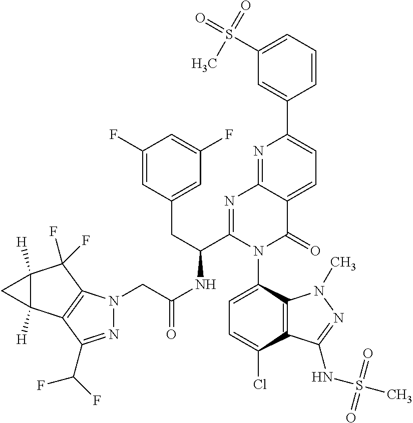

The title compound was prepared according to General Procedure A using (3-(methylsulfonyl)phenyl)boronic acid as the coupling partner. The experiment afforded the title compound, N—((S)-1-((3P)-3-(4-chloro-1-methyl-3-(methylsulfonamido)-1H-indazol-7-yl)-7-(3-(methylsulfonyl)phenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.35 min.; observed ion=960.4 (M+H). 1H NMR (500 MHz, METHANOL-d4) δ ppm 8.88-8.94 (m, 1H) 8.76-8.83 (m, 1H) 8.56-8.63 (m, 1H) 8.27-8.32 (m, 1H) 8.14-8.20 (m, 1H) 7.85-7.92 (m, 1H) 7.24-7.34 (m, 2H) 6.52-6.83 (m, 4H) 4.89-4.91 (m, 1H) 4.52-4.65 (m, 2H) 3.61-3.66 (m, 3H) 3.47-3.54 (m, 1H) 3.25 (s, 3H) 3.22 (s, 3H) 3.13-3.19 (m, 1H) 2.37-2.45 (m, 2H) 1.31-1.37 (m, 1H) 0.96-1.01 (m, 1H)

### Preparation of Example 24: N—((S)-1-((3P)-3-(4-chloro-1-methyl-3-(methylsulfonamido)-1H-indazol-7-yl)-7-(2,4-difluorophenyl)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide

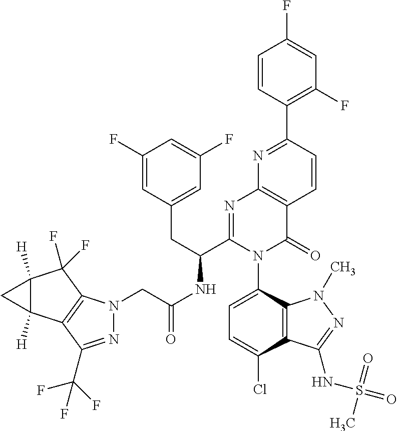

To a stirred solution of (S)—N-((6P)-7-(2-(1-amino-2-(3,5-difluorophenyl)ethyl)-7-(2,4-difluorophenyl)-4-oxopyrido[2,3-d]pyrimidin-3(4H)-yl)-4-chloro-1-methyl-1H-indazol-3-yl)-N-(4-methoxybenzyl)methanesulfonamide (0.05 g, 0.063 mmol) in tetrahydrofuran (1 ml) and N,N-Dimethylformamide (0.250 ml) was added 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid (0.018 g, 0.063 mmol), 2-(3H-[1,2,3]triazolo[4,5-b]pyridin-3-yl)-1,1,3,3-tetramethylisouronium hexafluorophosphate(V) (“HATU”, 0.029 g, 0.076 mmol) and DIPEA (0.028 ml, 0.158 mmol). The reaction mixture was stirred at rt for 18 h. The solution was concentrated under reduced pressure and the residue was taken up in DCM/TFA (1:1, 2 mL). To the solution was added triflic acid (0.017 mL, 0.188 mmol) and the resulting purple solution was stirred for 1 h. The solution was concentrated under reduced pressure and the residue was taken up in DCM (1.5 mL). The solution was washed with sat. aq. NaHCO₃(1 mL) and then was concentrated under reduced pressure. The residue was dissolved in DMF (2 mL) and then subjected to prep-HPLC purification to afford the title compound, N—((S)-1-((3P)-3-(4-chloro-1-methyl-3-(methylsulfonamido)-1H-indazol-7-yl)-7-(2,4-difluorophenyl)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide. The sample was analyzed using LCMS Method F: retention time=1.53 min.; observed ion=936.1 (M+H). 1H NMR (500 MHz, METHANOL-d4) δ ppm 8.62-8.70 (m, 1H) 8.04-8.13 (m, 1H) 7.92-7.99 (m, 1H) 7.08-7.25 (m, 4H) 6.64-6.72 (m, 1H) 6.45-6.55 (m, 2H) 4.73 (br s, 1H) 4.43-4.62 (m, 2H) 3.51-3.57 (m, 3H) 3.31-3.43 (m, 1H) 3.14-3.15 (m, 3H) 3.01-3.06 (m, 1H) 2.29-2.40 (m, 2H) 1.25-1.30 (m, 1H) 0.91-0.97 (m, 1H)

### Preparation of Example 25: N—((S)-1-((3P)-3-(4-chloro-1-methyl-3-(methylsulfonamido)-1H-indazol-7-yl)-7-(2,4-dichlorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

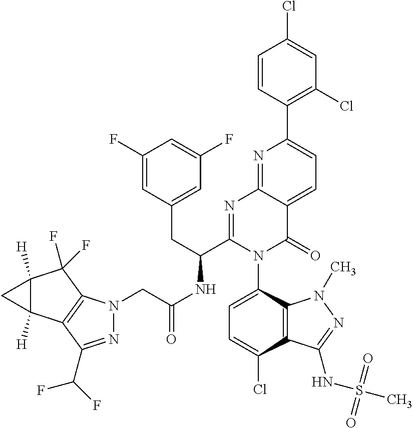

The title compound was prepared according to General Procedure A using (2,4-dichlorophenyl)boronic acid as the coupling partner. The experiment afforded the title compound, N—((S)-1-((3P)-3-(4-chloro-1-methyl-3-(methylsulfonamido)-1H-indazol-7-yl)-7-(2,4-dichlorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. 1H NMR (500 MHz, METHANOL-d4) δ ppm 8.67-8.74 (m, 1H) 7.95-8.02 (m, 1H) 7.71-7.80 (m, 1H) 7.37-7.46 (m, 1H) 7.28-7.35 (m, 1H) 7.17-7.24 (m, 2H) 6.43-6.71 (m, 4H) 4.39-4.53 (m, 2H) 3.53-3.58 (m, 3H) 3.34-3.45 (m, 1H) 3.14-3.15 (m, 3H) 3.01-3.08 (m, 1H) 2.27-2.35 (m, 2H) 1.22-1.27 (m, 1H) 0.86-0.92 (m, 1H)

### Preparation of Example 26: N—((S)-1-((3P)-3-(4-chloro-1-methyl-3-(methylsulfonamido)-1H-indazol-7-yl)-7-(2,4-difluoro-3-methoxyphenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

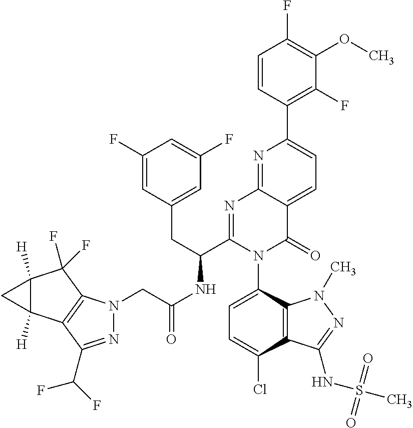

The title compound was prepared according to General Procedure A using (2,4-difluoro-3-methoxyphenyl)boronic acid as the coupling partner. The experiment afforded the title compound, N—((S)-1-((3P)-3-(4-chloro-1-methyl-3-(methylsulfonamido)-1H-indazol-7-yl)-7-(2,4-difluoro-3-methoxyphenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.51 min.; observed ion=948.3 (M+H). 1H NMR (500 MHz, METHANOL-d4) δ ppm 8.74-8.84 (m, 1H) 8.03-8.10 (m, 1H) 7.77-7.87 (m, 1H) 7.24-7.39 (m, 3H) 6.55-6.85 (m, 4H) 4.50-4.64 (m, 2H) 4.06-4.11 (m, 3H) 3.66-3.68 (m, 3H) 3.49-3.54 (m, 1H) 3.25-3.28 (m, 3H) 3.12-3.18 (m, 1H) 2.39-2.47 (m, 2H) 1.34-1.39 (m, 1H) 0.98-1.05 (m, 1H)

### Preparation of Example 27: N—((S)-1-((3P)-3-(4-chloro-3-(methylsulfonamido)-1-(2,2,2-trifluoroethyl)-1H-indazol-7-yl)-7-(2-fluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

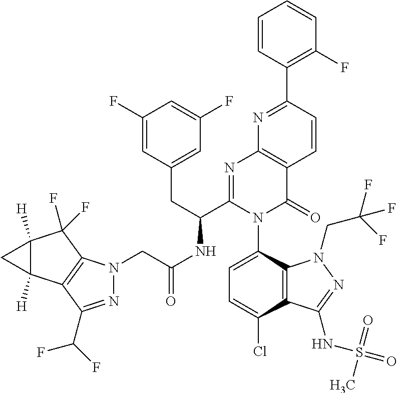

To a stirred solution of (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-(2,2,2-trifluoroethyl)-1H-indazol-3-yl)methanesulfonamide (0.04 g, 0.055 mmol) in N,N-Dimethylformamide (1 mL) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.015 g, 0.055 mmol), 2-(3H-[1,2,3]triazolo[4,5-b]pyridin-3-yl)-1,1,3,3-tetramethylisouronium hexafluorophosphate(V) (“HATU”, 0.025 g, 0.066 mmol) and DIPEA (0.024 mL, 0.138 mmol). The reaction mixture was stirred at room temperature for 1 h and then was diluted with 1 mL of DMF, the solution was filtered and the filtrate was subjected to prep-HPLC purification to afford the title compound, N—((S)-1-((3P)-3-(4-chloro-3-(methylsulfonamido)-1-(2,2,2-trifluoroethyl)-1H-indazol-7-yl)-7-(2-fluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,a,5-tetrahydro-1H-cyclopropa[3,4]cyclopenta[1,2-c]pyrazol-1-yl)acetamide.

The sample was analyzed using LCMS Method F: retention time=1.47 min.; observed ion=968.2 (M+H). 1H NMR (500 MHz, METHANOL-d4) δ ppm 8.73-8.78 (m, 1H) 8.09-8.17 (m, 2H) 7.60-7.68 (m, 1H) 7.34-7.51 (m, 4H) 6.47-6.85 (m, 4H) 4.65-4.84 (m, 4H) 4.25-4.36 (m, 1H) 3.37-3.42 (m, 1H) 3.25-3.27 (m, 3H) 3.05-3.12 (m, 1H) 2.39-2.50 (m, 2H) 1.35-1.42 (m, 1H) 0.99-1.04 (m, 1H)

### Preparation of Example 28: N—((S)-1-((3P)-3-(4-chloro-1-methyl-3-(methylsulfonamido)-1H-indazol-7-yl)-4-oxo-7-(2,4,6-trifluorophenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

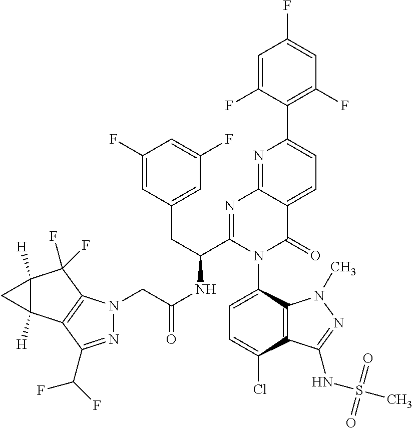

The title compound was prepared according to General Procedure A using (2,4,6-trifluorophenyl)boronic acid as the coupling partner. The experiment afforded the title compound, N—((S)-1-((3P)-3-(4-chloro-1-methyl-3-(methylsulfonamido)-1H-indazol-7-yl)-4-oxo-7-(2,4,6-trifluorophenyl)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.45 min.; observed ion=936.3 (M+H). 1H NMR (500 MHz, METHANOL-d4) δ ppm 8.73 (d, J=8.05 Hz, 1H) 7.72-7.81 (m, 1H) 7.15-7.29 (m, 2H) 6.98-7.10 (m, 2H) 6.42-6.75 (m, 4H) 4.36-4.54 (m, 2H) 3.54-3.59 (m, 3H) 3.33-3.43 (m, 1H) 3.15-3.16 (m, 3H) 3.01-3.06 (m, 1H) 2.27-2.34 (m, 2H) 1.22-1.27 (m, 1H) 0.85-0.91 (m, 1H)

### Preparation of Example 29: N—((S)-1-((3P)-3-(4-chloro-1-methyl-3-(methylsulfonamido)-1H-indazol-7-yl)-7-(3-chloro-4-fluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

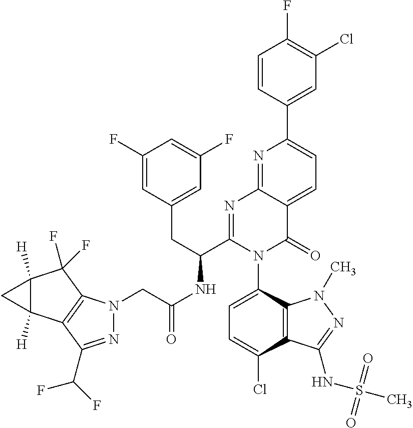

The title compound was prepared according to General Procedure A using (3-chloro-4-fluorophenyl)boronic acid as the coupling partner. The experiment afforded the title compound, N—((S)-1-((3P)-3-(4-chloro-1-methyl-3-(methylsulfonamido)-1H-indazol-7-yl)-7-(3-chloro-4-fluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.6 min.; observed ion=934.3 (M+H). 1H NMR (500 MHz, METHANOL-d4) δ ppm 8.64-8.72 (m, 1H) 8.56-8.63 (m, 1H) 8.43-8.52 (m, 1H) 8.12-8.19 (m, 1H) 7.44-7.53 (m, 1H) 7.15-7.25 (m, 2H) 6.39-6.73 (m, 4H) 4.40-4.56 (m, 2H) 3.52-3.58 (m, 3H) 3.36-3.44 (m, 1H) 3.13-3.16 (m, 3H) 3.03-3.09 (m, 1H) 2.26-2.35 (m, 2H) 1.21-1.27 (m, 1H) 0.85-0.92 (m, 1H)

### Preparation of Example 30: N—((S)-1-((3P)-3-(4-chloro-1-methyl-3-(methylsulfonamido)-1H-indazol-7-yl)-7-(3-cyano-4-fluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

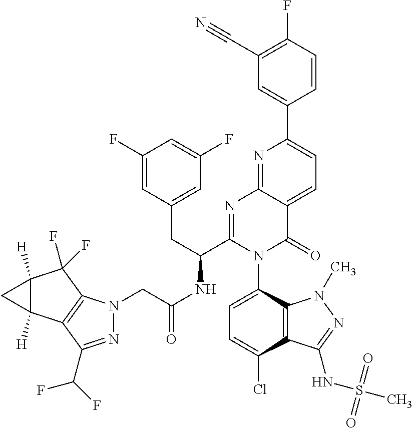

The title compound was prepared according to General Procedure A using (3-cyano-4-fluorophenyl)boronic acid as the coupling partner. The experiment afforded the title compound, N—((S)-1-((3P)-3-(4-chloro-1-methyl-3-(methylsulfonamido)-1H-indazol-7-yl)-7-(3-cyano-4-fluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.5 min.; observed ion=925.4 (M+H). 1H NMR (500 MHz, METHANOL-d4) δ ppm 8.72-8.84 (m, 2H) 8.65 (ddd, J=8.94, 5.07, 2.38 Hz, 1H) 8.21-8.27 (m, 1H) 7.62 (t, J=8.94 Hz, 1H) 7.23-7.35 (m, 2H) 6.51-6.82 (m, 4H) 4.54-4.65 (m, 2H) 3.63 (s, 3H) 3.50 (dd, J=14.01, 4.47 Hz, 1H) 3.22 (s, 3H) 3.17 (s, 1H) 2.36-2.45 (m, 2H) 1.31-1.37 (m, 1H) 0.96-1.04 (m, 1H)

### Preparation of Example 31: N—((S)-1-((3P)-3-(4-chloro-1-methyl-3-(methylsulfonamido)-1H-indazol-7-yl)-6-(2-fluorophenyl)-7-methoxy-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide

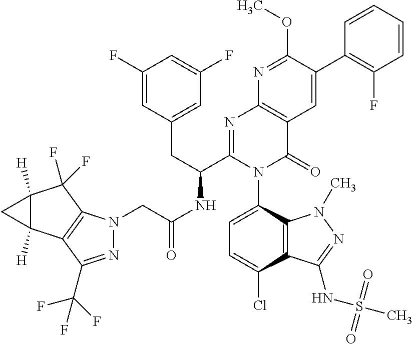

To a stirred solution of (S)—N-((6P)-7-(2-(1-amino-2-(3,5-difluorophenyl)ethyl)-6-(2-fluorophenyl)-7-methoxy-4-oxopyrido[2,3-d]pyrimidin-3(4H)-yl)-4-chloro-1-methyl-1H-indazol-3-yl)methanesulfonamide (0.036 g, 0.053 mmol) in N,N-Dimethylformamide (1 mL) was added 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid (0.015 g, 0.053 mmol), 2-(3H-[1,2,3]triazolo[4,5-b]pyridin-3-yl)-1,1,3,3-tetramethylisouronium hexafluorophosphate(V) (“HATU”, 0.024 g, 0.064 mmol) and DIPEA (0.023 mL, 0.133 mmol). The reaction mixture was stirred at room temperature for 1 h and then was diluted with DMF (1 mL), the solution was filtered and the filtrate was subjected to prep-HPLC purification to afford the title compound, N—((S)-1-((3P)-3-(4-chloro-1-methyl-3-(methylsulfonamido)-1H-indazol-7-yl)-6-(2-fluorophenyl)-7-methoxy-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide. The sample was analyzed using LCMS Method F: retention time=1.5 min.; observed ion=948.3 (M+H). 1H NMR (500 MHz, METHANOL-d4) δ ppm 8.38-8.43 (m, 1H) 7.43-7.51 (m, 2H) 7.18-7.32 (m, 4H) 6.74-6.82 (m, 1H) 6.57-6.64 (m, 2H) 4.54-4.71 (m, 3H) 4.12-4.17 (m, 3H) 3.60-3.65 (m, 3H) 3.45-3.52 (m, 1H) 3.21-3.25 (m, 3H) 3.10-3.17 (m, 1H) 2.39-2.52 (m, 2H) 1.35-1.42 (m, 1H) 1.02-1.08 (m, 1H)

### Preparation of Example 32: N—((S)-1-((3P)-3-(4-chloro-1-(2,2-difluoroethyl)-3-(methylsulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide

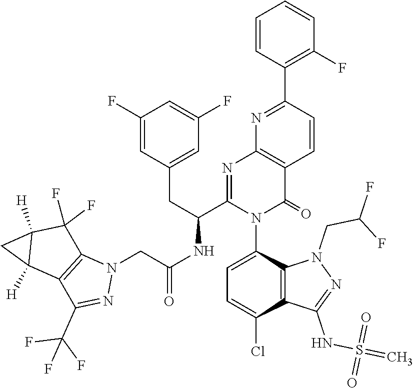

To a stirred solution of (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-(2,2-difluoroethyl)-1H-indazol-3-yl)methanesulfonamide (0.037 g, 0.053 mmol) in N,N-Dimethylformamide (1 mL) was added 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid (0.015 g, 0.053 mmol), 2-(3H-[1,2,3]triazolo[4,5-b]pyridin-3-yl)-1,1,3,3-tetramethylisouronium hexafluorophosphate(V) (“HATU”, 0.024 g, 0.064 mmol) and DIPEA (0.023 mL, 0.133 mmol). The reaction mixture was stirred at room temperature for 1 h and then was diluted with DMF (1 mL), the solution was filtered, and the filtrate was subjected to prep-HPLC purification to afford the title compound, N—((S)-1-((3P)-3-(4-chloro-1-(2,2-difluoroethyl)-3-(methylsulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide. The sample was analyzed using LCMS Method F: retention time=1.47 min.; observed ion=968.4 (M+H). 1H NMR (500 MHz, METHANOL-d4) δ ppm 8.70-8.79 (m, 1H) 8.05-8.14 (m, 2H) 7.57-7.66 (m, 1H) 7.32-7.46 (m, 4H) 6.73-6.82 (m, 1H) 6.52-6.59 (m, 2H) 5.87-6.15 (m, 1H) 4.63-4.79 (m, 3H) 4.33-4.45 (m, 1H) 3.93-4.06 (m, 1H) 3.40-3.47 (m, 1H) 3.24-3.26 (m, 3H) 3.08-3.14 (m, 1H) 2.33-2.50 (m, 2H) 1.31-1.41 (m, 1H) 0.99-1.07 (m, 1H)

### Preparation of Example 33: (S)—N-(1-((3P)-3-(4-chloro-1-(2,2-difluoroethyl)-3-(methylsulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)-2-(3-cyclopropyl-1H-pyrazol-1-yl)acetamide

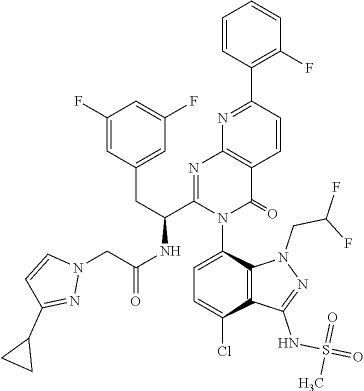

To a stirred solution of (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-(2,2-difluoroethyl)-1H-indazol-3-yl)methanesulfonamide (0.04 g, 0.057 mmol) in N,N-Dimethylformamide (1 mL) was added 2-(3-cyclopropyl-1H-pyrazol-1-yl)acetic acid (9.44 mg, 0.057 mmol), 2-(3H-[1,2,3]triazolo[4,5-b]pyridin-3-yl)-1,1,3,3-tetramethylisouronium hexafluorophosphate(V) (“HATU”, 0.026 g, 0.068 mmol) and DIPEA (0.025 mL, 0.142 mmol). The reaction mixture was stirred at room temperature for 1 h and then was diluted with DMF (1 mL), the solution was filtered, and the filtrate was subjected to prep-HPLC purification to afford the title compound, (S)—N-(1-((3P)-3-(4-chloro-1-(2,2-difluoroethyl)-3-(methylsulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)-2-(3-cyclopropyl-1H-pyrazol-1-yl)acetamide. The sample was analyzed using LCMS Method F: retention time=1.41 min.; observed ion=852.4 (M+H). 1H NMR (500 MHz, METHANOL-d4) δ ppm 8.73-8.82 (m, 1H) 8.08-8.18 (m, 2H) 7.60-7.68 (m, 1H) 7.33-7.50 (m, 5H) 6.75-6.82 (m, 1H) 6.57-6.67 (m, 2H) 5.89-6.18 (m, 2H) 4.83-4.85 (m, 1H) 4.48-4.60 (m, 2H) 4.33-4.45 (m, 1H) 3.95-4.09 (m, 1H) 3.44-3.51 (m, 1H) 3.24-3.28 (m, 3H) 3.08-3.17 (m, 1H) 1.80-1.89 (m, 1H) 0.81-0.89 (m, 2H) 0.61-0.68 (m, 2H)

### Preparation of Example 34: (S)—N-(1-((3P)-3-(4-chloro-1-methyl-3-(methylsulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)-2-(3-cyclopropyl-1H-pyrazol-1-yl)acetamide

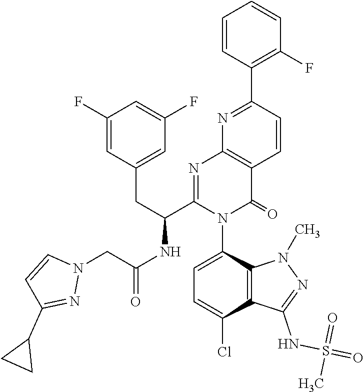

To a stirred solution of (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-methyl-1H-indazol-3-yl)methanesulfonamide (0.04 g, 0.061 mmol) in N,N-Dimethylformamide (1 mL) was added 2-(3-cyclopropyl-1H-pyrazol-1-yl)acetic acid (10.16 mg, 0.061 mmol), 2-(3H-[1,2,3]triazolo[4,5-b]pyridin-3-yl)-1,1,3,3-tetramethylisouronium pentafluoro(methyl)phosphate(V) (“HATU”, 0.028 g, 0.073 mmol) and DIPEA (0.027 mL, 0.153 mmol). The reaction mixture was stirred at room temperature for 1 h and then was diluted with DMF (1 mL), the solution was filtered, and the filtrate was subjected to prep-HPLC purification to afford the title compound, (S)—N-(1-((3P)-3-(4-chloro-1-methyl-3-(methylsulfonamido)-1H-indazol-7-yl)-7-(2-fluorophenyl)-4-oxo-3,4-dihydropyrido[2,3-d]pyrimidin-2-yl)-2-(3,5-difluorophenyl)ethyl)-2-(3-cyclopropyl-1H-pyrazol-1-yl)acetamide. The sample was analyzed using LCMS Method F: retention time=1.41 min.; observed ion=802.4 (M+H). 1H NMR (500 MHz, METHANOL-d4) δ ppm 8.75-8.82 (m, 1H) 8.08-8.16 (m, 2H) 7.59-7.68 (m, 1H) 7.29-7.48 (m, 5H) 6.65-6.81 (m, 3H) 5.89-5.93 (m, 1H) 4.96-5.04 (m, 1H) 4.32-4.49 (m, 2H) 3.64-3.69 (m, 3H) 3.52-3.59 (m, 1H) 3.27-3.29 (m, 3H) 3.13-3.19 (m, 1H) 1.81-1.89 (m, 1H) 0.82-0.89 (m, 2H) 0.61-0.67 (m, 2H)

### Preparation of Example 35: (S)—N-(1-((3P)-3-(4-chloro-3-(cyclopropanesulfonamido)-1-(2,2-difluoroethyl)-1H-indazol-7-yl)-7-(2-fluorophenyl)-4-oxo-3,4-dihydropyrido[2,3-d]pyrimidin-2-yl)-2-(3,5-difluorophenyl)ethyl)-2-(3-cyclopropyl-1H-pyrazol-1-yl)acetamide

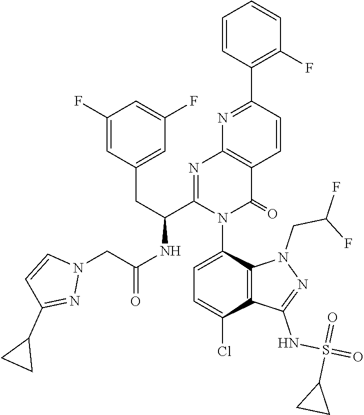

To a stirred solution of (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-(2,2-difluoroethyl)-1H-indazol-3-yl)-N-(4-methoxybenzyl)cyclopropanesulfonamide (0.04 g, 0.047 mmol) in N,N-Dimethylformamide (1 mL) was added 2-(3-cyclopropyl-1H-pyrazol-1-yl)acetic acid (7.82 mg, 0.047 mmol), 2-(3H-[1,2,3]triazolo[4,5-b]pyridin-3-yl)-1,1,3,3-tetramethylisouronium hexafluorophosphate(V) (“HATU”, 0.021 g, 0.056 mmol) and DIPEA (0.021 mL, 0.118 mmol). The reaction mixture was stirred for 18 h and then was concentrated in vacuo. The residue was dissolved in DCM/TFA (1:1, 2 mL) and to the solution was added triflic acid (0.017 mL, 0.188 mmol). The purple solution was stirred for 1 h and then was concentrated under reduced pressure. The residue was taken up in DCM (1.5 mL). The solution was washed with sat. aq. NaHCO₃(1 mL) and then was concentrated under reduced pressure. The residue was dissolved in DMF (2 mL), the solution was filtered, and the filtrate was subjected to prep-HPLC purification to afford the title compound, (S)—N-(1-((3P)-3-(4-chloro-3-(cyclopropanesulfonamido)-1-(2,2-difluoroethyl)-1H-indazol-7-yl)-7-(2-fluorophenyl)-4-oxo-3,4-dihydropyrido[2,3-d]pyrimidin-2-yl)-2-(3,5-difluorophenyl)ethyl)-2-(3-cyclopropyl-1H-pyrazol-1-yl)acetamide. The sample was analyzed using LCMS Method F: retention time=1.45 min.; observed ion=878.5 (M+H). 1H NMR (500 MHz, METHANOL-d4) δ ppm 8.73-8.82 (m, 1H) 8.09-8.20 (m, 2H) 7.60-7.68 (m, 1H) 7.32-7.50 (m, 5H) 6.75-6.85 (m, 1H) 6.56-6.65 (m, 2H) 5.87-6.19 (m, 2H) 4.83-4.87 (m, 1H) 4.50-4.60 (m, 2H) 4.34-4.46 (m, 1H) 3.93-4.10 (m, 1H) 3.43-3.51 (m, 1H) 3.10-3.17 (m, 1H) 2.92-3.01 (m, 1H) 1.80-1.89 (m, 1H) 1.10-1.19 (m, 2H) 1.00-1.05 (m, 2H) 0.83-0.89 (m, 2H) 0.61-0.68 (m, 2H)

### Preparation of Example 36: (S)—N-(1-((3P)-3-(4-chloro-3-(methylsulfonamido)-1-(2,2,2-trifluoroethyl)-1H-indazol-7-yl)-7-(2-fluorophenyl)-4-oxo-3,4-dihydropyrido[2,3-d]pyrimidin-2-yl)-2-(3,5-difluorophenyl)ethyl)-2-(3-cyclopropyl-1H-pyrazol-1-yl)acetamide

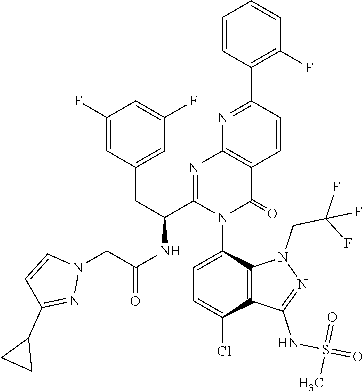

To a stirred solution of (S)—N-((6P)-7-(2-(1-amino-2-(3,5-difluorophenyl)ethyl)-7-(2-fluorophenyl)-4-oxopyrido[2,3-d]pyrimidin-3(4H)-yl)-4-chloro-1-(2,2,2-trifluoroethyl)-1H-indazol-3-yl)methanesulfonamide (0.04 g, 0.055 mmol) in N,N-Dimethylformamide (DMF) (1 mL) was added 2-(3-cyclopropyl-1H-pyrazol-1-yl)acetic acid (9.21 mg, 0.055 mmol), 2-(3H-[1,2,3]triazolo[4,5-b]pyridin-3-yl)-1,1,3,3-tetramethylisouronium hexafluorophosphate(V) (“HATU”, 0.025 g, 0.066 mmol) and DIPEA (0.024 mL, 0.138 mmol). The reaction mixture was stirred at room temperature for 1 h and then was diluted with DMF (1 mL), the solution was filtered, and the filtrate was subjected to prep-HPLC purification to afford the title compound, (S)—N-(1-((3P)-3-(4-chloro-3-(methylsulfonamido)-1-(2,2,2-trifluoroethyl)-1H-indazol-7-yl)-7-(2-fluorophenyl)-4-oxo-3,4-dihydropyrido[2,3-d]pyrimidin-2-yl)-2-(3,5-difluorophenyl)ethyl)-2-(3-cyclopropyl-1H-pyrazol-1-yl)acetamide. The sample was analyzed using LCMS Method F: retention time=1.43 min.; observed ion=870.1 (M+H). 1H NMR (500 MHz, METHANOL-d4) δ ppm 8.70-8.81 (m, 1H) 8.07-8.19 (m, 2H) 7.61-7.69 (m, 1H) 7.34-7.49 (m, 5H) 6.74-6.83 (m, 1H) 6.50-6.58 (m, 2H) 5.90-5.97 (m, 1H) 4.73-4.82 (m, 3H) 4.25-4.37 (m, 1H) 3.38-3.45 (m, 1H) 3.28 (s, 3H) 3.06-3.13 (m, 1H) 1.82-1.89 (m, 1H) 1.32-1.39 (m, 1H) 0.82-0.90 (m, 2H) 0.61-0.68 (m, 2H)

### Preparation of Example 37: N—((S)-1-((3P)-3-(4-chloro-1-methyl-3-(methylsulfonamido)-1H-indazol-7-yl)-7-(2,4-difluorophenyl)-4-oxo-3,4-dihydropyrido[3,2-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

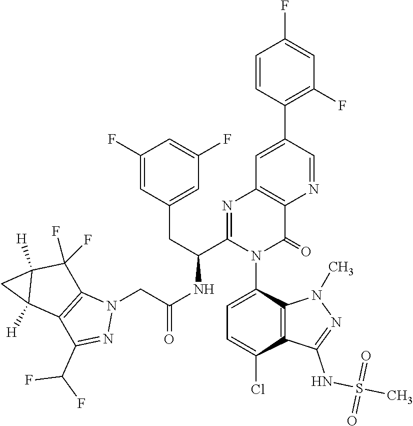

N—((S)-1-((3P)-7-bromo-3-(4-chloro-1-methyl-3-(methylsulfonamido)-1H-indazol-7-yl)-4-oxo-3,4-dihydropyrido[3,2-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (0.05 g, 0.056 mmol), tripotassium phosphate (0.036 g, 0.169 mmol), dichloro[9,9-dimethyl-4,5-bis(diphenylphosphino)xanthene]palladium(II) (2.135 mg, 2.82 μmol) and (2,4-difluorophenyl)boronic acid (0.027 g, 0.169 mmol) were combined dry in a 1 dram vial equipped with a stir bar. The vial was purged with Ar and then to the vial was added tetrahydrofuran (1 ml) and water (0.250 mL). The mixture was stirred at rt overnight. The mixture was concentrated under reduced pressure and the residue was dissolved in DMF (2 mL), the solution was filtered, and the filtrate was subjected to prep-HPLC purification to afford the title compound, N—((S)-1-((3P)-3-(4-chloro-1-methyl-3-(methylsulfonamido)-1H-indazol-7-yl)-7-(2,4-difluorophenyl)-4-oxo-3,4-dihydropyrido[3,2-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.41 min.; observed ion=918.3 (M+H). 1H NMR (500 MHz, METHANOL-d4) δ ppm 9.05-9.19 (m, 1H) 8.45-8.54 (m, 1H) 7.74-7.83 (m, 1H) 7.56-7.65 (m, 1H) 7.44-7.48 (m, 1H) 7.38-7.43 (m, 1H) 7.36-7.38 (m, 1H) 6.56-6.84 (m, 4H) 4.85-4.87 (m, 1H) 4.44-4.59 (m, 2H) 3.64-3.70 (m, 3H) 3.47-3.53 (m, 1H) 3.26-3.27 (m, 3H) 3.12-3.18 (m, 1H) 2.39-2.46 (m, 2H) 1.33-1.39 (m, 1H) 0.97-1.02 (m, 1H

### Preparation of Example 38: N—((S)-1-((3P)-3-(4-chloro-1-methyl-3-(methylsulfonamido)-1H-indazol-7-yl)-7-(2-fluorophenyl)-4-oxo-3,4-dihydropyrido[3,2-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

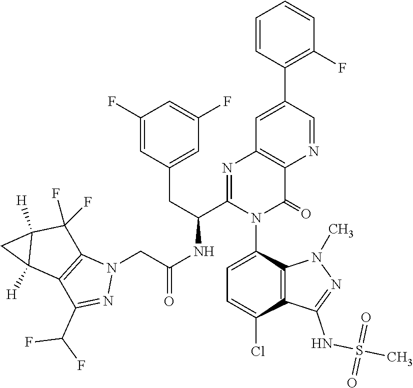

N—((S)-1-((3P)-7-bromo-3-(4-chloro-1-methyl-3-(methylsulfonamido)-1H-indazol-7-yl)-4-oxo-3,4-dihydropyrido[3,2-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (0.05 g, 0.056 mmol), tripotassium phosphate (0.036 g, 0.169 mmol), dichloro[9,9-dimethyl-4,5-bis(diphenylphosphino)xanthene]palladium(II) (2.135 mg, 2.82 μmol) and (2-fluorophenyl)boronic acid (0.024 g, 0.169 mmol) were combined in a dry 1 dram vial equipped with a stir bar. The vial was purged with Ar and then to the vial was added tetrahydrofuran (1 mL) and water (0.250 mL). The reaction mixture was stirred at rt overnight. The mixture was concentrated under reduced pressure and the residue was dissolved in DMF (2 mL), the solution was filtered, and the filtrate was subjected to prep-HPLC purification to afford the title compound, N—((S)-1-((3P)-3-(4-chloro-1-methyl-3-(methylsulfonamido)-1H-indazol-7-yl)-7-(2-fluorophenyl)-4-oxo-3,4-dihydropyrido[3,2-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.39 min.; observed ion=900.3 (M+H). 1H NMR (500 MHz, METHANOL-d4) δ ppm 9.06-9.12 (m, 1H) 8.47-8.53 (m, 1H) 7.74-7.82 (m, 1H) 7.57-7.65 (m, 1H) 7.32-7.49 (m, 4H) 6.55-6.83 (m, 4H) 4.85-4.87 (m, 1H) 4.45-4.59 (m, 2H) 3.64-3.71 (m, 3H) 3.47-3.54 (m, 1H) 3.26-3.27 (m, 3H) 3.11-3.18 (m, 1H) 2.39-2.46 (m, 2H) 1.34-1.39 (m, 1H) 0.97-1.02 (m, 1H)

### Preparation of Example 39: N—((S)-1-((3P)-3-(4-chloro-1-methyl-3-(methylsulfonamido)-1H-indazol-7-yl)-7-(2,4-difluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

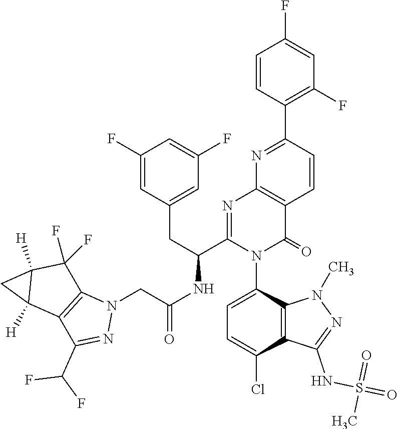

To a stirred solution of (S)—N-((6P)-7-(2-(1-amino-2-(3,5-difluorophenyl)ethyl)-7-(2,4-difluorophenyl)-4-oxopyrido[2,3-d]pyrimidin-3(4H)-yl)-4-chloro-1-methyl-1H-indazol-3-yl)-N-(4-methoxybenzyl)methanesulfonamide (0.05 g, 0.063 mmol) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.017 g, 0.063 mmol), 2-(3H-[1,2,3]triazolo[4,5-b]pyridin-3-yl)-1,1,3,3-tetramethylisouronium hexafluorophosphate(V) (“HATU”, 0.029 g, 0.076 mmol) and DIPEA (0.028 ml, 0.158 mmol). The reaction mixture was stirred at rt for 18 h. The solution was concentrated under reduced pressure and the residue was dissolved in DCM/TFA (1:1, 2 mL). To the solution was added triflic acid (0.017 mL, 0.188 mmol) and the resulting purple solution was stirred for 1 h. The solution was concentrated and the residue was dissolved in DCM (1.5 mL). The solution was washed with sat. aq. NaHCO₃(1 mL) and then was concentrated under reduced pressure. The residue was dissolved in DMF (2 mL), the solution was filtered, and the filtrate was subjected to prep-HPLC purification to afford the title compound, N—((S)-1-((3P)-3-(4-chloro-1-methyl-3-(methylsulfonamido)-1H-indazol-7-yl)-7-(2,4-difluorophenyl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.48 min.; observed ion=918.2 (M+H). 1H NMR (500 MHz, METHANOL-d4) δ ppm 8.62-8.71 (m, 1H) 7.91-8.13 (m, 2H) 7.10-7.25 (m, 4H) 6.42-6.73 (m, 4H) 4.38-4.53 (m, 2H) 3.49-3.57 (m, 3H) 3.36-3.43 (m, 1H) 3.13-3.15 (m, 3H) 3.01-3.06 (m, 1H) 2.26-2.34 (m, 2H) 1.22-1.26 (m, 1H) 0.86-0.91 (m, 1H)

### IUPAC Chemical Names:

The IUPAC chemical names for each example are listed below. At this time these names are not recognized by common software such tools such as ChemDraw or JChem. Therefore, the chemical names used throughout the Examples section above were generated with ChemDraw and the chemical names can be converted to chemical structures using ChemDraw after the P/M nomenclature—e.g., “(3P)-”—is removed.

| Example | IUPAC Name |
|---|---|
| Example 1 | N-[(1S)-1-[(3P)-3-(4-chloro-3-methanesulfonamido-1-methyl-1H-indazol-7-yl)-7-(2-fluorophenyl)- |
| | 4-oxo-3H,4H-pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8-dien-7-yl]acetamide |
| Example 2 | N-[(1S)-1-[(3P)-3-(4-chloro-3-methanesulfonamido-1-methyl-1H-indazol-7-yl)-6-(2-fluorophenyl)- |
| | 7-methoxy-4-oxo-3H,4H-pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8-dien-7-yl]acetamide |
| Example 3 | N-[(1S)-1-[(3P)-3-(4-chloro-3-methanesulfonamido-1-methyl-1H-indazol-7-yl)-7-(2,6- |
| | difluorophenyl)-4-oxo-3H,4H-pyrido[2,3-d]pyrimidin-2-yl]-2-(3,5-difluorophenyl)ethyl]-2-[(2S,4R)- |
| | 9-(difluoromethyl)-5,5-difluoro-7,8-diazatricyclo[4.3.0.0²,⁴]nona-1(6),8-dien-7-yl]acetamide |
| Example 4 | N-[(1S)-1-[(3P)-3-(4-chloro-3-methanesulfonamido-1-methyl-1H-indazol-7-yl)-7-(2,3- |
| | difluorophenyl)-4-oxo-3H,4H-pyrido[2,3-d]pyrimidin-2-yl]-2-(3,5-difluorophenyl)ethyl]-2-[(2S,4R)- |
| | 9-(difluoromethyl)-5,5-difluoro-7,8-diazatricyclo[4.3.0.0²,⁴]nona-1(6),8-dien-7-yl]acetamide |
| Example 5 | N-[(1S)-1-[(3P)-3-(4-chloro-3-methanesulfonamido-1-methyl-1H-indazol-7-yl)-7-[4-fluoro-3- |
| | (hydroxymethyl)phenyl]-4-oxo-3H,4H-pyrido[2,3-d]pyrimidin-2-yl]-2-(3,5-difluorophenyl)ethyl]-2- |
| | [(2S,4R)-9-(difluoromethyl)-5,5-difluoro-7,8-diazatricyclo[4.3.0.0²,⁴]nona-1(6),8-dien-7-yl]acetamide |
| Example 6 | N-[(1S)-1-[(3P)-7-(5-chloro-2,4-difluorophenyl)-3-(4-chloro-3-methanesulfonamido-1-methyl-1H- |
| | indazol-7-yl)-4-oxo-3H,4H-pyrido[2,3-d]pyrimidin-2-yl-2-(3,5-difluorophenyl)ethyl]-2-[(2S,4R)- |
| | 9-(difluoromethyl)-5,5-difluoro-7,8-diazatricyclo[4.3.0.0²,⁴]nona-1(6),8-dien-7-yl]acetamide |
| Example 7 | N-[(1S)-1-[(3P)-3-[4-chloro-3-cyclopropanesulfonamido-1-(2,2-difluoroethyl)-1H-indazol-7-yl]-7- |
| | (2-flurophenyl)-4-oxo-3H,4H-pyrido[2,3-d]pyrimidin-2-yl]-2-(3,5-difluorophenyl)ethyl]-2-[(2S,4R)- |
| | 5,5-difluoro-9-(trifluoromethyl)-7,8-diazatricyclo[4.3.0.0²,⁴]nona-1(6),8-dien-7-yl]acetamide |
| Example 8 | N-[(1S)-1-[(3P)-7-(4-chloro-2-fluorophenyl)-3-(4-chloro-3-methanesulfonamido-1-methyl-1H- |
| | indazol-7-yl)-4-oxo-3H,4H-pyrido[2,3-d]pyrimidin-2-yl]-2-(3,5-difluorophenyl)ethyl]-2-[(2S,4R)- |
| | 9-(difluoromethyl-5,5-difluoro-7,8-diazatricyclo[4.3.0.0²,⁴]nona-1(6),8-dien-7-yl]acetamide |
| Example 9 | N-[(1S)-1-[(3P)-3-(4-chloro-3-methanesulfonamido-1-methyl-1H-indazol-7-yl)-7-(2,5-difluorophenyl)- |
| | 4-oxo-3H,4H-pyrido[2,3-d]pyrimidin-2-yl]-2-(3,5-difluorophenyl)ethyl-2-[(2S,4R)-9-(difluoromethyl)- |
| | 5,5-difluoro-7,8-diazatricyclo[4.3.0.0²,⁴]nona-1(6),8-dien-7-yl]acetamide |
| Example 10 | N-[(1S)-1-[(3P)-3-[4-chloro-1-(2,2-difluoroethyl)-3-methanesulfonamido-1H-indazol-7-yl]-7- |
| | (2-fluorophenyl)-4-oxo-3H,4H-pyrido[2,3-d]pyrimidin-2-yl]-2-(3,5-difluorophenyl)ethyl]-2- |
| | [(2S,4R)-9-(difluoromethyl)-5,5-difluoro-7,8-diazatricyclo[4.3.0.0²,⁴]none-1(6),8-dien-7-yl]acetamide |
| Example 11 | N-[(1S)-1-[(3P)-3-[4-chloro-3-methanesulfonamido-1-(2,2,2-trifluoroethyl)-1H-indazol-7-yl]-7- |
| | (2-fluorophenyl)-4-oxo-3H,4H-pyrido[2,3-d]pyrimidin-2-yl]-2-(3,5-difluorophenyl)ethyl]-2-[(2S,4R)- |
| | 5,5-difluoro-9-(trifluoromethyl)-7,8-diazatricyclo[4.3.0.0²,⁴]nona-1(6),8-dien-7-yl]acetamide |
| Example 12 | N-[(1S)-1-[(3P)-3-(4-chloro-3-methanesulfonamido-1-methyl-1H-indazol-7-yl)-4-oxo-7-(3,4,5- |
| | trifluorophenyl)-3H,4H-pyrido[2,3-d]pyrimidin-2-yl]-2-(3,5-difluorophenyl)ethyl]-2-[(2S,4R)- |
| | 9-(difluoromethyl)-5,5-difluoro-7,8-diazatricyclo[4.3.0.0²,⁴]nona-1(6),8-dien-7-yl]acetamide |
| Example 13 | N-[(1S)-1-[(3P)-3-(4-chloro-3-methanesulfonamido-1-methyl-1H-indazole-7-yl)-7-(4-chlorophenyl)-4- |
| | oxo-3H,4H-pyrido[2,3-d]pyrimidin-2-yl]-2-(3,5-difluorophenyl)ethyl]-2-[(2S,4R)-9-(difluoromethyl)- |
| | 5,5-difluoro-7,8-diazatricyclo[4.3.0.0²,⁴]nona-1(6),8-dien-7-yl]acetamide |
| Example 14 | N-[(1S)-1-[(3P)-3-(4-chloro-3-methanesulfonamido-1-methyl-1H-indazol-7-yl)-4-oxo-7-[4- |
| | (trifluoromethyl)phenyl]-3H,4H-pyrido[2,3-d]pyrimidin-2-yl]-2-(3,5-difluorophenyl)ethyl]-2-[(2S,4R)- |
| | 9-(difluoromethyl)-5,5-difluoro-7,8-diazatricyclo[4.3.0.0²,⁴]nona-1(6),8-dien-7-yl]acetamide |
| Example 15 | N-[(1S)-1-[(3P)-3-(4-chloro-3-methanesulfonamido-1-methyl-1H-indazol-7-yl)-4-oxo-7-(2,3,4- |
| | trifluorophenyl)-3H,4H-pyrido[2,3-d]pyrimidin-2-yl]-2-(3,5-difluorophenyl)ethyl]-2-[(2S,4R)- |
| | 9-(difluoromethyl)-5,5-difluoro-7,8-diazatricyclo[4.3.0.0²,⁴]nona-1(6),8-diene-7-yl]acetamide |
| Example 16 | N-[(1S)-1-[(3P)-3-(4-chloro-3-methanesulfonamido-1-methyl-1H-indazol-7-yl)-7-[4-fluoro-2- |
| | (hydroxymethyl)phenyl]-4-oxo-3H,4H-pyrido[2,3-d]pyrimidin-2-yl]-2-(3,5-difluorophenyl)ethyl]-2- |
| | [(2S,4R)-9-(difluoromethyl)-5,5-difluoro-7,8-diazatricyclo[4.3.0.0²,⁴]nona-1(6),8-dien-7-yl]acetamide |
| Example 17 | N-[(1S)-1-[(3P)-3-(4-chloro-3-methanesulfonamido-1-methyl-1H-indazole-7-yl)-7-(2-fluorophenyl)- |
| | 4-oxo-3H,4H-pyrido[2,3-d]pyrimidin-2-yl]-2-(3,5-difluorophenyl)ethyl]-2-[(2S,4R)-5,5-difluoro-9- |
| | (trifluoromethyl)-7,8-diazatricyclo[4.3.0.0²,⁴]nona-1(6),8-dien-7-yl]acetamide |
| Example 18 | N-[(1S)-1-[(3P)-3-(4-chloro-3-methanesulfonamido-1-methyl-1H-indazol-7-yl)-7-(4-fluorophenyl)- |
| | 4-oxo-3H,4H-pyrido[2,3-d]pyrimidin-2-yl]-2-(3,5-difluorophenyl)ethyl]-2-[(2S,4R)-9-(difluoromethyl)- |
| | 5,5-difluoro-7,8-diazatricyclo[4.3.0.0²,⁴]nona-1(6),8-dien-7-yl]acetamide |
| Example 19 | N-[(1S)-1-[(3P)-3-(4-chloro-3-methanesulfonamido-1-methyl-1H-indazol-7-yl)-7-(4-fluoro-2- |
| | methanesulfonylphenyl)-4-oxo-3H,4H-pyrido[2,3-d]pyrimidin-2-yl]-2-(3,5-difluorophenyl)ethyl]- |
| | 2-[(2S,4R)-9-(difluoromethyl)-5,5-difluoro-7,8-diazatricyclo[4.3.0.0²,⁴]nona-1(6),8-dien-7-yl]acetamide |
| Example 20 | N-[(1S)-1-[(3P)-3-[4-chloro-3-cyclopropanesulfonamido-1-(2,2-difluoroethyl)-1H-indazol-7-yl]-7- |
| | (2-fluorophenyl)-4-oxo-3H,4H-pyrido[2,3-d]pyrimidin-2-yl]-2-(3,5-difluorophenyl)ethyl]-2-[(2S,4R)- |
| | 9-(difluoromethyl)-5,5-difluoro-7,8-diazatricyclo[4.3.0.0²,⁴]nona-1(6),8-diene-7-yl]acetamide |
| Example 21 | N-[(1S)-1-[(3P)-3-(4-chloro-3-methanesulfonamido-1-methyl-1H-indazol-7-yl)-7-yl)-7-(3,5- |
| | difluorophenyl)-4-oxo-3H,4H-pyrido[2,3-d]pyrimidin-2-yl]-2-(3,5-difluorophenyl)ethyl]-2-[(2S,4R)- |
| | 9-(difluoromethyl)-5,5-difluoro-7,8-diazatricyclo[4.3.0.0²,⁴]nona-1(6),8-dien-7-yl]acetamide |
| Example 22 | N-[(1S)-1-[(3P)-7-(3-chloro-2,4-difluorophenyl)-3-(4-chloro-3-methanesulfonamido-1-methyl-1H- |
| | indazol-7-yl)-4-oxo-3H,4H-pyrido[2,3-d]pyrimidin-2-yl]-2-(3,5-difluorophenyl)ethyl]-2-[(2S,4R)- |
| | 9-(difluoromethyl)-5,5-difluoro-7,8-diazatricyclo[4.3.0.0²,⁴]nona-1(6),8-dien-7-yl]acetamide |
| Example 23 | N-[(1S)-1-[(3P)-3-(4-chloro-3-methanesulfonamido-1-methyl-1H-indazol-7-yl)-7-(3- |
| | methanesulfonylphenyl)-4-oxo-3H,4H-pyrido[2,3-d]pyrimidin-2-yl]-2-(3,5-difluorophenyl)ethyl]-2- |
| | [(2S,4R)-9-(difluoromethyl)-5,5-difluoro-7,8-diazatricyclo[4.3.0.0²,⁴]nona-1(6),8-dien-7-yl]acetamide |
| Example 24 | N-[(1S)-1-[(3P)-3-(4-chloro-3-methanesulfonamido-1-methyl-1H-indazol-7-yl)-7-(2,4-difluorophenyl)- |
| | 4-oxo-3H,4H-pyrido[2,3-d]pyrimidin-2-yl]-2-(3,5-difluorophenyl)ethyl]-2-[(2S,4R)-5,5-difluoro-9- |
| | (trifluoromethyl)-7,8-diazatricyclo[4.3.0.0²,⁴]nona-1(6),8-dien-7-yl]acetamide |
| Example 25 | N-[(1S)-1-[(3P)-3-(4-chloro-3-methanesulfonamido-1-methyl-1H-indazol-7-yl)-7-(2,4-dichlorophenyl)- |
| | 4-oxo-3H,4H-pyrido[2,3-d]pyrimidin-2-yl]-2-(3,5-difluorophenyl)ethyl]-2-[(2S,4R)-9-(difluoromethyl)- |
| | 5,5-difluoro-7,8-diazatricyclo[4.3.0.0²,⁴]nona-1(6),8-dien-7-yl]acetamide |
| Example 26 | N-[(1S)-1-[(3P)-3-(4-chloro-3-methanesulfonamido-1-methyl-1H-indazol-7-yl)-7-(2,4-difluoro-3- |
| | methoxyphenyl)-4-oxo-3H,4H-pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8-dien-7-yl]actamide |
| Example 27 | N-[(1S)-1-[(3P)-3-[4-chloro-3-methanesulfonamido-1-(2,2,2-trifluoroethyl)-1H-indazol-7-yl]-7-(2- |
| | fluorophenyl)-4-oxo-3H,4H-pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8-dien-7-yl]acetamide |
| Example 28 | N-[(1S)-1-[(3P)-3-(4-chloro-3-methanesulfonamido-1-methyl-1H-indazol-7-yl)-4-oxo-7-(2,4,6- |
| | trifluorophenyl)-3H,4H-pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8-dien-7-yl]acetamide |
| Example 29 | N-[(1S)-1-[(3P)-3-(4-chloro-3-methanesulfonamido-1-methyl-1H-indazol-7-yl)-7-(3-chloro-4- |
| | fluorophenyl)-4-oxo-3H,4H-pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8-dien-7-yl]acetamide |
| Example 30 | N-[(1S)-1-[(3P)-3-(4-chloro-3-methanesulfonamido-1-methyl-1H-indazol-7-yl)-7-(3-cyano-4- |
| | fluorophenyl)-4-oxo-3H,4H-pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8-dien-7-yl]acetamide |
| Example 31 | N-[(1S)-1-[(3P)-3-(4-chloro-3-methanesulfonamido-1-methyl-1H-indazol-7-yl)-6-(2- |
| | fluorophenyl)-7-methoxy-4-oxo-3H,4H-pyrido[2,3-d]pyrimidin-2-yl]-2-(3,5-difluorophenyl)ethyl]-2- |
| | [(2S,4R)-5,5-difluoro-9-(trifluoromethyl)-7,8-diazatricyclo[4.3.0.0²,⁴]nona-1(6),8-dien-7-yl]acetamide |
| Example 32 | N-[(1S)-1-[(3P)-3-[4-chloro-1-(2,2-difluoroethyl)-3-methanesulfonamido-1H-indazol-7-yl]-7-(2- |
| | fluorophenyl)-4-oxo-3H,4H-pyrido[2,3-d]pyrimidin-2-yl]-2-(3,5-difluorophenyl)ethyl]-2-[(2S,4R)- |
| | 5,5-difluoro-9-(trifluoromethyl)-7,8-diazatricyclo[4.3.0.0²,⁴]nona-1(6),8-dien-7-yl]acetamide |
| Example 33 | N-[(1S)-1-[(3P)-3-[4-chloro-1-(2,2-difluoroethyl)-3-methanesulfonamido-1H-indazol-7-yl]-7-(2- |
| | fluorophenyl)-4-oxo-3H,4H-pyrido[2,3-d]pyrimidin-2-yl]-2-(3,5-difluorophenyl)ethyl]-2-(3- |
| | cyclopropyl-1H-pyrazol-1-yl)acetamide |
| Example 34 | N-[(1S)-1-[(3P)-3-(4-chloro-3-methanesulfonamido-1-methyl-1H-indazol-7-yl)-7-(2-fluorophenyl)-4- |
| | oxo-3H,4H-pyrido[2,3-d]pyrimidin-2-yl]-2-(3,5-difluorophenyl)ethyl]-2-(3-cyclopropyl-1H-pyrazol-1- |
| | yl)acetamide |
| Example 35 | N-[(1S)-1-[(3P)-3-[4-chloro-3-cyclopropanesulfonamido-1-(2,2-difluoroethyl)-1H-indazol-7-yl]-7-(2- |
| | fluorophenyl)-4-oxo-3H,4H-pyrido[2,3-d]pyrimidin-2-yl]-2-(3,5-difluorophenyl)ethyl]-2-(3-cylopropyl- |
| | 1H-pyrazol-1-yl)acetamide |
| Example 36 | N-[(1S)-1-[(3P)-3-[4-chloro-3-methanesulfonamido-1-(2,2,2-trifluoroethyl)-1H-indazol-7-yl]-7-(2- |
| | fluorophenyl)-4-oxo-3H,4H-pyrido[2,3-d]pyrimidin-2-yl]-2-(3,5-difluorophenyl)ethyl]-2-(3-cyclopropyl- |
| | 1H-pyrazol-1-yl)acetamide |
| Example 37 | N-[(1S)-1-[(3P)-3-(4-chloro-3-methanesulfonamido-1-methyl-1H-indazol-7-yl)-7-(2,4-difluorophenyl)- |
| | 4-oxo-3H,4H-pyrido[3,2-d]pyrimidin-2-yl]-2-(3,5-difluorophenyl)ethyl]-2-[(2S,4R)-9-(difluoromethyl)- |
| | 5,5-difluoro-7,8-diazatricyclo[4.3.0.0²,⁴]nona-1(6),8-dien-7-yl]acetamide |
| Example 38 | N-[(1S)-1-[(3P)-3-(4-chloro-3-methanesulfonamido-1-methyl-1H-indazol-7-yl)-7-(2-fluorophenyl)- |
| | 4-oxo-3H,4H-pyrido[3,2-d]pyrimidin-2-yl]-2-(3,5-difluorophenyl)ethyl]-2-[(2S,4R)-9-(difluoromethyl)- |
| | 5,5-difluoro-7,8-diazatricyclo[4.3.0.0²,⁴]nona-1(6),8-dien-7-yl]acetamide |
| Example 39 | N-[(1S)-1-[(3P)-3-(4-chloro-3-methanesulfonamido-1-methyl-1H-indazol-7-yl)-7-(2,4-difluorophenyl)- |
| | 4-oxo-3H,4H-pyrido[2,3-d]pyrimidin-2-yl]-2-(3,5-difluorophenyl)ethyl]-2-[(2S,4R)-9-(difluoromethyl)- |
| | 5,5-difluoro-7,8-diazatricyclo[4.3.0.0²,⁴]nona-1(6),8-dien-7-yl]acetamide |
| Example 40 | N-[(1S)-1-[(3P)-7-(4-chloro-2-fluorophenyl)-3-(4-chloro-3-methanesulfonamido-1-methyl-1H-indazol- |
| | 7-yl)-4-oxo-3H,4H-pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8-dien-7-yl]acetamide |

### Biological Methods

HIV cell culture assay—MT-2 cells, 293T cells and the proviral DNA clone of NL₄₋₃ virus were obtained from the NIH AIDS Research and Reference Reagent Program. MT-2 cells were propagated in RPMI 1640 media supplemented with 10% heat inactivated fetal bovine serum (FBS), 100 μg/ml penicillin G and up to 100 units/mL streptomycin. The 293T cells were propagated in DMEM media supplemented with 10% heat inactivated FBS, 100 μg/mL penicillin G and 100 μg/mL streptomycin. A recombinant NL₄₋₃ proviral clone, in which a section of the nef gene was replaced with the Renilla luciferase gene, was used to make the reference virus used in these studies. The recombinant virus was prepared through transfection of the recombinant NL₄₋₃ proviral clone into 293T cells using Transit-293 Transfection Reagent from Mirus Bio LLC (Madison, Wis.). Supernatent was harvested after 2-3 days and the amount of virus present was titered in MT-2 cells using luciferase enzyme activity as a marker by measuring luciferase enzyme activity. Luciferase was quantitated using the EnduRen Live Cell Substrate from Promega (Madison, Wis.). Antiviral activities of compounds toward the recombinant virus were quantified by measuring luciferase activity in MT-2 cells infected for 4-5 days with the recombinant virus in the presence of serial dilutions of the compound.

The 50% effective concentration (EC₅₀) was calculated by using the exponential form of the median effect equation where (Fa)=1/[1+(ED₅₀/drug conc.)m] (Johnson V A, Byington R T. Infectivity Assay. In Techniques in HIV Research. ed. Aldovini A, Walker B D. 71-76. New York: Stockton Press. 1990).

Compound cytotoxicity and the corresponding CC₅₀ values were determined using the same protocol as described in the antiviral assay except that uninfected cells were used. Cytotoxicity was assessed on day 4 in uninfected MT2 cells by using a XTT (2,3-bis[2-Methoxy-4-nitro-5-sulfophenyl]-2H-tetrazolium-5-carboxyanilide inner salt)-based colorimetric assay (Sigma-Aldrich, St Louis, Mo.).

| Example | EC₅₀ nM | CC₅₀ μM |
|---|---|---|
| Example 1 | 0.06 | >1 |
| Example 2 | 0.25 | >1 |
| Example 3 | 0.11 | >0.1 |
| Example 4 | 0.092 | >0.1 |
| Example 5 | 0.16 | >0.5 |
| Example 6 | 0.192 | >0.1 |
| Example 7 | 0.056 | >0.1 |
| Example 8 | 0.067 | >20 |
| Example 9 | 0.12 | >0.5 |
| Example 10 | 0.097 | >0.1 |
| Example 11 | 0.15 | >0.1 |
| Example 12 | 0.21 | >0.1 |
| Example 14 | 0.084 | >0.1 |
| Example 15 | 0.035 | >0.1 |
| Example 16 | 0.056 | >0.5 |
| Example 17 | 0.065 | >0.1 |
| Example 18 | 0.036 | >0.1 |
| Example 19 | 0.12 | >0.5 |
| Example 21 | 0.080 | >0.1 |
| Example 22 | 0.10 | >0.1 |
| Example 23 | 0.065 | >0.1 |
| Example 24 | 0.067 | >0.5 |
| Example 25 | 0.42 | >0.1 |
| Example 26 | 0.062 | >0.1 |
| Example 27 | 0.088 | >0.5 |
| Example 28 | 0.032 | >0.1 |
| Example 29 | 0.12 | >0.1 |
| Example 30 | 0.078 | >0.1 |
| Example 31 | 0.16 | >0.5 |
| Example 32 | 0.077 | >0.5 |
| Example 33 | 0.15 | >0.5 |
| Example 34 | 0.089 | >0.5 |
| Example 35 | 0.20 | >0.5 |
| Example 36 | 0.16 | >0.5 |
| Example 37 | 2.0 | >0.5 |
| Example 38 | 1.9 | >0.5 |
| Example 39 | 0.047 | >0.5 |

What is claimed is:

1. A compound of Formula I, or a pharmaceutically acceptable salt thereof: 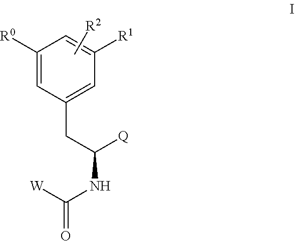
wherein:
R⁰, R¹, and R² are each independently selected from hydrogen, Cl, F, —OMe, —CN, or —CH₃ with the proviso that substituents Cl, —OMe, and —CH₃ may not be used more than twice and substituent —CN may not be used more than once;
Q is selected from: 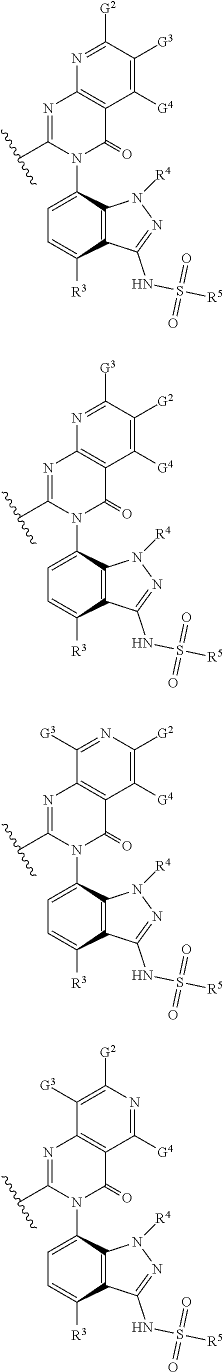 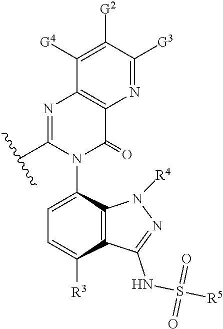
G² is one of the following: 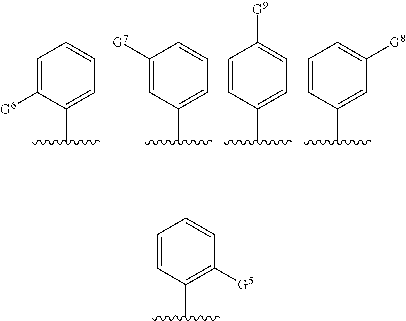
wherein the benzene ring may be further substituted up to two times with fluorine and up to two times with chlorine with the proviso that no more than three substituents directly connected to the benzene ring are a halogen and no more than two substituents directly connected to the benzene ring are a chloride;
G³ and G⁴ are independently selected from hydrogen, methyl, fluoro, chloro, or OC₁-C₂ alkyl with the proviso that at least one of G³ and G⁴ must be hydrogen;
G⁵ is hydrogen, methyl, fluoro, chloro, OC₁-C₃ alkyl, cyano, —CH₂OH, or —SO₂(C₁-C₃ alkyl);
G⁶ is hydrogen, methyl, fluoro, chloro, or OC₁-C₃ alkyl;
G⁷ is hydrogen, methyl, fluoro, chloro, OC₁-C₃ alkyl, or COOH;
G⁸ is hydrogen, C₁-C₄ alkyl, fluoro, chloro, OC₁-C₃ alkyl, COOH, —CN, —CH₂OH, or —SO₂(C₁-C₃ alkyl)
G⁹ is hydrogen, C₁-C₄ alkyl, fluoro, chloro, OC₁-C₃ alkyl, COOH, CO-morpholine, C(CH₃)₂CH₂OH, or —SO₂-morpholine wherein C₁-C₄ alkyl is optionally substituted with 1-3 fluorines;
R³ is hydrogen, Cl, or F;
R⁴ is hydrogen, C₁-C₃ alkyl, or cyclopropyl wherein C₁-C₃ alkyl is optionally substituted with 1-3 fluorines and cyclopropyl is optionally substituted with 1-2 fluorines.
R⁵ is C₁-C₃ alkyl or C₃-C₄ cycloalkyl;
W is selected from: 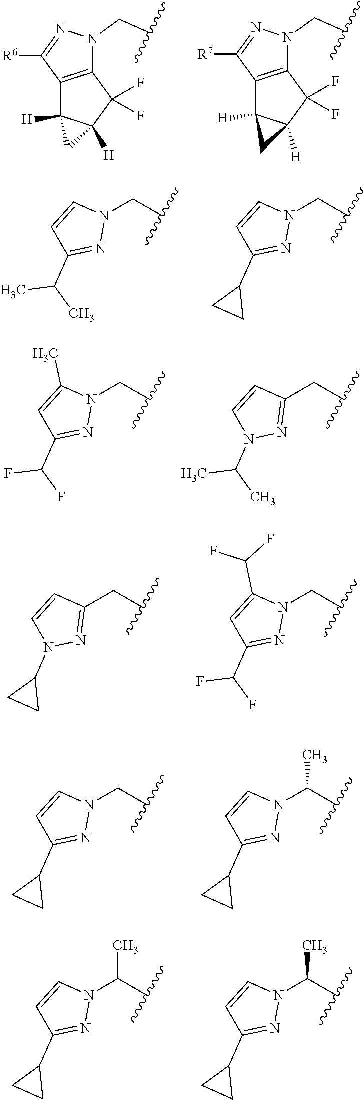
wherein R⁶ and R⁷ are independently selected from methyl optionally substituted with 1 to 3 fluorines.

2. A compound or salt according to claim 1 wherein W is selected from the following: 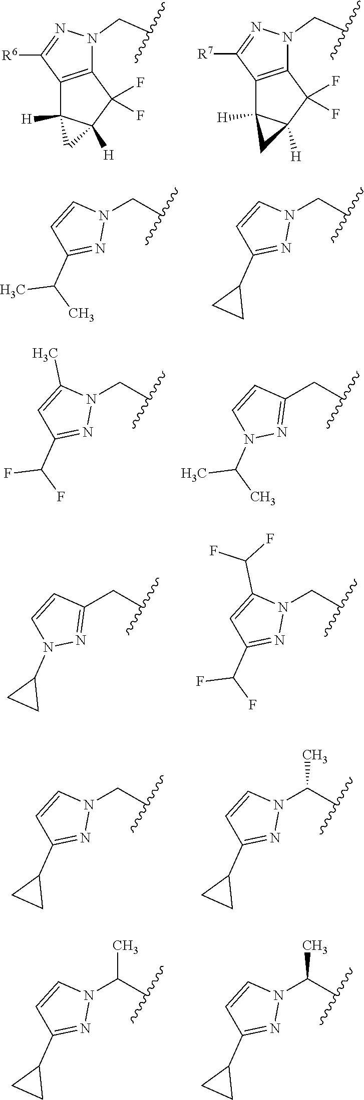
wherein R⁶ is methyl optionally substituted with one fluorine and R⁷ is methyl optionally substituted with 1 to 3 fluorines.

3. A compound or salt according to claim 1 or claim 2 wherein W is

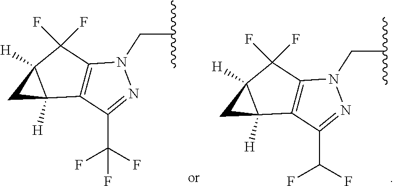 [SMILES: *Cn1nc(C(F)(F)F)c2c1C(F)(F)[C@@H]1C[C@H]21 ; *Cn1nc(C(F)F)c2c1C(F)(F)[C@@H]1C[C@H]21]

4. A compound or salt according to any of claims 1-3 wherein G² is one of the following:

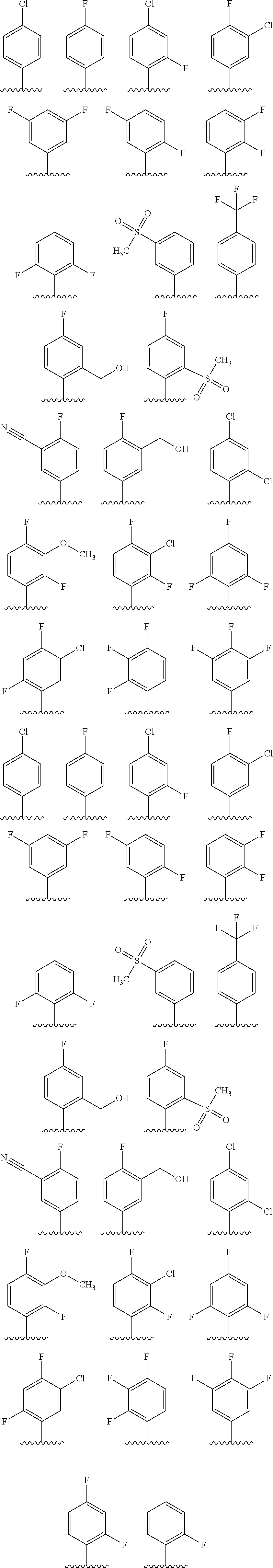

5. A compound or salt according to any of claims 1-4 wherein Q is

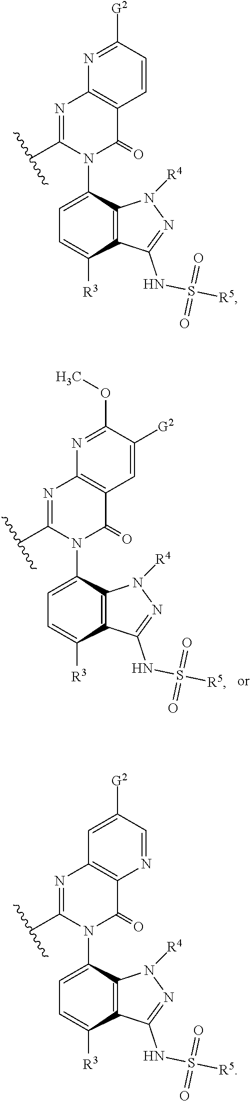

6. A compound or salt according to any of claims 1-4 wherein Q is

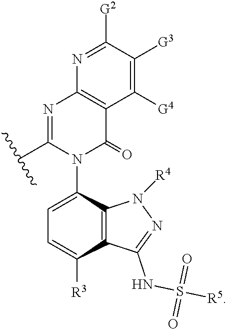

7. A compound or salt according to any of claims 1-4 wherein Q is one of the following:

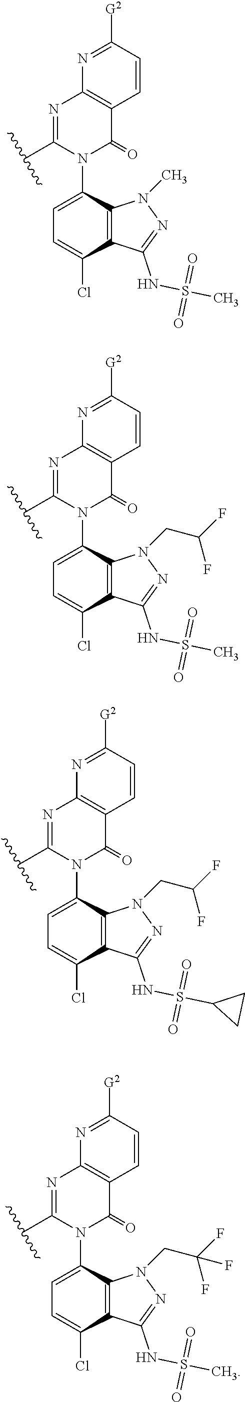

8. A compound or salt according to any of claims 1-7 wherein R⁰ is F, R¹ is F, and R² is H.

9. A compound or salt according to any of claims 1-8 wherein R⁰, R¹, and R² are each independently selected from hydrogen, F, Cl or —CH₃ with the proviso that at least one of the group R⁰, R¹ and R² is hydrogen and that R² is not hydrogen if R⁰ and R¹ are both F.

10. A compound or salt according to any of claims 1-8 wherein R⁰, R¹, and R² are each independently selected from Cl, F, —OMe, —CN, or —CH₃ with the proviso that substituents Cl, —OMe, and —CH₃ may not be used more than twice and substituent —CN may not be used more than once.

11. A compound or salt according to any of claims 1-10 wherein R³ is chloride, R⁴ is methyl, 2,2-difluoroethyl, or 2,2,2-trifluoroethyl; and R is methyl or cyclopropyl.

12. A compound or salt according to claim 1, selected from the group consisting of: 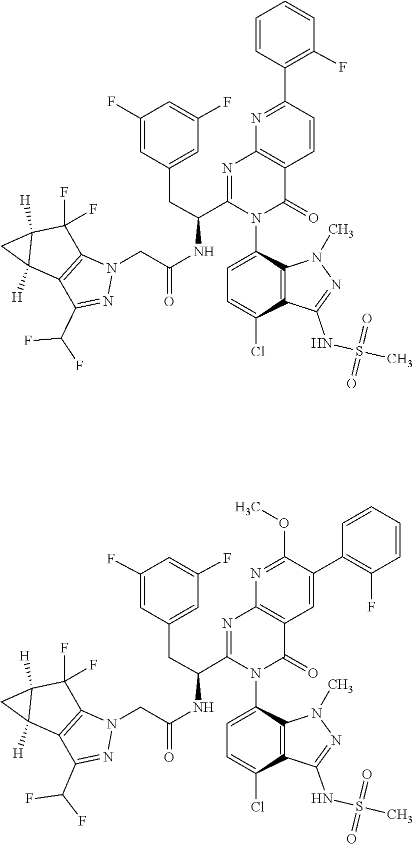 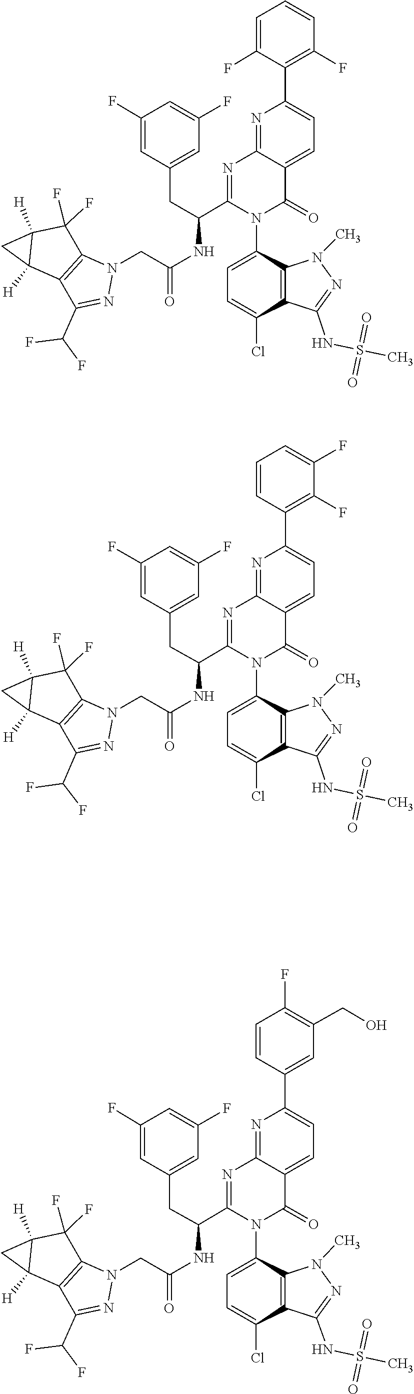 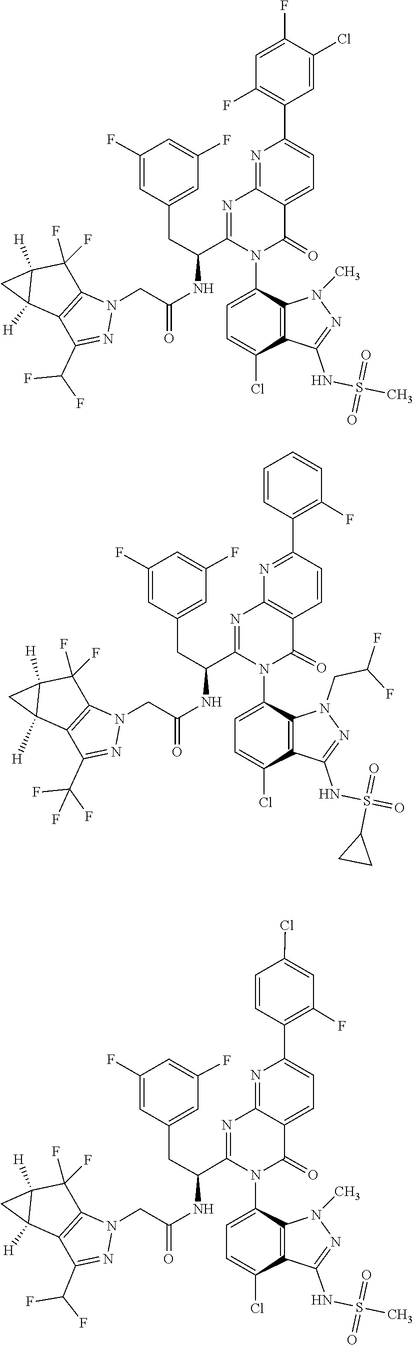 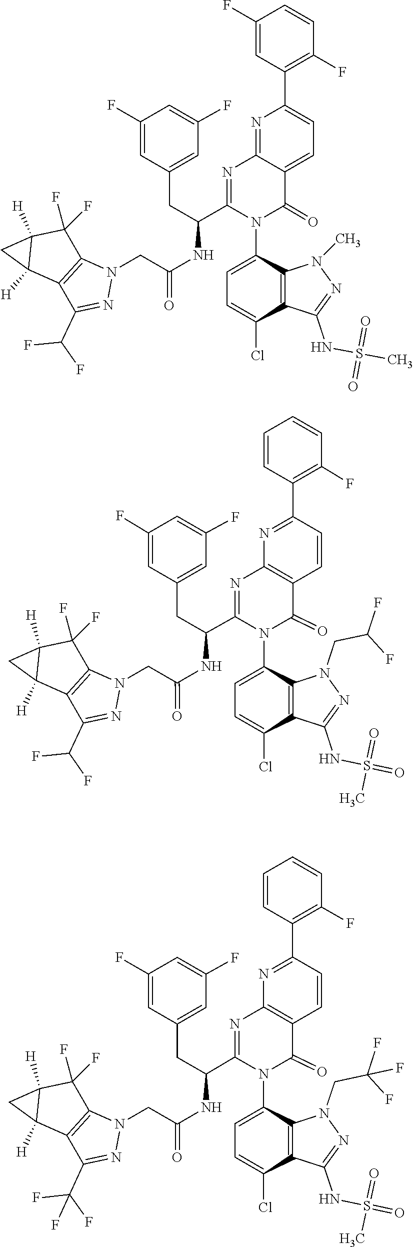 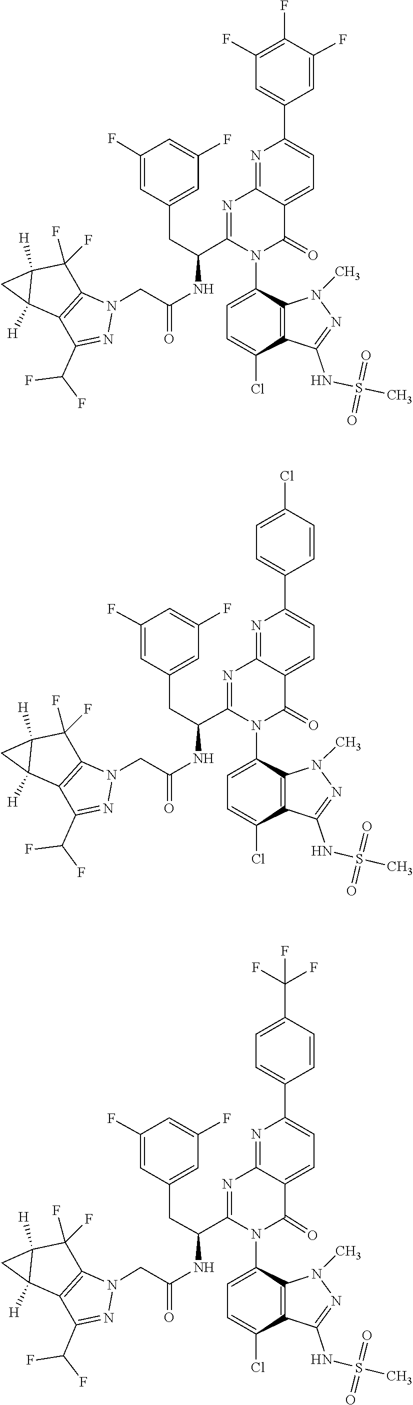 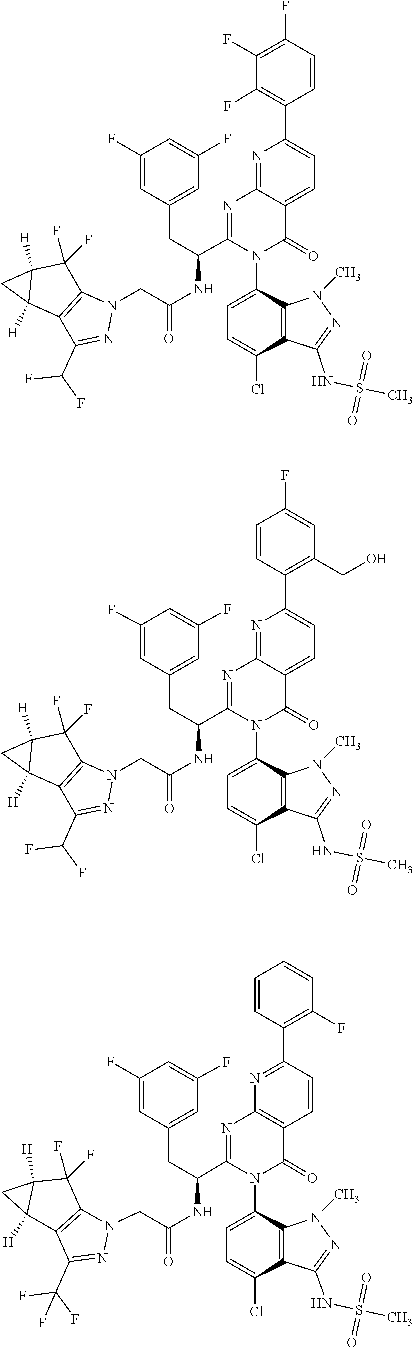 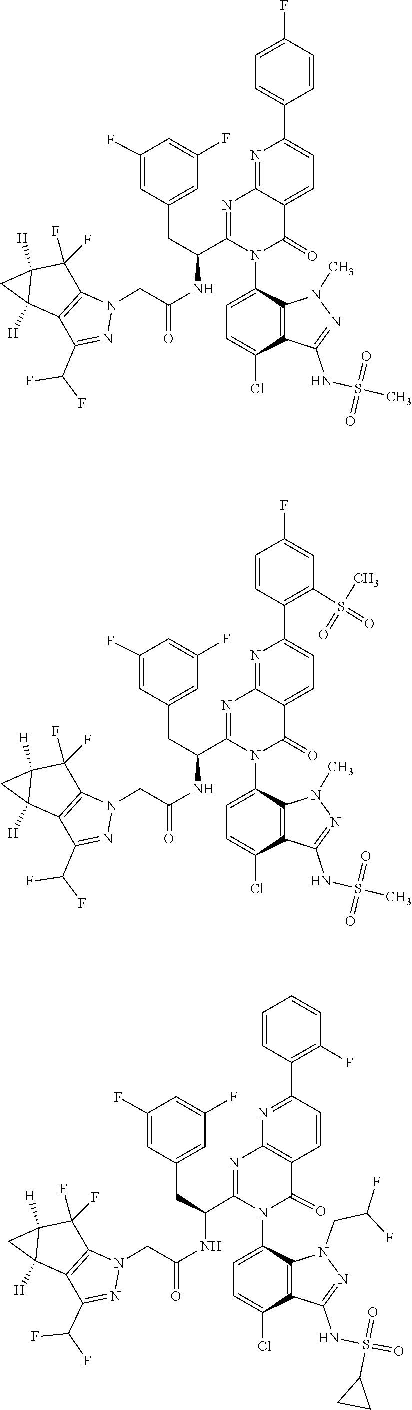 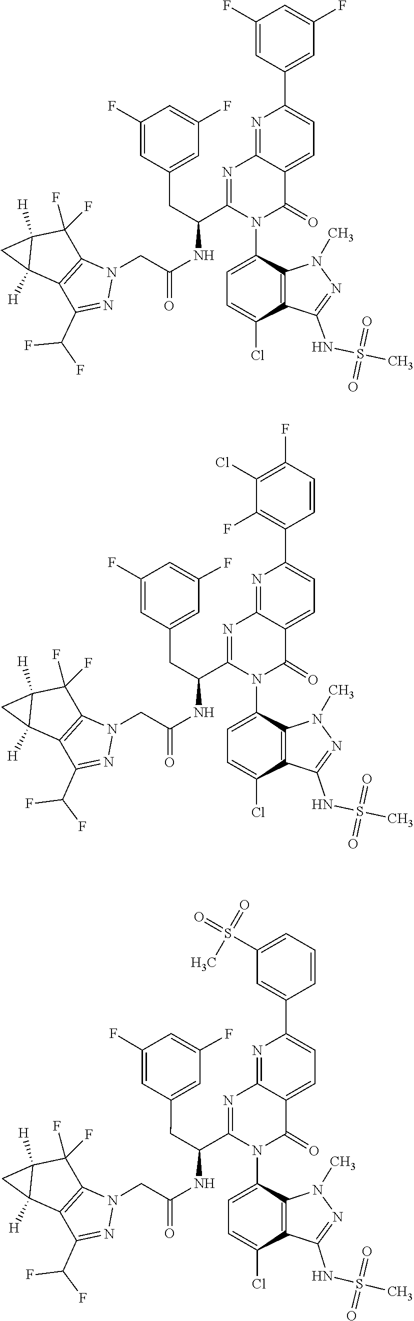 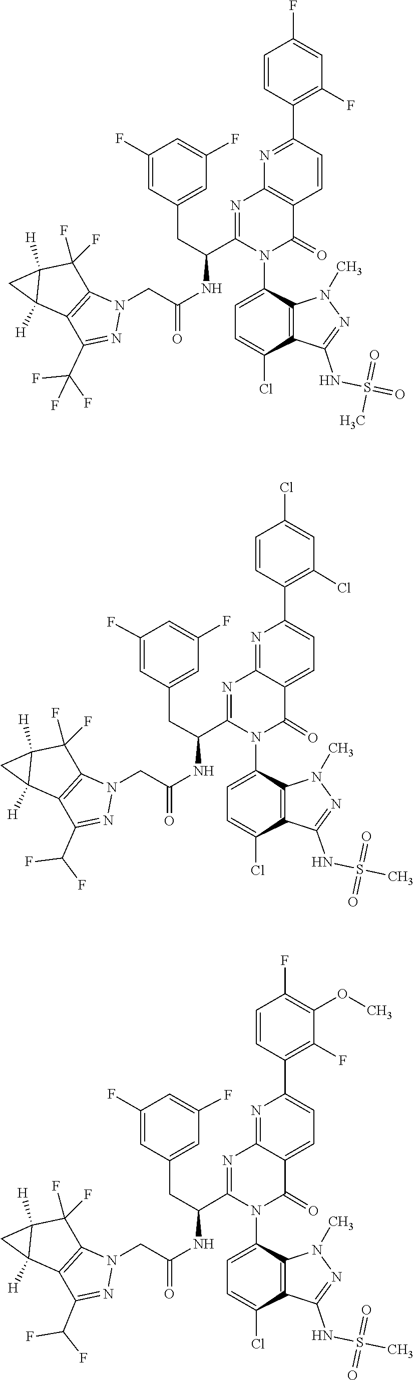 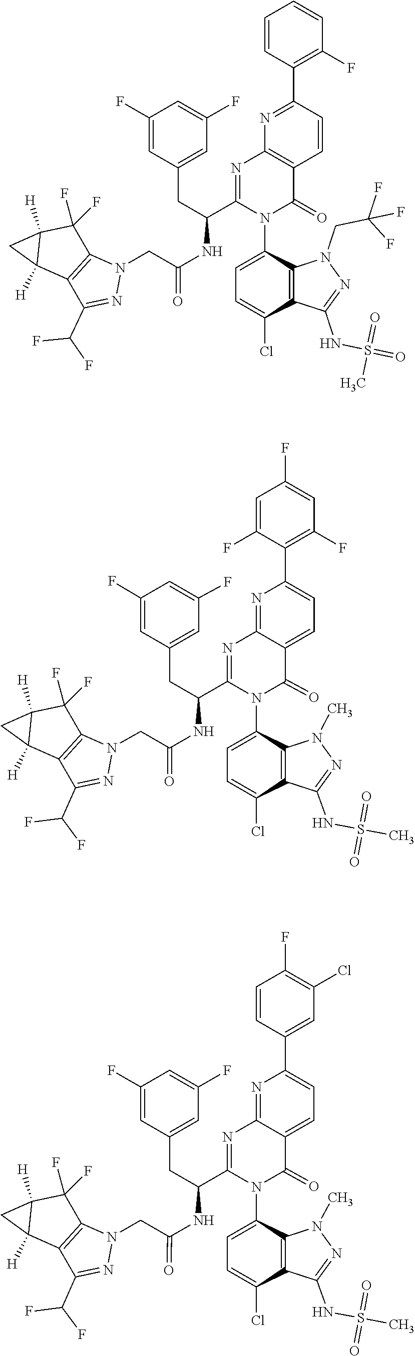 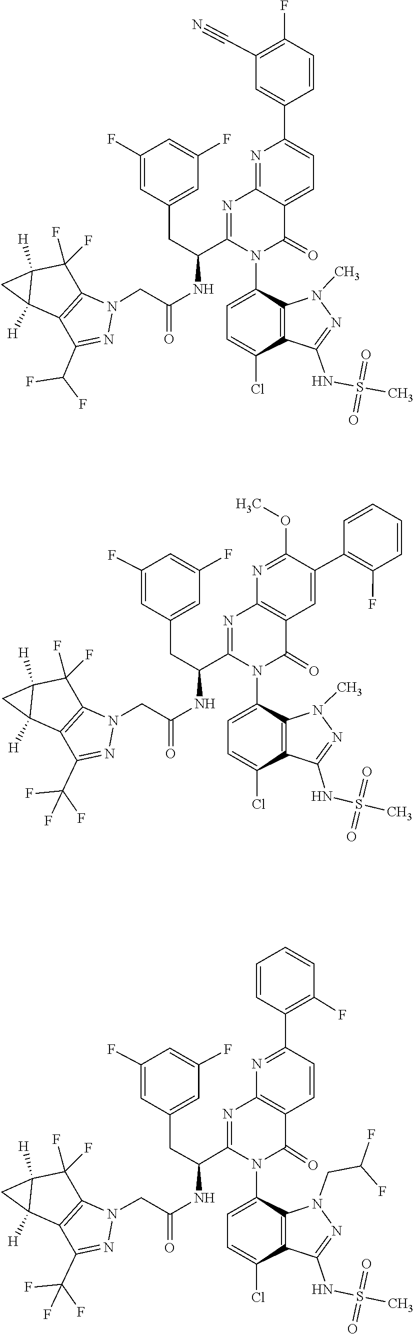 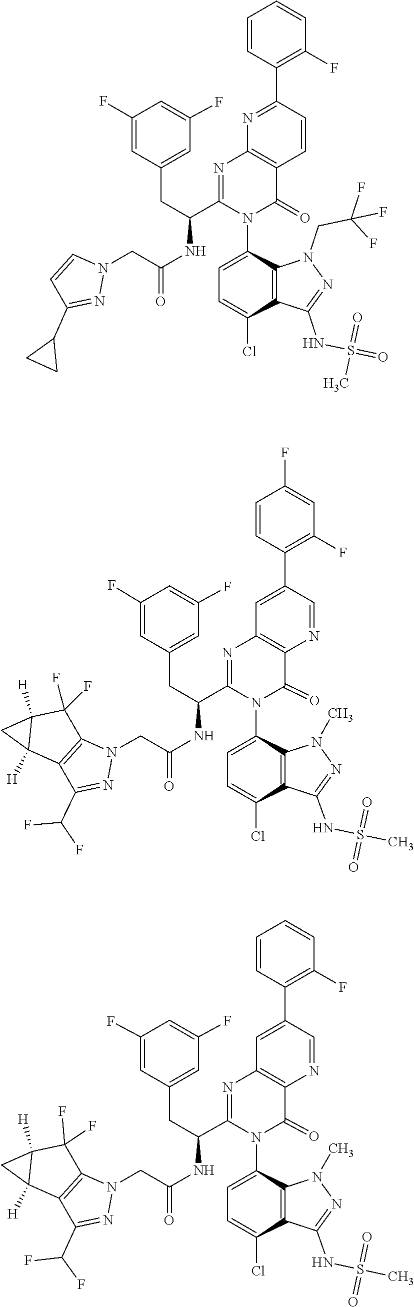 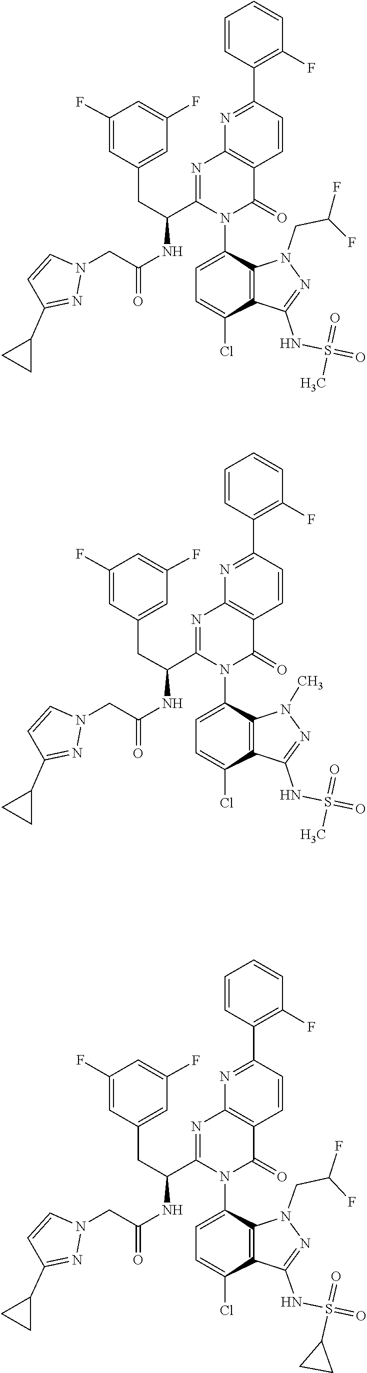 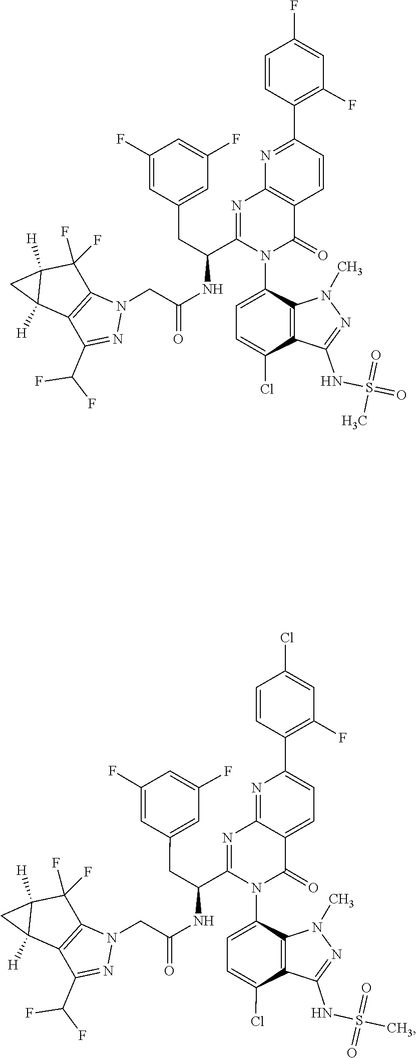
and pharmaceutically acceptable salts thereof.

13. A pharmaceutical composition comprising a compound or salt according to any of claims 1-12.

14. A composition according to claim 13 further comprising a pharmaceutically acceptable carrier, excipient, and/or diluent.

15. A method of treating HIV infection comprising administering a composition according to claim 13 or claim 14 to a patient.

16. The method of claim 15 wherein said administration is oral.

17. The method of claim 15 wherein said administration comprises administering by intramuscular injection

18. The method of claim 15 wherein said administration comprises administering by subcutaneous injection.

19. The method of claim 15 wherein said method further comprises administration of at least one other agent used for treatment of AIDS or HIV infection selected from the group consisting of nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

20. The method of claim 19 wherein said at least one other agent is selected from the group consisting of Dolutegravir, lamivudine, Fostemsavir, Cabotegravir, maraviroc, rilpiverine, Reyataz, Tenofovir, Afenamide, EfDA, Doravirine, and Preziata.

21. The method of claim 20 wherein said at least one other agent is selected from the group consisting of Dolutegravir, lamivudine, Fostemsavir, and Cabotegravir.

22. A compound or pharmaceutically acceptable salt thereof according to any of claims 1-12 for use in therapy

28. A compound or pharmaceutically acceptable salt thereof according to any of claims 1-12 for use in treating HIV infection.

29. A compound or pharmaceutically acceptable salt thereof according to any of claims 1-12 for use in the manufacture of a medicament for the treatment of HIV infection.
